SEQUENCE LISTING (66 sequences)

SEQ ID NO: 1 - protein, 7 aa, synthetic construct
CPSQCSC

SEQ ID NO: 2 - protein, 7 aa, synthetic construct
CPSRCSC

SEQ ID NO: 3 - protein, 7 aa, synthetic construct
CPAQCSC

SEQ ID NO: 4 - protein, 7 aa, unidentified
CPSQCLC

SEQ ID NO: 5 - protein, 7 aa, synthetic construct
CPSQCPC

SEQ ID NO: 6 - protein, 7 aa, synthetic construct
NGATCKK

SEQ ID NO: 7 - protein, 7 aa, synthetic construct
NEALCKK

SEQ ID NO: 8 - protein, 22 aa, synthetic construct
  VARIANT 13: note = P or L
  VARIANT 15: note = S, Y, N or H
KNWIVQHASIVNXXXGGVDNVK

SEQ ID NO: 9 - protein, 23 aa, synthetic construct
  VARIANT 13: note = P or L
  VARIANT 16: note = S, Y, N or H
KNWIVQHASIVNXXXXGGVDNVK

SEQ ID NO: 10 - protein, 19 aa, synthetic construct
TNTPVRAVTEASTSPSKCP

SEQ ID NO: 11 - protein, 11 aa, synthetic construct
SGKPVRSIICP

SEQ ID NO: 12 - protein, 18 aa, synthetic construct
SSKAVLDVTEEEAAEDCV

SEQ ID NO: 13 - protein, 18 aa, synthetic construct
QSKAVLEITEKDAASDCV

SEQ ID NO: 14 - protein, 193 aa, synthetic construct
ACPSQCSCPGTDVNCHERRLASVPAEIPTTTKILRLYINQITKLEPGVFDRLTQLTQLGLWDNQLQALPEGVFDRLVNLQKLYLNQNQLLALPVGVFDKLTQLTYLDLNNNQLKSIPRGAFDNLKSLTHIWLYGNPWDCECSDILYLKNWIVQHASIVNPHPYGGVDNVKCSGTNTPVRAVTEASTSPSKCPG

SEQ ID NO: 15 - protein, 199 aa, synthetic construct
ACPSQCSCPGTDVNCHERRLASVPAEIPTTTKILRLYINQITKLEPGVFDRLTQLTQLGLWDNQLQALPEGVFDRLVNLQKLYLNQNQLLALPVGVFDKLTQLTYLDLNNNQLKSIPRGAFDNLKSLTHIWLYGNPWDCECSDILYLKNWIVQHASIVNPHPYGGVDNVKCSGTNTPVRAVTEASTSPSKCPGYVATTT

SEQ ID NO: 16 - protein, 199 aa, synthetic construct
CPAACTCSNNIVDCRGKGLTEIPTNLPETITEIRLEQNTIKVIPPGAFSPYKKLRILRLYINQISELAPDAFQGLRSLNQLVLWDNKITELPKSLFEGLFSLQLLYLNANKINCLRVDAFQDLHNLNYLDLNNNKLQTIAKGTFSPLRAIQHIWLYGNPFICDCHLKWLADYLHASIVNPHPYGGVDNARCTSPRRLAN

SEQ ID NO: 17 - protein, 200 aa, synthetic construct
CPAACTCSNNIVDCRGKGLTEIPTNLPETITEIRLEQNTIKVIPPGAFSPYKKLRILRLYINQISELAPDAFQGLRSLNQLVLWDNKITELPKSLFEGLFSLQLLYLNANKINCLRVDAFQDLHNLNYLDLNNNKLQTIAKGTFSPLRAIQHIWLYGNPFICDCHLKWLADYLHHASIVNPHPYGGVDNARCTSPRRLAN

SEQ ID NO: 18 - protein, 200 aa, synthetic construct
CPAACTCSNNIVDCRGKGLTEIPTNLPETITEIRLEQNTIKVIPPGAFSPYKKLRILRLYINQISELAPDAFQGLRSLNQLVLWDNKITELPKSLFEGLFSLQLLYLNANKINCLRVDAFQDLHNLNYLDLNNNKLQTIAKGTFSPLRAIQHIWLYGNPFICDCHLKWLADYLHTHASIVNPHPYGGVDNRCTSPRRLAN

SEQ ID NO: 19 - protein, 202 aa, synthetic construct
CPAACTCSNNIVDCRGKGLTEIPTNLPETITEIRLEQNTIKVIPPGAFSPYKKLRILRLYINQISELAPDAFQGLRSLNQLVLWDNKITELPKSLFEGLFSLQLLYLNANKINCLRVDAFQDLHNLNYLDLNNNKLQTIAKGTFSPLRAIQHIWLYGNPFICDCHLKWLADYLHTNHASIVNPHPYGGVDNARCTSPRRLAN

SEQ ID NO: 20 - protein, 202 aa, synthetic construct
CPAACTCSNNIVDCRGKGLTEIPTNLPETITEIRLEQNTIKVIPPGAFSPYKKLRILRLYINQISELAPDAFQGLRSLNQLVLWDNKITELPKSLFEGLFSLQLLYLNANKINCLRVDAFQDLHNLNYLDLNNNKLQTIAKGTFSPLRAIQHIWLYGNPFICDCHLKWLADYLHTNPHASIVNPHPYGGVDNRCTSPRRLAN

SEQ ID NO: 21 - protein, 249 aa, synthetic construct
ACPSQCSCSGTTVNCKSKSLASVPAGIPTTTRVLYLNDNQITKLEPGVFDRLVNLQTLWLNNNQLTSLPAGLFDSLTQLTILALDSNQLQALPVGVFGRLVDLQQLYLGSNQLSALPSAVFDRLVHLKELLMCCNKLTELPRGIERLTHLTHLALDQNQLKSIPHGAFDRLSSLTHAYLFGNPWDCECRDIMYLRNWVADHTSIVMRWDGKAVNDPDSAKCAGTNTPVRAVTEASTSPSKCPGYVATTT

SEQ ID NO: 22 - protein, 244 aa, synthetic construct
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKS

SEQ ID NO: 23 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 24 - protein, 107 aa, Homo sapiens [sequence omitted]

SEQ ID NO: 25 - protein, 449 aa, Homo sapiens
QVQLVQSGGGVVQPGRSLRLSCAASGFTFSRYTIHWVRQAPGKGLEWVAVMSYNGNNKHYADSVNGRFTISRNDSKNTLYLNMNSLRPEDTAVYYCARIRDTAMFFAHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 330 aa, synthetic construct [sequence omitted]

SEQ ID NO: 27 - protein, 19 aa, synthetic construct
MEWSWVFLFFLSVTTGVHS

SEQ ID NO: 28 - protein, 4 aa, synthetic construct
GSGS

SEQ ID NO: 29 - protein, 4 aa, synthetic construct
GGGS

SEQ ID NO: 30 - protein, 10 aa, synthetic construct
GGGGSGGGGS

SEQ ID NO: 31 - protein, 20 aa, synthetic construct
GGGGSGGGGSGGGGSGGGGS

SEQ ID NO: 32 - protein, 15 aa, synthetic construct
GGGGSGGGGSGGGGS

SEQ ID NO: 33 - protein, 9 aa, synthetic construct
GGGGAGGGG

SEQ ID NO: 34 - protein, 254 aa, synthetic construct
EVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 35 - protein, 12 aa, synthetic construct
GGGSGGGSGGGS

SEQ ID NO: 36 - protein, 24 aa, synthetic construct
GGGSGGGSGGGSGGGSGGGSGGGS

SEQ ID NO: 37 - protein, 9 aa, synthetic construct
SSGSSGSSG

SEQ ID NO: 38 - protein, 11 aa, synthetic construct
SGGPSGGPSGG

SEQ ID NO: 39 - protein, 14 aa, synthetic construct
SSGSSGSSGSPVGG

SEQ ID NO: 40 - protein, 28 aa, synthetic construct
GGSGGSSPVSTPPTPSPSTPPTPSPGGS

SEQ ID NO: 41 - protein, 44 aa, synthetic construct
GGSGGSSPVSTPPTPSPSTPPTPSPSTPPTPSPSTPPTPSPGGS

SEQ ID NO: 42 - protein, 15 aa, synthetic construct
GGSGSPGSPGSPGGS

SEQ ID NO: 43 - protein, 21 aa, synthetic construct
SSGEAAAKEAAAKEAAAKSSG

SEQ ID NO: 44 - protein, 20 aa, synthetic construct
SSGEAAAKALEAEAAAKSSG

SEQ ID NO: 45 - protein, 16 aa, synthetic construct
GSSPGSSPGSSPGGGS

SEQ ID NO: 46 - protein, 33 aa, synthetic construct
GAAPAAAPAKQEAAAPAPAAKAEAPAAAPAAKA

SEQ ID NO: 47 - protein, 18 aa, synthetic construct
SSGGGPQGGPQGGPQSSG

SEQ ID NO: 48 - protein, 9 aa, synthetic construct
GGSSPGGSS

SEQ ID NO: 49 - protein, 19 aa, synthetic construct
GGSSPGGSSPGGSSPGGSS

SEQ ID NO: 50 - protein, 73 aa, synthetic construct
KLSGGGGSGGGGSGGGGSAEAWYNLGNAYYKQGDYQKAIEYYQKALELDPNNLQRSAGGGGSGGGGSGGGGAS

SEQ ID NO: 51 - protein, 453 aa, synthetic construct
MEWSWVFLFFLSVTTGVHSACPSQCSCPGTDVNCHERRLASVPAEIPTTTKILRLYINQITKLEPGVFDRLTQLTQLGLWDNQLQALPEGVFDRLVNLQKLYLNQNQLLALPVGVFDKLTQLTYLDLNNNQLKSIPRGAFDNLKSLTHIWLYGNPWDCECSDILYLKNWIVQHASIVNPHPYGGVDNVKCSGTNTPVRAVTEASTSPSKCPGYVATTTGGGGAGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 52 - protein, 503 aa, synthetic construct
MEWSWVFLFFLSVTTGVHSACPSQCSCSGTTVNCKSKSLASVPAGIPTTTRVLYLNDNQITKLEPGVFDRLVNLQTLWLNNNQLTSLPAGLFDSLTQLTILALDSNQLQALPVGVFGRLVDLQQLYLGSNQLSALPSAVFDRLVHLKELLMCCNKLTELPRGIERLTHLTHLALDQNQLKSIPHGAFDRLSSLTHAYLFGNPWDCECRDIMYLRNWVADHTSIVMRWDGKAVNDPDSAKCAGTNTPVRAVTEASTSPSKCPGYVATTTGGGGAGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 556 aa, synthetic construct
MEWSWVFLFFLSVTTGVHSACPSQCSCPGTDVNCHERRLASVPAEIPTTTKILRLYINQITKLEPGVFDRLTQLTQLGLWDNQLQALPEGVFDRLVNLQKLYLNQNQLLALPVGVFDKLTQLTYLDLNNNQLKSIPRGAFDNLKSLTHIWLYGNPWDCECSDILYLKNWIVQHASIVNPHPYGGVDNVKCSGTNTPVRAVTEASTSPSKCPGYVATTTGGGGAGGGGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 334 aa, synthetic construct
MEWSWVFLFFLSVTTGVHSACPSQCSCPGTDVNCHERRLASVPAEIPTTTKILRLYINQITKLEPGVFDRLTQLTQLGLWDNQLQALPEGVFDRLVNLQKLYLNQNQLLALPVGVFDKLTQLTYLDLNNNQLKSIPRGAFDNLKSLTHIWLYGNPWDCECSDILYLKNWIVQHASIVNPHPYGGVDNVKCSGTNTPVRAVTEASTSPSKCPGYVATTTGGGGAGGGGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 809 aa, synthetic construct
MEWSWVFLFFLSVTTGVHSACPSQCSCPGTDVNCHERRLASVPAEIPTTTKILRLYINQITKLEPGVFDRLTQLTQLGLWDNQLQALPEGVFDRLVNLQKLYLNQNQLLALPVGVFDKLTQLTYLDLNNNQLKSIPRGAFDNLKSLTHIWLYGNPWDCECSDILYLKNWIVQHASIVNPHPYGGVDNVKCSGTNTPVRAVTEASTSPSKCPGYVATTTGGGGAGGGGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGAGGGGDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKS

SEQ ID NO: 56 - protein, 556 aa, synthetic construct
MEWSWVFLFFLSVTTGVHSACPSQCSCPGTDVNCHERRLASVPAEIPTTTKILRLYINQITKLEPGVFDRLTQLTQLGLWDNQLQALPEGVFDRLVNLQKLYLNQNQLLALPVGVFDKLTQLTYLDLNNNQLKSIPRGAFDNLKSLTHIWLYGNPWDCECSDILYLKNWIVQHASIVNPHPYGGVDNVKCSGTNTPVRAVTEASTSPSKCPGYVATTTGGGGAGGGGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 57 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 58 - protein, 12 aa, synthetic construct
EPKSCDKTHTCP

SEQ ID NO: 59 - protein, 113 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 60 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 62 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 64 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 65 - protein, 819 aa, synthetic construct
MEWSWVFLFFLSVTTGVHSACPSQCSCPGTDVNCHERRLASVPAEIPTTTKILRLYINQITKLEPGVFDRLTQLTQLGLWDNQLQALPEGVFDRLVNLQKLYLNQNQLLALPVGVFDKLTQLTYLDLNNNQLKSIPRGAFDNLKSLTHIWLYGNPWDCECSDILYLKNWIVQHASIVNPHPYGGVDNVKCSGTNTPVRAVTEASTSPSKCPGYVATTTGGGGAGGGGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGGGGGAGGGGEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVL

SEQ ID NO: 66 - protein, 611 aa, synthetic construct
MEWSWVFLFFLSVTTGVHSEVQLVESGGGLVQPGGSLRLSCAASGFTFSTYAMNWVRQAPGKGLEWVGRIRSKYNNYATYYADSVKGRFTISRDDSKNTLYLQMNSLRAEDTAVYYCVRHGNFGDSYVSWFAYWGQGTLVTVSSGKPGSGKPGSGKPGSGKPGSQAVVTQEPSLTVSPGGTVTLTCGSSTGAVTTSNYANWVQQKPGKSPRGLIGGTNKRAPGVPARFSGSLLGGKAALTISGAQPEDEADYYCALWYSNHWVFGGGTKLTVLGGGGAGGGGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2025/0163144 A1
(43) Pub. Date: May 22, 2025

(54) ENGINEERED VLRB ANTIBODIES WITH IMMUNE EFFECTOR FUNCTIONS

(71) Applicant: NovAb, Inc., Tucker, GA (US)

(72) Inventor: Lovick Edward Cannon, Atlanta, GA (US)

(21) Appl. No.: 18/293,242

(22) PCT Filed: Jul. 27, 2022
(86) PCT No.: PCT/US2022/074225

§ 371 Date: Jan. 29, 2024

Related U.S. Application Data
(60) Provisional application No. 63/203,616, filed on Jul. 27, 2021.

(51) Int. Cl. C07K 16/28

(52) U.S. Cl. CPC C07K 16/28; C07K 16/2809; C07K 2317/24; C07K 2317/35; C07K 2317/53; C07K 2317/622; C07K 2317/732; C07K 2317/734; C07K 2319/00

(57) ABSTRACT

Compositions for making and using chimeric variable lymphocyte receptor B (VLRB)-immunoglobulin antibodies are provided. The antibodies are typically composed of two heavy chains and lights chains formed of heavy chain and light chain fusion proteins. Typically, heavy chain fusion proteins include a first variable lymphocyte receptor B (VLRB) antigen binding domain and a CH1 immunoglobulin domain (CHI) and optionally one or more of an immunoglobulin hinge domain (hinge), a CH2 immunoglobulin domain (CH2), a CH3 immunoglobulin domain (CH3), and CH4 immunoglobulin domain (CH4). The heavy chain fusion proteins may also include a second (VLRB) antigen binding domain, a variable region of an immunoglobulin heavy chain (VH), a mono- or multivalent single-chain variable fragment (ScFv), a polypeptide ligand (L), or a polypeptide receptor (R). Typically, light chain fusion proteins include a variable lymphocyte receptor B (VLRB) antigen binding domain and a CL immunoglobulin domain (CL).

### CROSS-REFERENCE TO RELATED APPLICATION

This application is a National Phase application under 25 U.S.C. 371 of PCT/US2022/074255, filed Jul. 27, 2022, which claims priority to U.S. Patent Provisional Application No. 63/203,616 filed Jul. 27, 2021, which are specifically incorporated by reference herein in their entireties.

### REFERENCE TO SEQUENCE LISTING

The Sequence Listing submitted as an xml file named “NOVAB_101_PCT_ST26.xml,” created on Jan. 9, 2023, and having a size of 68,802 bytes is hereby incorporated by reference pursuant to 37 C.F.R. § 1.834(c)(1).

### FIELD OF THE INVENTION

The disclosed invention is generally in the field of variable lymphocyte receptors engineered to have immune effector function.

### BACKGROUND OF THE INVENTION

Unlike Ig antibodies, VLRB antibodies do not possess intrinsic capability to recruit/activate biological effector functions needed to kill tumors and virus infected cells or to neutralize pathogens, e.g., complement activation, antibody-dependent cellular cytotoxicity (ADCC), or antibody-dependent cellular phagocytosis (ADCP), and do not interact with cellular Fc receptors, including FcRn that is responsible for Ig recycling and extended blood half-life. Initial attempts to “weaponizing” VLRB antibodies included expressing them as fusions to human IgG Fc sequences. However, improved compositions with improved biological activities of the human IgG1 Fc sequence, i.e., complement activation, ADCC, ADCP and extended blood half-life afforded by FcRn binding nonetheless remain desirable.

This it is object of the invention to provide improved VLRB-based compositions and methods of use thereof.

### SUMMARY OF THE INVENTION

Compositions for making and using chimeric variable lymphocyte receptor B (VLRB)-immunoglobulin antibodies are provided. The antibodies are typically composed of two heavy chains and lights chains formed of heavy chain and light chain fusion proteins. Thus, heavy and light chain fusion proteins are also provided.

Typically, heavy chain fusion proteins include a first variable lymphocyte receptor B (VLRB) antigen binding domain and a CH1 immunoglobulin domain (CH1) or a CL immunoglobulin domain (CL). Preferably the heavy chain proteins include one or more of an immunoglobulin hinge domain (hinge), a CH2 immunoglobulin domain (CH2), a CH3 immunoglobulin domain (CH3), and CH4 immunoglobulin domain (CH4). The heavy chain fusion proteins may also include a second (VLRB) antigen binding domain, a variable region of an immunoglobulin heavy chain (VH), a mono- or multivalent single-chain variable fragment (ScFv), a polypeptide ligand (L), or a polypeptide receptor (R). Exemplary heavy chain fusion proteins domain structures are provided in Table 1.

Typically, light chain fusion proteins include a variable lymphocyte receptor B (VLRB) antigen binding domain and a CL immunoglobulin domain (CL) or a CH1 immunoglobulin domain (CH1). Exemplary domain structures are provided in Table 2.

In some embodiments one or more of the VLRB antigen binding domain and/or one or more of the VL, VH, ScFv, VHH (i.e., nanobody), L, or R bind to a cancer or tumor antigen or an antigen expressed on by an immune cell type. The immunoglobin domains are typically independently selected from a mammalian, optionally human, IgA, IgD, IgE, IgG, and IgM, or a variant thereof with at least 70% sequence identity thereto. The IgG can be IgG1, IgG2, IgG3, and/or IgG4 and/or the IgA is IgA1 and/or IgA2. In some embodiments, all of the immunoglobin domains are from the same antibody isotype. Any of the fusion proteins can further include an active agent cargo conjugated thereto.

Nucleic acids encoding the fusion proteins and vectors including the same, e.g., for recombinant expression thereof are also provided, as are cells harboring the nucleic acids.

Chimeric antibodies formed of two heavy chain fusion proteins and two light chain fusion proteins are provided. In some embodiments, the two heavy chains are the same. In other embodiments, the two heavy chains are different. In some embodiments, the two light chains are the same. In other embodiments, the two light chains are different. Desired assembly of heterotetramers can be facilitated by, for example, incorporating knobs-into-holes and/or crossmab strategies (i.e., mutations) into the heavy and/or light chains. The chimeric antibody can be monospecific, bispecific, or multispecific. Exemplary structures are provided in Tables 3 and 4, and FIGS. 2, 3A-3D, 4, 5 and 6.

In some embodiments, one or more of the heavy chain fusion proteins that form the chimeric antibody do not include a VLRB antigen binding domain, and instead may include a variable region of an immunoglobulin heavy chain (VH), a mono- or multivalent single-chain variable fragment (ScFv), a polypeptide ligand (L), or a polypeptide receptor (R). Likewise in some embodiments one or more of the light chain fusion proteins that form the chimeric antibody do not include a VLRB antigen binding domain, and instead may include a variable region of an immunoglobulin heavy chain (VH), a mono- or multivalent single-chain variable fragment (ScFv), a polypeptide ligand (L), or a polypeptide receptor (R). However, when assembled the chimeric antibody typically include at least one, preferably two or more of the same or different VLRB antigen binding domains.

In some embodiments, the chimeric antibody can bind to a cancer or tumor antigen. Additionally or alternatively, in some embodiments, the chimeric antibody can bind to an immune cell such as a T cell, natural killer (NK) cell or a macrophage. In some embodiments, the chimeric antibody has antibody-dependent cellular cytotoxicity (ADCC), complement-dependent cytotoxicity (CDC), and/or antibody-dependent cellular phagocytosis (ADCP) activity. The chimeric antibody can have an active agent cargo conjugated thereto.

Pharmaceutical compositions including the antibodies are also provided. Typically, the compositions have an effective amount of the chimeric antibody to induce a therapeutic or diagnostic result in a subject in need thereof. The formulations are typically suitable for administration to the subject, e.g., by parenteral or enteral administration.

Methods of treating a subject in need thereof including administering the subject the chimeric antibody compositions are also provided. For example, a method of increase (e.g., induce, activate, enhance, etc.) an immune response in a subject in need thereof including administering the subject an effective amount of chimeric antibody to increase an immune response therein. A method of treating a subject for cancer can include administering the subject an effective amount of chimeric antibody to treat one or more symptoms of the cancer. In some such embodiments, the chimeric antibody binds to cells of the cancer, and optionally immune cells, and optionally, but preferably increases an immune response against the cancer cells. For example, in some embodiments, the composition recruits and/or activates immune cells against the cancer cells. A method of treating a subject for an infection can include administering the subject an effective amount of chimeric antibody to treat one or more symptoms of the infection. In some such embodiments, the chimeric antibody binds to infected cells, and optionally immune cells, and optionally, but preferably increases an immune response against the infected cells. In preferred embodiments, the immune response includes antibody-dependent cellular cytotoxicity (ADCC), complement-dependent cytotoxicity (CDC), antibody-dependent cellular phagocytosis (ADCP) activity, recruit and/or activate cytotoxic cells.

### BRIEF DESCRIPTION OF THE DRAWINGS

FIG. 1A is an illustration of the organization of a VLRB antigen binding domain. FIG. 1B is a 3-dimensional structure representation of VLRB RBC36-H3 trisaccharide antigen complex. FIG. 1C is an illustration of an IgG structure. FIG. 1D is an illustration of a bivalent VLRB-IgG Fc wherein the VLRB and Fc sequence are fused with a hinge domain.

FIG. 2 is an illustration of an engineered VLRB:CL-VLRB:CH1 tetravalent IgG chimera.

FIG. 3A is an illustration of an engineered tetravalent VLRB IgG with monovalent anti-CD3 scFv attached to the C-terminus of one of the IgG heavy (“H”) chains (“knob” H chain) using knobs-in-hole method to insure correct H chain pairing. FIG. 3B is an illustration of an engineered trivalent VLRB IgG with monovalent anti-CD3 scFv attached to the N-terminus of one of the IgG H chains (“knob” H chain) using knobs-in-hole method to insure correct H chain pairing. FIGS. 3C and 3D illustrate two ways to construct a VLRB IgG with monovalent anti-CD3 scFv attached to the C-terminus (3C) or N-terminus (3D) of one of the IgG light chains a reverse of CL and CH1 on one of the H/L chain pairs (“crossmab”) and “knobs-in-hole” to insure correct H chain pairing. FIG. 3C is an illustration of an engineered tetravalent VLRB IgG with monovalent anti-CD3 scFv attached to the C-terminus of one of the IgG L chains, which is modified by substitution of the H chain CH1 domain for the CL domain and with the complimentary H chain (“hole” H chain) modified by substitution of the CL domain for the CH1 domain. Knobs-in-hole method insures correct H chain pairing. FIG. 3D is an illustration of an engineered trivalent VLRB IgG with monovalent anti-CD3 scFv attached to the N-terminus of one of the IgG L chains, which is modified by substitution of the H chain CH1 domain for the CL domain and with the complimentary H chain (“hole” H chain) modified by substitution of the CL domain for the CH1 domain. Knobs-in-hole method insures correct H chain pairing.

FIG. 4 is an illustration of an engineered bivalent, bispecific VLRB-IgG chimera composed of an anti-CD3 monoclonal antibody with VLRB fused to the C-terminus of each CH3 domain.

FIG. 5 is an illustration of a CL-VLRB-anti-CD3 bispecific chimeric antibody.

FIG. 6 is an illustration of a VLRB:Cl-VLRB:CH tetravalent—CL:ScFv bivalent IgG structure formed of two VLRB-CH1-CH2-CH3 fusion proteins and two VLRB-CL-ScFv fusion proteins.

FIG. 7 is an illustration of the domain structure of a VLRB human IgG1 Fc fusion protein paired with a diagrammatic representation of bivalent VLRB₂ human IgG1 Fe formed therefrom.

FIG. 8 is an illustration of the domain structure of the heavy (“H”) and light chain (“L”) fusion proteins of a VLRB human IgG1 paired with a diagrammatic representation of tetravalent VLRB₄ human IgG1 formed therefrom.

FIG. 9 is an illustration of the domain structure of the heavy (“H”) and light (“L”) chain fusion proteins of a monospecific VLRB and bispecific VLRB-scFv arms paired with a diagrammatic representation of bispecific tetravalent VLRB₄-monovalent scFv₁ human IgG1 formed therefrom. In this exemplary figure: VLRB is MM3 and scFv is anti-CD3, to a form MM3 VLRB4-antiCD3 human IgG1 bispecific T cell engager (“BiTe”); “Knob” and “Hole” mutations are illustrated; and both heavy (“H”) chains have L234A/L235A/P329G mutations.

FIGS. 10A and 10B are each a series of plots showing exemplary flow cytometry data for VLRB fusion proteins and control Abs binding to: 10A) Daudi cells, CD38^{hi} and 10B) KMS-11 CD38⁻, BJAB CD38^{lo}, Raji CD38^{int}, and Daudi CD38^{hi}. D-MM3 and T-MM3 equate to MM3 VLRB₂ and VLRB₄ human IgG1 Fc fusion proteins, respectively.

FIG. 11 is a series of plots showing exemplary flow cytometry data for cell line binding of MM3 VLRB₄:anti-CD3 scFv BiTe.

FIG. 12 is a series of plots showing exemplary flow cytometry data for MM3 VLRB₄:anti-CD3 scFv BiTe binding to Jurkat and human PBMCs with anti-human CD3 antibody comparator. The Jurkat TCR/CD3 cells are from Promega's T cell activation kit catalog #1621.

FIG. 13 is a series of plots and corresponding bar graphs showing flow cytometry data for binding of O13 VLRB₂ human IgG1 Fc fusion protein (D-O13) to cell lines.

FIGS. 14A-14C show activation of Complement Dependent Cytotoxicity (CDC) and Antibody-Dependent Cellular Cytotoxicity (ADCC) by VLRB fusion proteins. FIG. 14A are flow cytometry plots showing the results of a CDC assay of Daudi cell lysis with 20% human serum and 0.5 μg of the N8 and MM3 (D-MM3) VLRB₂ human IgG1 Fe and MM3 VLRB₄ human IgG1 T-MM3) fusion proteins, and 0.25 μg daratumumab. Dead cells are detected with PI staining. FIG. 14B is a plot showing the results of a ADCC assay of Daudi cell activation of by Jurkat/FcYRIIIa V158 effector cells measured by NFAT-induced luciferase expression (Promega catalog #G7015). T-MM3 equates to MM3 VLRB₄ human IgG1 fusion protein and Dara is daratumumab. FIG. 14C is a plot showing the results of an ADCC assay of Daudi cell lysis mediated by NK92/CD16a effector cells and measured by release of LDH into the culture media. The effector cell to target cell ratio was 5:1. This assay was performed by Genescript (Piscataway, NJ). The anti-human CD20 Ab rituximab was the positive control (blue line and data points) and MM3 VLRB₂ human IgG1 Fc was the test sample (green line and data points).

FIG. 15A is a series of flow cytometry plots showing CDC activation by O13 VLRB₂ human IgG1 fusion protein. FIG. 15B is a line graph showing ADCC activation by O13 VLRB₂ human IgG1 fusion protein.

FIG. 16 is a line graph showing activation of Jurkat TCR/CD3 NFAT T cells (Promega catalog #1621). Target cell lines and activating agents are as indicated.

FIGS. 17A-17D are line graphs showing activation of human PBMC T cells by MM3 VLRB₂:anti-CD3 scFv BiTe (MM3/anti-CD3 in the Figure). Target cells are as indicated.

FIG. 18 is a bar graph showing activation of human PBMC T cell cytotoxicity. % Cytotoxicity=100×(Sample−PBMC spontaneous−Daudi spontaneous)/(Daudi maximum−Daudi spontaneous) where Sample=MM3 VLRB BiTe+PBMCs+Daudi cells, PBMC spontaneous=MM3 VLRB BiTe+PBMCs, Daudi spontaneous=PBMCs+Daudi, and Daudi maximum=Lysed Daudi.

### DETAILED DESCRIPTION OF THE INVENTION

### I. Definitions

As used herein, the term “specifically binds” refers to the binding of an antibody to its cognate antigen while not significantly binding to other antigens. Preferably, an antibody “specifically binds” to an antigen with an affinity constant (Ka) greater than about 10⁵ mol⁻¹ (e.g., 10⁶ mol⁻¹, 10⁷ mol⁻¹, 10₈ mol⁻¹, 10⁹ mol⁻¹, 10¹⁰ mol⁻¹, 10¹¹ mol⁻¹, and 10¹² mol⁻¹ or more) with that second molecule.

As used herein, the term “tumor” or “neoplasm” refers to an abnormal mass of tissue containing neoplastic cells. Neoplasms and tumors may be benign, premalignant, or malignant.

As used herein, the term “cancer” or “malignant neoplasm” refers to a cell that displays uncontrolled growth, invasion upon adjacent tissues, and often metastasis to other locations of the body.

As used herein, the term “antineoplastic” refers to a composition, such as a drug or biologic, that can inhibit or prevent cancer growth, invasion, and/or metastasis.

As used herein, the term “individual,” “host,” “subject,” and “patient” are used interchangeably to refer to any individual who is the target of administration or treatment. The subject can be a vertebrate, for example, a mammal. Thus, the subject can be a human or veterinary patient.

As used herein, the term “therapeutically effective” means that the amount of the composition used is of sufficient quantity to ameliorate one or more causes or symptoms of a disease or disorder. Such amelioration only requires a reduction or alteration, not necessarily elimination. A therapeutically effective amount of a composition for treating cancer is preferably an amount sufficient to cause tumor regression or to sensitize a tumor to radiation or chemotherapy.

As used herein, the term “pharmaceutically acceptable” refers to a material that is not biologically or otherwise undesirable, i.e., the material may be administered to a subject without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “polypeptide” refers to a chain of amino acids of any length, regardless of modification (e.g., phosphorylation or glycosylation).

As used herein, a “variant” polypeptide contains at least one amino acid sequence alteration as compared to the amino acid sequence of the corresponding wild-type polypeptide.

As used herein, an “amino acid sequence alteration” can be, for example, a substitution, a deletion, or an insertion of one or more amino acids.

As used herein, “fusion protein” refers to a polypeptide formed by the joining of two or more polypeptides through a peptide bond or other linkage formed between the amino terminus of one polypeptide and the carboxyl terminus of another polypeptide. The fusion protein can be formed by the chemical coupling of the constituent polypeptides or it can be expressed as a single polypeptide from a nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid sequence, and then expressing the nucleic acid in an appropriate host cell under conditions in which the fusion protein is produced.

As used herein, a “vector” is a replicon, such as a plasmid, phage, or cosmid, into which another DNA segment may be inserted so as to bring about the replication of the inserted segment. The vectors described herein can be expression vectors.

As used herein, an “expression vector” is a vector that includes one or more expression control sequences

As used herein, an “expression control sequence” is a DNA sequence that controls and regulates the transcription and/or translation of another DNA sequence.

As used herein, “operably linked” means incorporated into a genetic construct so that expression control sequences effectively control expression of a coding sequence of interest.

As used herein, a “fragment” of a polypeptide refers to any subset of the polypeptide that is a shorter polypeptide of the full length protein. Generally, fragments will be five or more amino acids in length.

As used herein, “valency” refers to the number of binding sites available per molecule.

As used herein, “conservative” amino acid substitutions are substitutions wherein the substituted amino acid has similar structural or chemical properties.

As used herein, “non-conservative” amino acid substitutions are those in which the charge, hydrophobicity, or bulk of the substituted amino acid is significantly altered.

As used herein, the term “host cell” refers to prokaryotic and eukaryotic cells into which a recombinant expression vector can be introduced.

As used herein, the term “identity,” as known in the art, is a relationship between two or more polypeptide sequences, as determined by comparing the sequences. In the art, “identity” also means the degree of sequence relatedness between polypeptide as determined by the match between strings of such sequences. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988).

Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). The default parameters are used to determine the identity for the polypeptides of the present disclosure.

By way of example, a polypeptide sequence may be identical to the reference sequence, that is be 100% identical, or it may include up to a certain integer number of amino acid alterations as compared to the reference sequence such that the % identity is less than 100%. Such alterations are selected from: at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the reference polypeptide by the numerical percent of the respective percent identity (divided by 100) and then subtracting that product from said total number of amino acids in the reference polypeptide.

As used herein, “optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Every compound disclosed herein is intended to be and should be considered to be specifically disclosed herein. Further, every subgroup that can be identified within this disclosure is intended to be and should be considered to be specifically disclosed herein. As a result, it is specifically contemplated that any compound, or subgroup of compounds can be either specifically included for or excluded from use or included in or excluded from a list of compounds.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular polypeptide is disclosed and discussed and a number of modifications that can be made to a number of polypeptides are discussed, specifically contemplated is each and every combination and permutation of polypeptides and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### II. VLRB-Ig Chimeras

Chimeric molecules typically composed of two heavy chains and two light chains composed of at least one, preferably two or more VLRB antigen binding domains, and optionally one or more other target moiety binding domains, each linked to immunoglobulin constant domain(s) are provided.

Referred to herein as chimeric VLRB-Ig, VLRB-Ig, chimeric antibodies, or simply antibodies, the structures are designed to mimic the natural Y-shaped structure of antibodies, increase valency of antigen binding domains, increase the number of targets that can be specifically bound, increase immune effector response(s), or a combination thereof. Elements of the design, manufacture, and methods of use are discussed in more detail below.

### A. Design Strategy

In the lamprey and hagfish, the only surviving jawless vertebrates, variable lymphocyte receptors (VLRs) play the major role in recognition of foreign antigens. In contrast to the variable, diverse, and joining gene segments (VDJs) of immunoglobulins in jawed vertebrates, the jawless vertebrates have solved the receptor diversity problem by somatic DNA rearrangement of diverse leucine-rich repeat (LRR) modules into incomplete vlr genes. The resulting mature vlr genes encode an N-terminal LRR capping region (LRRNT), the first LRR (LRR1), up to seven 24-residue variable LRRs (LRRVs), a terminal or end LRRV (LRRVe), a connecting peptide (CP), a C-terminal LRR capping region (LRRCT), and a threonine/proline-rich stalk region that connects the protein to a glycosylphosphatidylinositol (GPI) anchor and a hydrophobic tail resulting in a cell surface form of VRLB (Han, et al., Science, 321(5897):1834-7 (2008)). VLRA and VLRC are intrinsic one-pass membrane proteins and are not secreted. Secreted VLRBs lack the GPI linkage and form a pentamer of dimers structure, i.e., 10 VLRB binding domains per molecule (Herrin, et al., Proc Nat Acad Sci USA. 2008; 105(6):2040-5. Epub 2008/02/02. doi: 10.1073/pnas.0711619105. PubMed PMID: 18238899; PMCID: PMC2542867.)

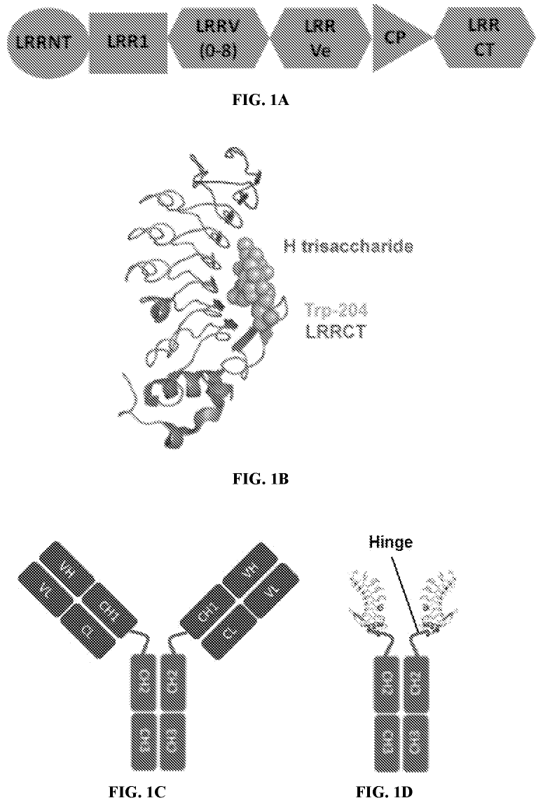

The antigen binding domain of variable lymphocyte receptors (VLRBs) is composed of a variable number of highly diverse tandemly linked leucine rich repeat (LRR) domains. These are arranged as indicated in Figure TA with an amino terminal LRR domain (LRRNT), typically 27-39 amino acids in length most typically 32 or 39 in length, a first 18 amino acid residue LRR (LRRT) followed by a variable number (none up to 8, though rarely more than 3) of LRR domains (LRRV1, LRRV2, etc.) each typically 24 or 23 amino acid residues and an end LRRV (LRRVe) also 24 amino acids in length linked via a short connecting peptide (CP) of typically 13 amino acid residues to a carboxy terminal LRR domain (LRRCT) that unlike the LRR1 and LRRV domains is of highly variable length, 48 to 63 amino acid residues (Herrin and Cooper, J Immunol. 185(3):1367-74 (2010). Epub 2010/07/28. doi: 10.4049/jimmunol.0903128. PubMed PMID: 20660361). With the exception of LRRCT all of the VLR LRR domains adopt the canonical beta strand-turn-alpha helix LRR domain 3-dimensional structure such that VLRs form a crescent “palm-like” protein structure with a variably present loop in the LRRCT domain forming a “thumb-like” cap at one end of the palm (FIG. 1B). X ray crystal structures of VLR:antigen complexes have localized antigen binding to the concave surface of VLRs (Han, et al., Science, 321(5897):1834-7 (2008). Epub 2008/09/27. doi: 10.1126/science.1162484, Collins, et al., Structure, 25(11):1667-78 e4 (2017). Epub 2017/10/11. doi: 10.1016/j.str.2017.09.003, Gunn, et al., J Mol Biol., 430(9):1350-67 (2018). Epub 2018/03/30. doi: 10.1016/j.jmb.2018.03.016., Kirchdoerfer, et al., Structure, 20:479-486 (2012). [PubMed: 22405006], Collins, et al., Acta Cryst., 682-687 (2017), Luo, et al., J Biol Chem. 288(32):23597-606 (2013). Epub 2013/06/21. doi: 10.1074/jbc.M113.480467, Velikovsky, Nature Struct. Mol. Biol. 16, 725-730 (2009)), which is a continuous β sheet surface formed by β strands of each LRR domain. Identified amino acid positions within each LRR domain are exposed to solvent on the VLR concave surface and have the potential for forming contacts with bound antigens (FIG. 1B).

However, unlike Ig antibodies, VLRB antibodies do not possess intrinsic capability to recruit/activate biological effector functions needed to kill tumors and virus infected cells or to neutralize pathogens, e.g., complement activation, antibody-dependent cellular cytotoxicity (ADCC), or antibody-dependent cellular phagocytosis (ADCP), and do not interact with cellular Fc receptors, including FcRn that is responsible for Ig recycling and extended blood half-life.

The basic structure of a naturally occurring antibody molecule is a Y-shaped tetrameric quaternary structure consisting of two identical heavy chains and two identical light chains, held together by non-covalent interactions and by inter-chain disulfide bonds. See, e.g., FIG. 1C.

In mammalian species, there are five types of heavy chains: alpha, delta, epsilon, gamma, and mu, which determine the class (isotype) of immunoglobulin: IgA, IgD, IgE, IgG, and IgM, respectively. The heavy chain N-terminal variable domain (VH) is followed by a constant region, containing three domains (numbered CH1, CH2, and CH3 from the N-terminus to the C-terminus) in heavy chains gamma, alpha and delta, while the constant region of heavy chains mu and epsilon is composed of four domains (numbered CH1, CH2, CH3 and CH4 from the N-terminus to the C-terminus). The CH1 and CH2 domains of IgA, IgG, and IgD are separated by a flexible hinge, which varies in length between the different classes and in the case of IgA and IgG, between the different subtypes: IgG1, IgG2, IgG3, and IgG4 have respectively hinges of 15, 12, 62 (or 77), and 12 amino acids, and IgA1 and IgA2 have respectively hinges of 20 and 7 amino acids.

There are two types of light chains: lamda and kappa, which can associate with any of the heavy chains isotypes, but are both of the same type in a given antibody molecule. Both light chains appear to be functionally identical. Their N-terminal variable domain (VL) is followed by a constant region consisting of a single domain termed CL.

The heavy and light chains pair by protein/protein interactions between the CH1 and CL domains, and the two heavy chains associate by protein/protein interactions between their CH3 domains. The structure of the immunoglobulin molecule is generally stabilized by interchain disulfide bonds between the CH1 and CL domains and between the hinges.

The clinical efficacy of therapeutic antibodies relies on both their antigen-binding function and their effector functions, which are respectively associated with different parts of the immunoglobulin molecule. The antigen-binding regions correspond to the arms of the Y-shaped structure, which consist each of the complete light chain paired with the VH and CH1 domains of the heavy chain, and are called the Fab fragments (for Fragment antigen binding). Fab fragments were first generated from native immunoglobulin molecules by papain digestion which cleaves the antibody molecule in the hinge region, on the amino-terminal side of the interchains disulfide bonds, thus releasing two identical antigen-binding arms. Other proteases such as pepsin, also cleave the antibody molecule in the hinge region, but on the carboxy-terminal side of the interchains disulfide bonds, releasing fragments consisting of two identical Fab fragments and remaining linked through disulfide bonds; reduction of disulfide bonds in the F(ab′)2 fragments generates Fab′ fragments.

The part of the antigen binding region corresponding to the VH and VL domains is called the Fv fragment (for Fragment variable); it contains the CDRs (complementarity determining regions), which form the antigen-binding site (also termed paratope). Besides specifically directing the antibody to its goal, the antigen-binding region upon binding to its target antigen, it may induce a variety of biological signals, which may be positive or negative depending on both the targeted antigen and the epitope recognized by the antibody on said antigen. For use in the field of cancer therapy, one generally favors antibodies delivering a growth-inhibitory or a pro-apoptotic signal, resulting in cytostasis or in death of the tumor cells (Verma et al., J Immunol, 186, 3265-76; 2011).

The effector function of the antibody results from its binding to effector molecules such as complement proteins, or to Fc receptors on the surface of immune cells such as macrophages or natural killer (NK) cells. It results in different effects leading to the phagocytosis or lysis of the targeted antigen, such as antibody dependent phagocytosis (ADP), antibody-dependent cell mediated cytotoxicity (ADCC), or complement dependent cell mediated cytotoxicity (CDC). The effector region of the antibody which is responsible of its binding to effector molecules or cells, corresponds to the stem of the Y-shaped structure, and contains the paired CH2 and CH3 domains of the heavy chain (or the CH2, CH3 and CH4 domains, depending on the class of antibody), and is called the Fc (for Fragment crystallizable) region.

The ADCC, ADP, and CDC mediated by the Fc region play a major part in the therapeutic activity of mAbs. The ADCC mechanism seems to be central, since it has been demonstrated that in nude mice genetically deficient for the Fc gamma receptor, the therapeutic action against human tumor xenografts of the two major clinically successful mAbs, anti-HER2 and anti-CD20, was almost entirely abolished (Clynes et al., Nat Med, 6, 443-6, 2000). The ADP mechanism has also been shown to be of central importance in several murine models of human tumors (Uchida et al., J. Exp. Med. 199: 1659-69, 2004), and CDC has also been demonstrated to play a fundamental role in the therapeutic activity of anti-CD20 in vivo (Di Gaetano et al., J Immunol, 171, 1581-7, 2003).

Strategies to impart these capabilities to VLRB antibodies are provided. The strategies typically include fusing or linking an antigen binding fragment thereof, and optionally one or more other cell targeting or binding moieties to molecule having a CH1 domain, CL domain or a combination thereof to form a VLRB-Ig chimera. In preferred embodiments, the chimera also includes other Ig constant domains such as CH2, CH3, hinge region, or a combination thereof, and optionally may further include one or more variable regions of an Ig. In preferred embodiments, the molecule assumes a dimeric Y-shaped structure similar to an antibody.

The structure ofthe chimeric VLRB-g, fusion proteins that can be used to build the chimera, and exemplary VLRB and Ig sequences that can be used therein are discussed in more detail below.

### B. VLRB-Ig Chimeric Fusions Proteins

The disclosed VLRB-g chimeras can be assembled using fusions proteins combining elements ofVLRB antibodies and Ig antibodies.

Heavy chain fusion protein designs include, but are not limited to, from N terminus to C terminus, the constructs outlined in Table 1:

TABLE 1
Exemplary Heavy Chain Fusion Protein Domain Structures

VLRB - CH1
VLRB - CH1 - hinge
VLRB - CH1 - hinge - CH2
VLRB - CH1 - hinge - CH2 - CH3
VLRB - CH1 - hinge - CH2 -CH3 - CH4
CH1 - hinge - CH2 - CH3 - VLRB
CH1 - hinge - CH2 - CH3 - CH4 - VLRB
VH - CH1 - hinge - CH2 - CH3 - VLRB
VH - CH1 - hinge - CH2 - CH3 - CH4 - VLRB
VLRB - CH1 - hinge - CH2 - CH3 - VLRB
VLRB - CH1 - hinge - CH2 - CH3 - CH4 - VLRB
VLRB - CH1 - hinge - CH2 - CH3 - ScFv/VHH/R/L
VLRB - CH1 - hinge - CH2 - CH3 - CH4 - ScFv/VHH/R/L
ScFv/VHH/R/L - CH1 - hinge - CH2 - CH3 - VLRB
ScFv/VHH/R/L - CH1 - hinge - CH2 - CH3 - CH4 - VLRB
ScFv/VHH/R/L - CH1
ScFv/VHH/R/L - CH1 - hinge
ScFv/VHH/R/L - CH1 - hinge - CH2
ScFv/VHH/R/L - CH1 - hinge - CH2 - CH3
ScFv/VHH/R/L - CH1 - hinge - CH2 -CH3 - CH4
ScFv/VHH/R/L - CH1 - hinge - CH2 - CH3 - ScFv/VHH/R/L
ScFv/VHH/R/L - CH1 - hinge - CH2 - CH3 - CH4-ScFv/VHH/R/L
CH1 - hinge - CH2 - CH3
CH1 - hinge - CH2 - CH3 - CH4
CH1 - hinge - CH2 - CH3 - ScFv/VHH/R/L
CH1 - hinge - CH2 - CH3 - CH4 - ScFv/VHH/R/L
VH - CH1 - hinge - CH2 - CH3
VH - CH1 - hinge - CH2 - CH3 - CH4
VH - CH1 - hinge - CH2 - CH3 - ScFv/VHH/R/L
VH - CH1 - hinge - CH2 - CH3 - CH4 - ScFv/VHH/R/L
CH2 - CH3
CH2 - CH3 - CH4
CH2 - CH3 - ScFv/VHH/R/L/VLRB
CH2 - CH3 - CH4 - ScFv/VHH/R/L/VLRB VLRB - CL - hinge
VLRB - CL - hinge - CH2
VLRB - CL - hinge - CH2 - CH3
VLRB - CL - hinge - CH2 -CH3 - CH4
CL - hinge - CH2 - CH3 - VLRB
CL - hinge - CH2 - CH3 - CH4 - VLRB
VH - CL - hinge - CH2 - CH3 - VLRB
VH - CL - hinge - CH2 - CH3 - CH4 - VLRB
VLRB - CL - hinge - CH2 - CH3 - VLRB
VLRB - CL - hinge - CH2 - CH3 - CH4 - VLRB
VLRB - CL - hinge - CH2 - CH3 - ScFv/VHH/R/L
VLRB - CL - hinge - CH2 - CH3 - CH4 - ScFv/VHH/R/L
ScFv/VHH/R/L - CL - hinge - CH2 - CH3 - VLRB
ScFv/VHH/R/L - CL - hinge - CH2 - CH3 - CH4 - VLRB
ScFv/VHH/R/L - CL
ScFv/VHH/R/L - CL - hinge
ScFv/VHH/R/L - CL - hinge - CH2
ScFv/VHH/R/L - CL - hinge - CH2 - CH3
ScFv/VHH/R/L - CL - hinge - CH2 -CH3 - CH4
ScFv/VHH/R/L - CL - hinge - CH2 - CH3 - ScFv/VHH/R/L
ScFv/VHH/R/L - CL - hinge - CH2 - CH3 - CH4 - ScFv/VHH/R/L
CL - hinge - CH2 - CH3
CL - hinge - CH2 - CH3 - CH4
CL - hinge - CH2 - CH3 - ScFv/VHH/R/L
CL - hinge - CH2 - CH3 - CH4 - ScFv/VHH/R/L
VH - CL - hinge - CH2 - CH3
VH - CL - hinge - CH2 - CH3 - CH4
VH - CL - hinge - CH2 - CH3 - ScFv/VHH/R/L
VH - CL - hinge - CH2 - CH3 - CH4 - ScFv/VHH/R/L

Light chain fusion protein designs include, but are not limit to, from N terminus to C terminus, the constructs outlined in Table 2:

TABLE 2
Exemplary Light Chain Fusion Protein Domain Structures

VLRB - CL
VL - CL
ScFv/VHH/R/L - CL
CL - VLRB
CL - ScFv/VHH/R/L
VL - CL - VLRB
VL - CL - ScFv/VHH/R/L
VLRB - CL- VLRB
VLRB - CL - ScFv/VHH/R/L
ScFv/VHH/R/L - CL - VLRB
ScFv/VHH/R/L - CL - ScFv/VHH/R/L
VLRB - CH1
VL - CH1
ScFv/VHH/R/L - CH1
CH1 - VLRB
CH1- ScFv/VHH/R/L
VL - CH1 - VLRB
VL - CH1 - ScFv/VHH/R/L
VLRB - CH1- VLRB
VLRB - CH1 - ScFv/VHH/R/L
ScFv/VHH/R/L - CH1 - VLRB
ScFv/VHH/R/L - CH1 - ScFv/VHH/R/L

In the disclosed fusion proteins, “VLRB” refers to a domain that includes a segment, fragment, or variant of a VLRB antibody that can bind an antigen. Thus, typically the VLRB domain includes at least the antigen binding domain of a VLRB (see, e.g., Figure TA and descriptions herein and else wherein in the art), or an antigen binding variant thereof having, e.g., at least 70%, 75%, 80%, 85%, 90%, 95%, or more sequence identity to the parent VLRB antigen binding domain. In some embodiments, the VLRB domain of the fusion protein is an entire VLRB antibody, or an antigen binding variant thereof having, e.g., at least 70%, 75%, 80%, 85%, 90%, 95%, or more sequence identity to the parent VLRB antibody.

In the disclosed fusion proteins, “CH1,” “CH2,” “CH3,” and “CH4” refers to the CH1, CH2, CH3, and CH4 domains of mammalian Ig, and variants thereof. In some embodiments, the domain(s) and/or variants thereof maintain one or more immunological functions of the constant region of mammalian Ig. Preferred retained functions include, but are not limited to, ADCC, ADP, and/or CDC. The Ig can be any IgA, IgD, IgE, IgG, and IgM, respectively, including all subclasses thereof.

In the disclosed fusion proteins, “hinge” refers to an Ig hinge region or domain, or another flexible linker such as those discussed below. The sequence/length of the hinge differs between the different classes and subclass of Ig. For example, IgG1, IgG2, IgG3, and IgG4 have respectively hinges of 15, 12, 62 (or 77), and 12 amino acids, and IgA1 and IgA2 have respectively hinges of 20 and 7 amino acids. Each of the constant domains can be independently selected from all of the available Ig constant domains of the suitable type (e.g., CH1, CH2, CH3, CH4, hinge, etc.). Thus, isotype mixing of the constant domains is contemplated. However, in preferred embodiments, all of the constant domains come from the same isotype.

In the disclosed fusion proteins, “VH” and “VL” refer to the variable regions of the heavy and light chain, respectively, of an antibody, or an antigen binding fragments thereof, that typically have at least 70%, 75%, 80%, 85%, 90%, 95%, or more sequence identity to the parent VH or VL domain. Collectively referred to as an Fv fragment (for Fragment variable), these domains contain three CDRs (complementarity determining regions) each, which form the antigen-binding site of Ig. Preferably the “VH” and/or “VL” domains of the fusions proteins contain all three of its parent VH and/or VL CDR sequences, or variant sequence(s) thereof with at least 70%, 75%, 80%, 85%, 90%, 95%, or more sequence identity thereto.

In the disclosed fusion proteins, “ScFv” refers to a fusion protein including variable regions of heavy (“VH”) and light (“VL”) chains, or antibody binding fragments thereof as defined in the preceding paragraph, which are joined together by a flexible peptide linker. The scFv can be in the VH-linker-VL or VL-linker-VH orientation, but is assembled in a manner suitable for the scFv to form the desired structure for antigen binding (i.e., for the V_{H} and V_{L} of the single polypeptide chain to associate with one another to form a Fv). The V_{L} and V_{H} regions may be derived from the parent antibody or may be chemically or recombinantly synthesized. “ScFv” as used herein refers not only to monovalent single-chain variable fragments (i.e., mono-scFv), but also multivalent single-chain variable fragments such as di-scFv, tri-scFv, etc., and each is specifically disclosed alone and in combination. The di-scFv and tri-scFv can be monospecific, bispecific, trispecific, etc., and each is specifically disclosed alone and in combination.

In the disclosed fusion proteins, “VHH” refers to a nanobody. Nanobodies are tiny, recombinantly produced antigen binding VHH fragments, derived from the Alpaca heavy chain IgG antibody (HCAb).

In the disclosed fusion proteins, “R” and “L” refers to a non-antibody targeting moiety or domains such as a receptor (“R”) or ligand (“L”). Thus, in some embodiments, the fusion proteins include a polypeptide receptor or polypeptide ligand in place of the antigen binding domain of an antibody. Like an antigen binding domain, the R/L domain can target and/or link the chimeric antibody to a cell surface ligand or receptor that is, for example, specifically expressed on an immune cell, for example CD8+ cytotoxic T cells, on tumor cells or tumor-associated neovasculature or are overexpressed on tumor cells or tumor-associated neovasculature as compared to normal tissue.

In the disclosed fusion proteins, “-” refers to the linkage between domains of the chimeric fusion proteins, and can also represent an optional linker domain. Typically, the domains are linked by a peptide bond(s) either directly between the adjacent domains or through an intervening flexible linker, however, chemical linkages for some or all of the linkages are also contemplated.

In the disclosed fusion proteins, “/” indicates alternatives, as also discussed in more detail below with respect to Table 3. For example, ScFv/VHWR/L indicates the domain can be an “ScFv” domain or an “R” domain or an “L” domain as defined herein.

Each of the foregoing domains, and examples thereof, are discussed in more detail below.

### C. Chimeric VLRB-Ig Structures

The disclosed fusion proteins are typically used to assemble dimers or tetramers that resemble the overall structure of a partial or full antibody. The disclosed chimeric antibodies contain one or more VLRB antigen binding domains, and many so include one or more antibody variable domains, ScFv, VHH, R, L, etc.

In some embodiments, the chimeric antibodies include one, preferably two, heavy chains, in the absence of light chains. Such antibodies typically include a hinge-CH2-CH3/CH4 structure, either as a monomer, or preferable as a hetero- or more preferably a homo dimer. Such structure are also referred to herein as IgG Fc (see, e.g., FIG. 1D). Preferably, the chimeric antibodies include at least one, preferably two, heavy chains, and one, preferably two, light chains, but may also form higher order structures.

In preferred embodiments, the chimeric antibodies form a traditional antibody structure composed of two heavy chains and two light chains. As in naturally occurring antibodies, disulfide bonds formed between the two heavy chains, and between pairs of heavy and light chains can link the chains into a tetrameter.

Typically the N-terminal end of the heavy and light chain(s) present one or more antigen binding domains (e.g., VLRB antigen binding domains, antibody variable domains, ScFv, VHH, R, L etc.). In some embodiments, the C-terminus of the heavy and/or light chain(s) alternatively or additional present antigen binding domains (e.g., VLRB antigen binding domains, ScFv, VHH, R, L etc.).

Various homodimeric and heterodimeric constructions are envisioned. For example, in some embodiments, the resulting tetramer has two identical halves, which together form the Y-like shape. Each end of the fork contains an identical antigen binding site.

In other embodiments, the halves are not identical. Thus, various combinations of identical and non-identical heavy and lights chains are provided. VLRB antigen binding domains can be combined with variable domains and/or ScFv and/or VHH and/or R and/or L. Furthermore, when two or more of the class of antigen binding domain or other targeting moiety (e.g., VLRB, antibody variable domains, ScFv, VHH, R, and/or L) are present, and each domain can be the same or different. In this way, the chimeric antibodies can be multivalent for the same antigen and/or multivalent for different antigens or other targets. Thus, the chimeric antibodies can be monospecific, bispecific, or multispecific with tunable valency.

Heterodimeration technology can be incorporated to drive assembly of the desired components and the desired locations. For example, in some embodiments, the heavy chains and optionally light chain(s) feature “knobs-into-holes” technology whereby complementary mutations are made in one or more of the domains of the heavy chains (e.g., CH3 chain domains) and/or light chain/heavy chain pairs (e.g., CL and CH1 domains) (see, e.g., Merchant, et al. “An efficient route to human bispecific IgG.” Nat Biotechnol. 1998; 16:677-81. doi: 10.1038/nbt0798-677). These noncovalent interactions, along with disulfide bridges in the hinge region, drive assembly toward heterodimer formation of antibodies having the same or different light chains (Spiess, et al., “Bispecific antibodies with natural architecture produced by co-culture of bacteria expressing two distinct half-antibodies.” Nat Biotechnol. 2013; 31:753-8. doi: 10.1038/nbt.2621.). See also, Shatz, “Knobs-into-holes antibody production in mammalian cell lines reveals that asymmetric afucosylation is sufficient for full antibody-dependent cellular cytotoxicity,” MAbs. 2013 Nov. 1; 5(6): 872-881. doi: 10.4161/mabs.26307; Rouet, et al., “Stability engineering of the human antibody repertoire”, FEBS Lett. 2014 Jan. 21; 588(2):269-77. doi: 10.1016/j.febslet.2013.11.029. Epub 2013 Nov. 28; and Rouet and Christ, “Bispecific antibodies with native chain structure.” Nat Biotechnol 32, 136-137 (2014). doi.org/10.1038/nbt.2812, each of which is specifically incorporated by reference herein in its entirety.

In some embodiment, the antibody is a “crossmab”. Based on the knobs-into-holes technology that facilitates heterodimerization of the heavy chains, correct association of the light chains and their cognate heavy chains is achieved by exchange of heavy-chain and light-chain domains within the antigen binding fragment (Fab) of one half of the bispecific (or multispecific) antibody. This “crossover” retains the antigen-binding domains but makes the two arms so different that light-chain mispairing can no longer occur. See, e.g., Schaefer, et al., “Immunoglobulin domain crossover as a generic approach for the production of bispecific IgG antibodies”, PNAS, 108(27):11187-11192 (2011), pnas.org/cgi/doi/10.1073/pnas.1019002108, which is specifically incorporated by reference herein in its entirety, particularly the CH1-CL crossmab.

For example, in some embodiments, the antibody is formed of two heavy chains of Table 1 and two light chains of Table 2, wherein the antibody includes at least 1 VRLB domain. Table 3 below provides a non-limiting, exemplary antibody genus structures, with eight locations for targeting moiety domains.

TABLE 3
Chimeric Antibody Genus Structures

3a
i - CL - v
ii - CH1 - hinge - CH2 - CH3/CH3-CH4 - vi
iii - CH1 - hinge - CH2 - CH3/CH3-CH4 - vii
iv - CL - viii
3b
i - CL1 - v
ii - CL - hinge - CH2 - CH3/CH3-CH4 - vi
iii - CH1 - hinge - CH2 - CH3/CH3-CH4 - vii
iv - CL - viii
3c
i - CL - v
ii - CH1 - hinge - CH2 - CH3/CH3-CH4 - vi
iii - CL - hinge - CH2 - CH3/CH3-CH4 - vii
iv - CH1 - viii

More specifically, the structures 3a, 3b, and 3c illustrate the N-terminus and C-terminus of the CL domains, the N-terminus of the CH1 domains, and the C-terminus CH3 or CH4 domains as fusion points for binding/targeting moieties indicated by roman numerals i, ii, iii, iv, v, vi, vii, and viii. Binding/targeting moieties (i.e., each roman numeral) can be, VLRB, VH, VL, ScFv, VHH, R, or L as discussed above. Each of these domains can be unoccupied or occupied by any of the foregoing targeting moiety domains, in every possible combination, provided at least one VLRB is present.

All subgenera and each such specific structure encompassed by the structures of Table 3 is specifically disclosed. When two or more of the same domain is present it may target the same or different antigen or binding partner. As an illustration, a structure wherein all of roman numerals i, ii, iii, iv, v, vi, vii, and viii are occupied by VLRB, each VLRB can be the same or different from others such at as few as one VLRB is present eight times, or as many as eight VLRB are present one time each, or any sub combination thereof, at any desired location. Arabic numerals can be used to represent different VLRB. For example, wherein eight different VLRB are present, they can be represented as VLRB1, VLRB2, VLRB3, VLRB4, VLRB5, VLRB6, VLRB7, or VLRB8. Similarly, where the same VLRB is used eight times, all eight VLRB can be represented as VLRB1.

Higher order combinations with VLRB, VH, VL, ScFv, VHH, R, and/or L, each directed to the same or different target are also treated in the same way and can be similarly labeled in subgenus and species structures derived from the genus structure with the same or different VH, VL, ScFv, VHH, R, and/or L at any desired position, also using Arabic numbers to illustrate different binding targets, provided at least one VLRB is present. Positions may also be left unoccupied. Typically, where a VL is present, it is fused to the N-terminus of a CL and adjacent to a paired VH fused to the N-terminus of a CH1.

In another annotated position, “CH3/CH3-CH4” means that either CH3 or CH3-CH4 can be independently selected at the referenced position.

Other terms, including but not limited to, VLRB, VL, VH, ScFv, VHH, R, L, CHL, CH1, hinge, CH2, CH3, and CH4 are defined as discussed above with respect to the fusion proteins.

Structures 3b and 3c provide exemplary “crossmab” design to drive specific desired dimerization of the light chain and heavy chain pairs.

Table 4 provides non-limiting illustrative species of the genus of Table 3, which are also illustrated in the Figures as indicated.

TABLE 4
Illustrative Chimeric Antibody Subgenus and Species Structures

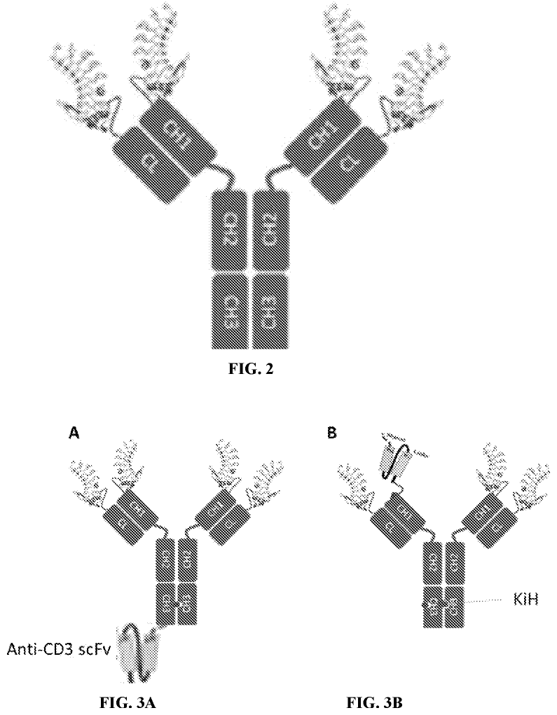

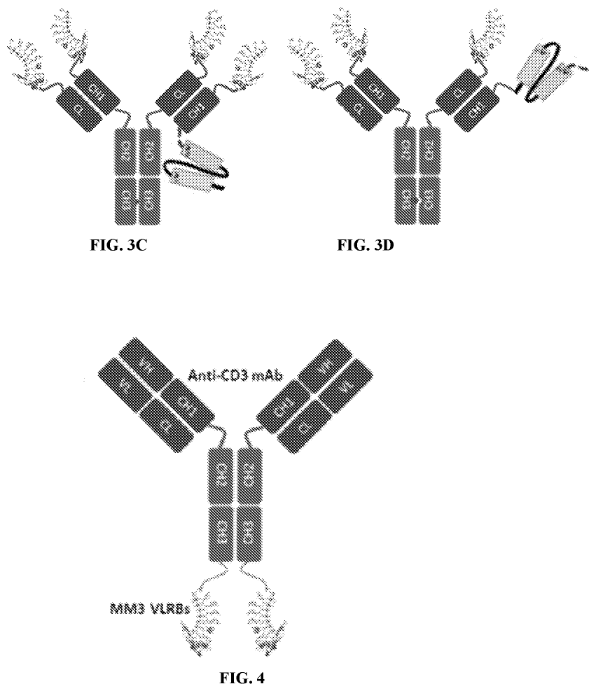

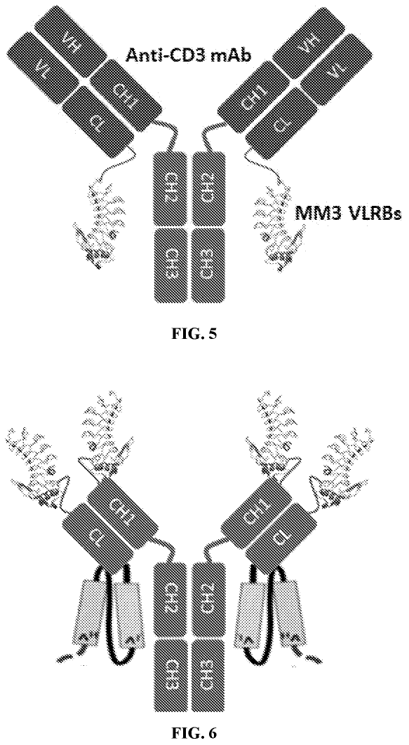

FIG. 2 can be represented as
VLRB1/2/3/4 - CL
VLRB1/2/3/4 - CH1 - hinge - CH2 - CH3
VLRB1/2/3/4 - CH1 - hinge - CH2 - CH3
VLRB1/2/3/4 - CL
which is a structure of Table 3, structure 3a, wherein each of
i-iv are occupied by 4 VLRB domains, which may the same or
different. v-viii are unoccupied. The CH3/CH3-CH4 domain is a
CH3.
FIG. 3A can be represented as
VLRB1 - CL
VLRB1 - CH1 - hinge - CH2 - CH3
VLRB1 - CH1 - hinge - CH2 - CH3-ScFv
VLRB1 - CL
which is a structure of Table 3, structure 3a, wherein each of i
and iv are occupied by the same VLRB domain (i.e., MM3), ii and iii
are occupied by the same VLRB domain (i.e., MM3), vii is occupied
by scFv (i.e., monovalent anti-CD3), and v, vi, and viii are
unoccupied. The CH3/CH3-CH4 domain is a CH3.
FIG. 3B can be represented as
VLRB1 - CL
VLRB1 - CH1 - hinge - CH2 - CH3
scFv - CH1 - hinge - CH2 - CH3
VLRB1 - CL
which is a structure of Table 3, structure 3a, wherein each of i,
ii, and iv are occupied by the same VLRB domain (i.e., MM3), iii is
occupied by scFv (i.e., monovalent anti-CD3), and v-viii are
unoccupied. The CH3/CH3-CH4 domain is a CH3.
FIG. 3C can be represented as
VLRB1 - CH1 - scFv
VLRB1 - CL - hinge - CH2 - CH3
VLRB1 - CH1 - hinge - CH2 - CH3
VLRB1 - CL
which is a structure of Table 3, structure 3b, wherein each of
i, ii, iii, and iv are occupied by the same VLRB domain (i.e., MM3),
v is occupied by scFv (i.e., monovalent anti-CD3), and vi-viii are
unoccupied. The CH3/CH3-CH4 domain is a CH3.
FIG. 3C can be represented as
scFv - CH1
VLRB1 - CL - hinge - CH2 - CH3
VLRB1 - CH1 - hinge - CH2 - CH3
VLRB1 - CL
which is a structure of Table 3, structure 3b, wherein each of
i, ii, and iii are occupied by the same VLRB domain (i.e., MM3), iv is
occupied by scFv (i.e., monovalent anti-CD3), and v-viii are
unoccupied. The CH3/CH3-CH4 domain is a CH3.
FIG. 4 can be represented as
VL1 - CL
VH1 - CH1 - hinge - CH2 - CH3 - VLRB1
VH1 - CH1 - hinge - CH2 - CH3 - VLRB1
VL1 - CL
which is a structure of Table 3, structure 3a wherein each of i
and iv are occupied by the same VL domain (i.e., anti-CD3 VL) and
ii and iii are occupied by the same VH domain (i.e., anti-CD VH). vi
and vii are occupied by the same VLRB domain (i.e., MM3). v and
viii are unoccupied. The CH3/CH3-CH4 domain is a CH3.
FIG. 5 can be represented as
VL1 - CL - VLRB1
VH1 - CH1 - hinge - CH2 - CH3
VH1 - CH1 - hinge - CH2 - CH3
VL1 - CL - VLRB1
which is a structure of Table 3, structure 3a, wherein each of i
and iv are occupied by the same VL domain (i.e., anti-CD3 VL) and
ii and iii are occupied by the same VH domain (i.e., anti-CD VH). v
and viii are occupied by the same VLRB domain (i.e., MM3). vi
and vii are unoccupied. The CH3/CH3-CH4 domain is a CH3.
FIG. 6 can be represented as
VLRB1/2/3/4 - CL - ScFv1/2
VLRB1/2/3/4 - CH1 - hinge - CH2 - CH3
VLRB1/2/3/4 - CH1 - hinge - CH2 - CH3
VLRB1/2/3/4 - CL - ScFv1/2
which is a structure of Table 3, structure 3a, wherein each of
i-iv are occupied by 4 VLRB domains, which may the same or
different. v and viii are occupied by 2 mono-valent ScFv domains,
which may the same or different. vi and vii are unoccupied. The
CH3/CH3-CH4 domain is a CH3.

### D. Exemplary Domains

### 1. VLRB Sequences

The VLRB domain includes at least the antigen binding components of a VLRB antibody. Exemplary VLRB constructions and sequences are provide in, for example U.S. Published Application Nos. 2011/0165584, 2012/0189640, 2017/0081385, and 2017/0008947, each which is specifically incorporated by reference in its entirety.

The structure of VLRB are discussed above and such information there, here, and elsewhere herein can be used to in the disclosed design strategies. The VLRB domain can include an N-terminal leucine rich repeat (LRRNT), one or more leucine rich repeats (LRRs) (referred to herein as the internal LRRs), a C-terminal leucine rich repeat (LRRCT), and a connecting peptide, wherein the connecting peptide comprises an alpha helix. The length of the polypeptide can have as few as about 130 or 137 amino acids or as many as about 225 or 285 amino acids.

Optionally the connecting peptide is located on the N-terminal side of the LRRCT, and more specifically located between the internal LRR and the LRRCT. The connecting peptide can be linked to an internal LRR and the LRRCT. Thus in some embodiments, the VLRB domain includes a LRRNT, one or more internal LRRs, a connecting peptide, and a LRRCT, in that order. In some embodiments, the internal LRR region between the LRRNT and the LRRCT includes 1, 2, 3, 4, 5, 6, 7, 8, or 9 leucine rich repeats, with LRR1 located adjacent to or close to the LRRNT. As used herein LRRs 1, 2, 3, 4, 5, 6, 7, 8, or 9 are considered to run from the LRRNT to the LLRCT consecutively. Thus disclosed domains can include a LRRNT, 1, 1-2, 1-3, 1-4, 1-5, 1-6, 1-7, 1-8, or 1-9 LRRs, a connecting peptide, and a LRRCT, in that order.

Leucine rich repeats (LRRs) are short sequence motifs typically involved in protein to protein interactions, wherein the LRRs comprise multiple leucine residues. LRRs contain leucine or other aliphatic residues, for example, at positions 2, 5, 7, 12, 16, 21, and 24. However, it is understood and herein contemplated that the leucine or other aliphatic residues can occur at other positions in addition to or in the place of residues at positions 2, 5, 7, 12, 16, 21, and 24. For example, a leucine can occur at position 3 rather than position 2. It is also understood that structurally, the motifs form beta-sheet structures. Thus, for example, a disclosed VLRB domains can include a LRRNT, 5 LRR, a LRRCT, and a connecting peptide, and can include 7 beta-sheet structures and the alpha helix of the connecting peptide.

It is understood that the length and sequence of each LRR can vary from the other LRRs in the domain as well as from the LRRNT and LRRCT. For example, in some embodiments, a VLRB domain includes a LRRNT, 1-9 LRR, a connecting peptides, and a LRRCT, wherein the first internal LRR is LRR1, and wherein LRR1 includes less than about 20 amino acids, for example about 18 amino acids. Optionally, the domain further includes LRR2-9, wherein LRR2-9 are less than about 25 amino acids each. In some embodiments, LRR2-9 includes about 24 amino acids each. LRR1-9 can be the same or different from each other in a given domain both in length and in specific amino acid sequence.

The terminal LRRs, designated LRRNT and LRRCT, are typically longer than each internal LRR. The LRRNT and LRRCT include invariant regions (regions that have little variation relative to the rest of the polypeptide as compared to similar variable lymphocyte receptors). The variable regions provide the receptors with specificity, but the invariant regions and general structural similarities across receptors help maintain the protective immunity functions. The domain can include an LRRNT, wherein the LRRNT includes less than about 40 amino acids. Thus, the LRRNT optionally includes the amino acid sequence CPSQCSC (SEQ ID NO:1), CPSRCSC (SEQ ID NO:2), CPAQCSC (SEQ ID NO:3), CPSQCLC (SEQ ID NO:4), CPSQCPC (SEQ ID NO:5), NGATCKK (SEQ ID NO:6), or NEALCKK (SEQ ID NO:7) in the presence or absence of one or more conservative amino acid substitutions. The domains can include a LRRCT, wherein the LRRCT is less than about 60 amino acids, and optionally 40-60 amino acids in length. In some embodiments, the LRRCT includes the sequence KNWIVQHASIVN-(P/L)-X-(S/Y/N/H)-GGVDNVK (SEQ ID NO:8) or KNWIVQHASIVN-(P/L)-XX-(S/Y/N/H)-GGVDNVK (SEQ ID NO:9), where (P/L) means either P or L in that position, X means any amino acid and (S/Y/N/H) means either S, Y, N or H in that position. In particular, specifically disclosed are polypeptides, wherein the LRRCT includes the amino acid sequence TNTPVRAVTEASTSP SKCP (SEQ ID NO:10), SGKPVRSIICP (SEQ ID NO:11), SSKAVLDVTEEEAAEDCV (SEQ ID NO:12), or QSKAVLEITEKDAASDCV (SEQ ID NO:13) in the presence or absence of conservative amino acid substitutions.

As with all peptides, polypeptides, and proteins, it is understood that substitutions in the amino acid sequence of the LRRCT and LRRNT can occur that do not alter the nature or function of the peptides, polypeptides, or proteins. Such substitutions include conservative amino acid substitutions.

The disclosed compositions can also include a connecting peptide. Typically, such peptides are short peptides less than 15 amino acids in length and can form an alpha helix. Thus, for example, specifically disclosed are connecting peptides of 10, 11, 12, 13, 14, and 15 amino acids in length and forming an alpha helix. It is understood that the connecting peptide serves to link structural components of the polypeptide. It is further understood that the connecting peptide of the polypeptide can be linked to the LRRCT.

The polypeptide can include a stalk region. The stalk region typically includes a threonin-proline rich region and is optionally present in the membrane bound form of the polypeptide, along with the GPI anchor and the hydrophobic tail.

Endogenous VLRB antibodies typically include a glycosyl-phosphatidyl-inositol (GPI) anchor which maintains the polypeptide on a membrane surface, and a hydrophobic tail.

Because the disclosed fusion protein are typically preferably soluble, the VLRB domains of the fusion proteins and chimeric antibody constructs can lack one, two, or all three of the stalk, GPI anchor, and hydrophobic tail domains. In some embodiments, VLRB domain lacks a GPI anchor and hydrophobic tail and includes part or all of a stalk domain (e.g., seven amino acids). Such a partial stalk domain can serves as a restriction enzyme site to facilitate VLRB domain substitutions in expression constructs.

The VLRB domains have a desired function. The polypeptide of the VLRB domains as described herein selectively bind an antigen or an agent, much as an antibody selectively binds an antigen or agent. The polypeptides optionally are variable lymphocyte receptors (naturally occurring or non-naturally occurring) or fragments or variants thereof. The term “variable lymphocyte receptors” is used herein in a broad sense and, like the term “antibody” includes various versions having various specificities. The polypeptides can be tested for their desired activity using the in vitro assays described herein, or by analogous methods, after which their therapeutic, diagnostic or other purification activities can be tested according to known testing methods.

The polypeptide can bind an extracellular agent (e.g., a pathogen) or antigen. Agents or antigens can include but are not limited to peptides, polypeptides, lipids, glycolipids, and proteins. Agents or antigens can originate from a variety of sources including but not limited to pathogenic organisms. The binding to an agent or antigen is understood to be selective. By “selectively binding” or “specifically binding” is meant that is binds one agent or antigen to the partial or complete exclusion or other antigens or agents. By “binding” is meant a detectable binding at least about 1.5 times the background of the assay method. For selective or specific binding such a detectable binding can be detected for a given antigen or agent but not a control antigen or agent. Thus, disclosed are VLRB polypeptides that selectively bind, for example, a viral, bacterial, fungal, or protozoan antigen or agent.

Examples of polypeptides that can be used or modified for use as a VLRB antigen binding domain of the disclosed fusion proteins and VLRB-Ig antibodies include the antigen binding domain or full sequence of sequence identifiers 1-43, 45-52, 54, 56, 60-65, 68-72, 75, 77-78, 81-119, 122-125, 129-132, 134-144, and 146-155 of U.S. Published Application No. 2011/0165584, which is specifically incorporated herein in its entirety, including its sequence listing and all of the sequences disclosed therein, and GenBank Accession Numbers AY577941-AY578059 and CK988414-CK988652 each of which is specifically incorporated herein in its entirety, with or preferably without the stalk, GPI anchor, and hydrophobic tail domains. Those sequences including the amino acid sequences of sequence identifiers 1-20 of U.S. Published Application No. 2011/0165584 represent examples of full length VLRs. The sequence including the amino acid sequence of sequence identifier 43 of U.S. Published Application No. 2011/0165584 is an example of a full length VLR with the signal peptide. Each of these published applications is also incorporated by reference in its entirety including its sequence listing.

Other particular VLR antigen binding domains can be derived from:

Sequence identifier 20 of U.S. Published Application No. 2012/0189640, which is specifically incorporated herein in its entirety, including its sequence listing and all of the sequences disclosed therein, provides an antigen specific polypeptide that specifically binds blood group determinant H;

Sequence identifiers 5, 22, 47, 49, 51, 53, 55, 57, 59 or 61 of U.S. Published Application No. 2012/0189640 which is specifically incorporated herein in its entirety, including its sequence listing and all of the sequences disclosed therein, provide an antigen specific polypeptide wherein the binding polypeptide specifically binds a pathogen, such as a Bacillus anthracis cell surface polypeptide, such as BclA;

In some embodiments, the VLRB domain includes or consists of the antigen binding domain of VLRB MM3.

Exemplary MM3 antigen binding domains include
ACPSQCSCPGTDVNCHERRLASVPAEIPTTTKILRLYINQITKLEP GVFD RLTQLTQLGLWDNQLQALPEGVFDRLVNLQKLYLNQNQLLALPVGVFDKLT QLTYLDLNNNQLKSIPRGAFDNLKSLTHIWLYGNPWDCECSDILYLKNWIV QHASIVNPHPYGGVDNVKCSGTNTPVRAVTEASTSPSKCPG (SEQ ID NO:14) (which is the polypeptide sequence of sequence identifier 56 of U.S. Published Application No. 2019/0202887, which is specifically incorporated herein in its entirety, including its sequence listing and all of the sequences disclosed therein), or
ACPSQCSCPGTDVNCHERRLASVPAEIPTTTKILRLYINQITKLEP GVFDRLTQLTQLGLWDNQLQALPEGVFDRLVNLQKLYLNQNQLLALPVGVF DKLTQLTYLDLNNNQLKSIPRGAFDNLKSLTHIWLYGNPWDCECSDILYLK NWIVQHASIVNPHPYGGVDNVKCSGTNTPVRAVTEASTSPSKCPGYVATTT (SEQ ID NO:15, which including the MM3 antigen binding domain and part of the stalk);
or a humanized antigen binding domain thereof such as
CPAACTCSNNIVDCRGKGLTEIPTNLPETITEIRLEQNTIKVIPPG AFSP YKKLRILRLYINQISELAPDAFQGLRSLNQLVLWDNKITELPKSLFEGLFS LQLLYLNANKINCLRVDAFQDLHNLNYLDLNNNKLQTIAKGTFSPLRAIQH IWLYGNPFICDCHLKWLADYLHASIVNPHPYGGVDNARCTSPRRLAN(SEQ ID NO:16) (which is the polypeptide sequence of sequence identifier 64 of U.S. Published Application No. 2019/0202887)
CPAACTCSNNIVDCRGKGLTEIPTNLPETITEIRLEQNTIKVIPPG AFSP YKKLRILRLYINQISELAPDAFQGLRSLNQLVLWDNKITELPKSLFEGLFS LQLLYLNANKINCLRVDAFQDLHNLNYLDLNNNKLQTIAKGTFSPLRAIQH IWLYGNPFICDCHLKWLADYLHHASIVNPHPYGGVDNARCTSPRRLAN (SEQ ID NO:17) (which is the polypeptide sequence of sequence identifier 74 of U.S. Published Application No. 2019/0202887);
CPAACTCSNNIVDCRGKGLTEIPTNLPETITEIRLEQNTIKVIPPG AFSP YKKLRILRLYINQISELAPDAFQGLRSLNQLVLWDNKITELPKSLFEGLFS LQLLYLNANKINCLRVDAFQDLHNLNYLDLNNNKLQTIAKGTFSPLRAIQH IWLYGNPFICDCHLKWLADYLHTHASIVNPHPYGGVDNRCTSPRRLAN (SEQ ID NO:18) (which is the polypeptide sequence of sequence identifier 75 of U.S. Published Application No. 2019/0202887);
CPAACTCSNNIVDCRGKGLTEIPTNLPETITEIRLEQNTIKVIPPG AFSP YKKLRILRLYINQISELAPDAFQGLRSLNQLVLWDNKITELPKSLFEGLFS LQLLYLNANKINCLRVDAFQDLHNLNYLDLNNNKLQTIAKGTFSPLRAIQH IWLYGNPFICDCHLKWLADYLHTNHASIVNPHPYGGVDNARCTSPRRLAN (SEQ ID NO:19) (which is the polypeptide sequence of sequence identifier 76 of U.S. Published Application No. 2019/0202887);
CPAACTCSNNIVDCRGKGLTEIPTNLPETITEIRLEQNTIKVIPPG AFSP YKKLRILRLYINQISELAPDAFQGLRSLNQLVLWDNKITELPKSLFEGLFS LQLLYLNANKINCLRVDAFQDLHNLNYLDLNNNKLQTIAKGTFSPLRAIQH IWLYGNPFICDCHLKWLADYLHTNPHASIVNPHPYGGVDNRCTSPRRLAN (SEQ ID NO:20) (which is the polypeptide sequence of sequence identifier 77 of U.S. Published Application No. 2019/0202887);
or a variant thereof with, e.g., 80, 85, 90, 95, 97, 98% percent identity or similarity thereto.

Based on the structures and function taught herein and otherwise known in the art, it will be understood that these sequences are examples of a genus of polypeptides. It is understood that disclosed are full length VLRs and fragments thereof that can used as the VLRB domain(s) of the disclosed fusion proteins and VLRB-Ig antibodies.

Other examples of VLRB antibodies and antigen binding domains are disclosed in U.S. Published Application Nos. 2020/0308247, 2019/0256574, 2019/0202897, 2019/0202887, 2017/0081385, 2017/0008947, 2016/0376348, 2012/0189640, 2012/0107929, and 2011/0165584, each of which is specifically incorporated by reference herein in its entirety. Any of the VLRB domains can be humanized, for example, as discussed in, for example, U.S. Published Application No. 2019/0202887. Thus, in some embodiments, the VLRB domain is a humanized VLRB antigen binding domain.

Methods of making antigen specific proteins having a selected antigen specificity are also known in the art, and can be used to prepared VLR antigen binding domains of the disclosed fusion proteins and chimeric antibodies. The methods are described in, e.g., U.S. Published Application No. 2012/0189640, and can include administering to a lamprey or hagfish one or more target antigens (e.g., a target carbohydrate, a target protein, a target pathogen, a target glycoprotein, a target lipid, a target glycolipid, target tumor antigen, target ligand, target receptor, target cell or any combination thereof including, for example, two carbohydrates, one carbohydrate and one protein, etc.); isolating an antigen specific protein-encoding RNA from lymphocytes of the lamprey or hagfish; amplifying antigen specific protein encoding cDNA from the isolated RNA; cloning the cDNA into an expression vector; expressing the expression vector in a bacterium transformed with the expression vector; isolating a cDNA clone; transfecting a cultured cell with the cDNA clone; screening the culture supernatant for an ability to bind the target antigen, and isolating the antigen specific protein from the supernatant that binds the target antigen.

In other embodiments, methods of making may include preparation of total RNA, preparation cDNA with olig-dT primer, then amplification of the cDNA with VLRB gene-specific primers that also contain regions of vector sequence complementarity to facilitate cloning.

A transfection/screening process referred to as Transfectoma can be used, but is low throughput, few hundred transfectants screened, and has largely been supplanted by yeast display and phage/phagemid display, millions (yeast) or 100s of millions (phage) screened. For VLRB phage display, see, e.g., Hassan, et al., “Generation of lamprey monoclonal antibodies (“Lambribodies”) using a phage display system,” Biomolecules, 9, 868 (2019); doi:10.3390/biom9120868; and for yeast display, see Tasumi, et al., “High-affinity lamprey VLRA and VLRB monoclonal antibodies,” Proc. Natl. Acad. Sci. USA, 106, 12891-12896 (2009), each of which is specifically incorporated by reference herein in its entirety.

Another exemplary VLRB antigen binding domain is ACPSQCSCSGTTVNCKSKSLASVPAGIPTTTRVLYLNDNQITKLEPGVFDR LVNLQTLWLNNNQLTSLPAGLFDSLTQLTILALDSNQLQALPVGVFGRLVD LQQLYLGSNQLSALPSAVFDRLVHLKELLMCCNKLTELPRGIERLTHLTHL ALDQNQLKSIPHGAFDRLSSLTHAYLFGNPWDCECRDIMYLRNWVADHTSI VMRWDGKAVNDPDSAKCAGTNTPVRAVTEASTSPSKCPGYVATTT (SEQ ID NO:21, O13 VLRB antigen binding domain)

### 2. Antibody Variable and Receptor/Ligand Domains

Exemplary antibodies from which VH, VL, ScFv, and VHH antigen binding domains can be utilized in the disclosed fusion proteins and VLRB antibodies are provided. Such antibodies include, but are not limited to, daratumumab (DARZALEX®) as well as those discussed in Reichert, Mabs, 3(1): 76-99 (2011), for example, AIN-457, bapineuzumab, brentuximab vedotin, briakinumab, dalotuzumab, epratuzumab, farletuzumab, girentuximab (WX-G250), naptumomab estafenatox, necitumumab, obinutuzumab, otelixizumab, pagibaximab, pertuzumab, ramucirumab, REGN88, reslizumab, solanezumab, Tlh, teplizumab, trastuzumab emtansine, tremelimumab, vedolizumab, zalutumumab and zanolimumab, and elsewhere including, but not limited to, rituximab (Rituxan®, IDEC/Genentech/Roche) (see for example U.S. Pat. No. 5,736,137), a chimeric anti-CD20 antibody approved to treat Non-Hodgkin's lymphoma; HuMax-CD20, an anti-CD20 currently being developed by Genmab, an anti-CD20 antibody described in U.S. Pat. No. 5,500,362, AME-133 (Applied Molecular Evolution), hA20 (Immunomedics, Inc.), HumaLYM (Intracel), and PR070769 (PCT/US2003/040426, entitled “Immunoglobulin Variants and Uses Thereof”), trastuzumab (Herceptin®, Genentech) (see for example U.S. Pat. No. 5,677,171), a humanized anti-Her2/neu antibody approved to treat breast cancer; pertuzumab (rhuMab-2C4, Omnitarge), currently being developed by Genentech; an anti-Her2 antibody described in U.S. Pat. No. 4,753,894; cetuximab (Erbitux®, Imclone) (U.S. Pat. No. 4,943,533; PCT WO 96/40210), a chimeric anti-EGFR antibody in clinical trials for a variety of cancers; ABX-EGF (U.S. Pat. No. 6,235,883), currently being developed by Abgenix-Immunex-Amgen; HuMax-EGFr (U.S. Ser. No. 10/172,317), currently being developed by Genmab; 425, EMD55900, EMD62000, and EMD72000 (Merck KGaA) (U.S. Pat. No. 5,558,864; Murthy et al. 1987, Arch Biochem Biophys. 252(2):549-60; Rodeck et al., 1987, J Cell Biochem. 35(4):315-20; Kettleborough et al., 1991, Protein Eng. 4(7):773-83); 1CR62 (Institute of Cancer Research) (PCT WO 95/20045; Modjtahedi et al., 1993, J. Cell Biophys. 1993, 22(1-3):129-46; Modjtahedi et al., 1993, Br J Cancer. 1993, 67(2):247-53; Modjtahedi et al, 1996, Br J Cancer, 73(2):228-35; Modjtahedi et al, 2003, Int J Cancer, 105(2):273-80); TheraCIM hR3 (YM Biosciences, Canada and Centro de Immunologia Molecular, Cuba (U.S. Pat. Nos. 5,891,996; 6,506,883; Mateo et al, 1997, Immunotechnology, 3(1):71-81); mAb-806 (Ludwig Institute for Cancer Research, Memorial Sloan-Kettering) (Jungbluth et al. 2003, Proc Natl Acad Sci USA. 100(2):639-44); KSB-102 (KS Biomedix); MRI-1 (IVAX, National Cancer Institute) (PCT WO 0162931A2); and SC100 (Scancell) (PCT WO 01/88138); alemtuzumab (Campath®, Millenium), a humanized mAb currently approved for treatment of B-cell chronic lymphocytic leukemia; muromonab-CD3 (Orthoclone OKT3®), an anti-CD3 antibody developed by Ortho Biotech/Johnson & Johnson, ibritumomab tiuxetan (Zevalin®), an anti-CD20 antibody developed by IDEC/Schering AG, gemtuzumab ozogamicin (Mylotarg®), an anti-CD33 (p67 protein) antibody developed by Celltech/Wyeth, alefacept (Amcvive®), anti-LFA-3 Fc fusion developed by Biogen), abciximab (ReoPro®), developed by Centocor/Lilly, basiliximab (Simulect®), developed by Novartis, palivizumab (Synagis®), developed by Medimmune, infliximab (Remicade®), an anti-TNFalpha antibody developed by Centocor, adalimumab (Humira®), an anti-TNFalpha antibody developed by Abbott, Humicade®, an anti-TNFalpha antibody developed by Celltech, golimumab (CNTO-148), a fully human TNF antibody developed by Centocor, etanercept (Enbrel®), an p75 TNF receptor Fc fusion developed by Immunex/Amgen, lenercept, an p55TNF receptor Fc fusion previously developed by Roche, ABX-CBL, an anti-CD 147 antibody being developed by Abgenix, ABX-IL8, an anti-IL8 antibody being developed by Abgenix, ABX-MAI, an anti-MUC18 antibody being developed by Abgenix, Pemtumomab (R1549,90Y-muHMFG1), an anti-MUC1 in development by Antisoma, Therex (R1550), an anti-MUC1 antibody being developed by Antisoma, AngioMab (AS1405), being developed by Antisoma, HuBC-1, being developed by Antisoma, Thioplatin (AS1407) being developed by Antisoma, Antegrene (natalizumab), an anti-alpha-4-beta-1 (VLA-4) and alpha-4-beta-7 antibody being developed by Biogen, VLA-1 mAb, an anti-VLA-1 integrin antibody being developed by Biogen, LTBR mAb, an anti-lymphotoxin beta receptor (LTBR) antibody being developed by Biogen, CAT-152, an anti-TGF-.beta.2 antibody being developed by Cambridge Antibody Technology, ABT 874 (J695), an anti-IL-12 p40 antibody being developed by Abbott, CAT-192, an anti-TGF.beta.1 antibody being developed by Cambridge Antibody Technology and Genzyme, CAT-213, an anti-Eotaxinl antibody being developed by Cambridge Antibody Technology, LyntphoStat-B® an anti-Blys antibody being developed by Cambridge Antibody Technology and Human Genome Sciences Inc., TRAIL-R1mAb, an anti-TRAIL-R1 antibody being developed by Cambridge Antibody Technology and Human Genome Sciences, Inc. Avastin® bevacizumab, rhuMAb-VEGF), an anti-VEGF antibody being developed by Genentech, an anti-HER receptor family antibody being developed by Genentech, Anti-Tissue Factor (ATF), an anti-Tissue Factor antibody being developed by Genentech. Xolair® (Omalizurnab), an anti-IgE antibody being developed by Genentech, Raptiva® (Efalizurnab), an anti-CD11a antibody being developed by Genentech and Xoma, MLN-02 Antibody (formerly LDP-02), being developed by Genentech and Millenium Pharmaceuticals, HuMax CD4, an anti-CD4 antibody being developed by Genmab, HuMax-IL15, an anti-IL15 antibody being developed by Genmab and Amgen, HuMax-Inflam, being developed by Genmab and Medarex, HuMax-Cancer, an anti-Heparanase I antibody being developed by Genmab and Medarex and Oxford GcoSciences, HuMax-Lymphoma, being developed by Genmab and Amgen, HuMax-TAC, being developed by Genmab, IDEC-131, and anti-CD40L antibody being developed by IDEC Pharmaceuticals, IDEC-151 (Clenoliximab), an anti-CD4 antibody being developed by IDEC Pharmaceuticals, IDEC-114, an anti-CD80 antibody being developed by IDFC Pharmaceuticals, IDEC-152, an anti-CD23 being developed by IDEC Pharmaceuticals, anti-macrophage migration factor (MIF) antibodies being developed by IDEC Pharmaceuticals, BEC2, an anti-idiotypic antibody being developed by Imclone, IMC-1C11, an anti-KDR antibody being developed by Imclone, DC101, an anti-flk-1 antibody being developed by Imclone, anti-VE cadherin antibodies being developed by Imclone, CEA-Cide® (labetuzumab), an anti-carcinoembryonic antigen (CEA) antibody being developed by Immunomedics, LymphoCide® (Epratuzumab), an anti-CD22 antibody being developed by Immunomedics, AFP-Cide, being developed by Immunomedics, MyelomaCide, being developed by Immunomedics, LkoCide, being developed by Immunomedics, ProstaCide, being developed by Immunomedics, MDX-010, an anti-CTLA4 antibody being developed by Medarex, MDX-060, an anti-CD30 antibody being developed by Medarex, MDX-070 being developed by Medarex, MDX-018 being developed by Medarex, Osidem® (IDM-I), and anti-Her2 antibody being developed by Medarex and Immuno-Designed Molecules, HuMaxe-CD4, an anti-CD4 antibody being developed by Medarex and Genmab, HuMax-IL15, an anti-IL15 antibody being developed by Medarex and Genmab, CNTO 148, an anti-TNFα antibody being developed by Medarex and Centocor/J&J. CNTO 1275, an anti-cytokine antibody being developed by Centocor/J&J, MOR101 and MOR102, anti-intercellular adhesion molecule-1 (ICAM-1) (CD54) antibodies being developed by MorphoSys, MOR201, an anti-fibroblast growth factor receptor 3 (FGFR-3) antibody being developed by MorphoSys, Nuvion® (visilizumab), an anti-CD3 antibody being developed by Protein Design Labs, HuZAFO, an anti-gamma interferon antibody being developed by Protein Design Labs, Anti-α5β1 Integrin, being developed by Protein Design Labs, anti-IL-12, being developed by Protein Design Labs, ING-1, an anti-Ep-CAM antibody being developed by Xoma, Xolair® (Omalizumab) a humanized anti-IgE antibody developed by Genentech and Novartis, and MLNO1, an anti-Beta2 integrin antibody being developed by Xoma. In another embodiment, the therapeutics include KRN330 (Kirin); huA 33 antibody (A33, Ludwig Institute for Cancer Research); CNTO 95 (alpha V integrins, Centocor); MEDI-522 (alpha V133 integrin, Medimmune); volociximab (αVβ1 integrin, Biogen/PDL); Human mAb 216 (B cell glycosolated epitope, NCI); BiTE MT103 (bispecific CD19× CD3, Medimmune); 4G7×H22 (Bispecific BcellxFcgammaRl, Meclarex/Merck KGa); rM28 (Bispecific CD28×MAPG, U.S. Patent No. EP1444268); MDX447 (EMD 82633) (Bispecific CD64×EGFR, Medarex); Catumaxomab (removah) (Bispecific EpCAM×anti-CD3, Trion/Fres); Ertumaxomab (bispecific HER2/CD3, Fresenius Biotech); oregovomab (OvaRex) (CA-125, ViRexx); Rencarex® (WX G250) (carbonic anhydrase IX, Wilex); CNTO 888 (CCL2, Centocor); TRC105 (CD105 (endoglin), Tracon); BMS-663513 (CD137 agonist, Brystol Myers Squibb); MDX-1342 (CD19, Medarex); Siplizumab (MEDI-507) (CD2, Medimmune); Ofatumumab (Humax-CD20) (CD20, Genmab); Rituximab (Rituxan) (CD20, Genentech); THIOMAB (Genentech); veltuzumab (hA20) (CD20, Immunomedics); Epratuzumab (CD22, Amgen); lumiliximab (IDEC 152) (CD23, Biogen); muromonab-CD3 (CD3, Ortho); HuM291 (CD3 fc receptor, PDL Biopharma); HeFi-1, CD30, NCI); MDX-060 (CD30, Medarex); MDX-1401 (CD30, Medarex); SGN-30 (CD30, Seattle Genetics); SGN-33 (Lintuzumab) (CD33, Seattle Genetics); Zanolimumab (HuMax-CD4) (CD4, Genmab); HCD 122 (CD40, Novartis); SGN-40 (CD40, Seattle Genetics); Campathlh (Alemtuzumab) (CD52, Genzyme); MDX-1411 (CD70, Medarex); hLL1 (EPB-I) (CD74.38, Immunomedics); Galiximab (IDEC-144) (CD80, Biogen); MT293 (TRC093/D93) (cleaved collagen, Tracon); HuLuc63 (CS1, PDL Pharma); ipilimumab (MDX-010) (CTLA4, Brystol Myers Squibb); Tremelimumab (Ticilimumab, CP-675,2) (CTLA4, Pfizer); 1-IGS-ETR1 (Mapatumumab) (DR4TRAIL-R1 agonist, Human Genome Science/Glaxo Smith Kline); AMG-655 (DR5, Amgen); Apomab (DR5, Genentech); CS-1008 (DR5, Daiichi Sankyo); HGS-ETR2 (lexatumumab) (DR5TRAIL-R2 agonist, HGS); Cetuximab (Erbitux) (EGFR, Imclone); IMC-11F8, (EGFR, Imclone); Nimotuzumab (EGFR, YM Bio); Panitumumab (Vectabix) (EGFR, Amgen); Zalutumumab (HuMaxEGFr) (EGFR, Genmab); CDX-110 (EGFRvIII, AVANT Immunotherapeutics); adecatumumab (MT201) (Epcam, Merck); edrecolomab (Panorex, 17-1A) (Epcam Glaxo/Centocor); MORAb-003 (folate receptor a, Morphotech); KW-2871 (ganglioside GD3, Kyowa); MORAb-009 (GP-9, Morphotech); CDX-1307 (MDX-1307) (hCGb, Celldex); Trastuzumab (Herceptin) (HER2, Celldex); Pertuzumab (rhuMAb 2C4) (HER2 (DI), Genentech); apolizumab (HLA-DR beta chain, PDL Pharma); AMG-479 (IGF-1R, Amgen); anti-IGF-1R R1507 (IGF1-R, Roche); CP 751871 (IGF 1-R, Pfizer); IMC-A12 (IGF1-R, Imclone); B1111022 Biogen); Mik-beta-1 (IL-2Rb (CD122), Hoffman LaRoche); CNTO 328 (IL6, Centocor); Anti-KIR (1-7F9) (Killer cell Ig-like Receptor (KIR), Novo); Hu3S193 (Lewis (y), Wyeth, Ludwig Institute of Cancer Research); hCBE-11 (LTβR, Biogen); HuHMFG1 (MUC1, Antisoma/NCI); RAV 12 (N-linked carbohydrate epitope, Raven); CAL (parathyroid hormone-related protein (PTH-rP), University of California); CT-011 (PD1, CtireTech); MDX-1106 (ono-4538) (PDL Nileclarox/Ono); MAb CT-011 (PD1, Curetech); IMC-3G3 (PDGFRa, Imclone); bavituximab (phosphatidylserine, Peregrine); huJ591 (PSMA, Cornell Research Foundation); muJ591 (PSMA, Cornell Research Foundation); GC1008 (TGFb (pan) inhibitor (IgG4), Genzyme); Infliximab (Remicade) (TNFα, Centocor); A27.15 (transferrin receptor, Salk Institute, INSERN WO 2005/111082); E2.3 (transferrin receptor, Salk Institute); Bevacizumab (Avastin) (VEGF, Genentech); HuMV833 (VEGF, Tsukuba Research Lab-WO/2000/034337, University of Texas); IMC-18F1 (VEGFR1, Imclone); and IMC-1121 (VEGFR2, Imclone).

Other antibodies include bispecific T cell engagers, in which one or both of the antigen binding domains are incorporated in the VLRB-Ig as discussed herein. Examples of suitable bispecific antibodies and other antibodies are discussed in Tian, et al., “Bispecific T cell engagers: an emerging therapy for management of hematologic malignancies”, Journal of Hematology & Oncology volume 14, Article number: 75 (2021), but are not limited to those of Table 5:

TABLE 5
Exemplary antibodies

| Disease | Target | Name | Antibody format |
|---|---|---|---|
| AML | CD123-CD3 | MGD006 | DART |
| | | XmAb14045 | XmAb |
| | CD33-CD3 | AMG 330 | BiTE |
| | | AMV 564 | TandAb |
| | FLT3-CD3 | 7370 | BiTE |
| | CLEC12A-CD3 | MCLA-117 | Biclonics |
| | WT1-CD3 | ESK1-BiTE | BiTE |
| ALL | CD19-CD3 | Blinatumomab | BiTE |
| | | AFM11 | TandAb |
| MM | BCMA-CD3 | AMG420 | BiTE |
| | | AMG701 | BiTE-Fc |
| | GPRC5D-CD3 | Talquetamab | DuoBody |
| | CD38-CD3 | AMG424 | XmAb |
| | | Bi38-3 | BiTE |
| | FCRL5-CD3 | anti-FcRH5/CD3 | Knobs-into-holes |
| | | TDB | |
| NHL | CD19-CD3 | Blinatumomab | BiTE |
| | CD20-CD3 | REGN1979 | Veloci-Bi platform |
| | | Mosunetuzumab | Knobs-into-holes |
| | | RG6026 | 2:1 CrossMab |
| MDS | CD33-CD3 | AMV564 | TandAb |
| | CD123-CD3 | MGD006 | DART |

Exemplary non-VLRB antigen binding domains are DVQLVQSGAEVKKPGASVKVSCKASGYTFTRYTMHWVRQAPGQGLEWIGYI NPSRGYTNYADSVKGRFTITTDKSTSTAYMELSSLRSEDTATYYCARYYDD HYCLDYWGQGTTVTVSSGEGTSTGSGGSGGSGGADDIVLTQSPATLSLSPG ERATLSCRASQSVSYMNWYQQKPGKAPKRWIYDTSKVASGVPARFSGSGSG TDYSLTINSLEAEDAATYYCQQWSSNPLTFGGGTKVEIKS (SEQ ID NO:22), which is low affinity TR66 anti-CD3 scFv (KD=100 nM), used in the experiments below (see also WO 2007/073499A2, which is specifically incorporated by reference herein in its entirety), and EVQLVESGGGLVQPGGSLRLSCAASGFTFSTYAMNWVRQAPGKGLEWVGRI RSKYNNYATYYADSVKGRFTISRDDSKNTLYLQMNSLRAEDTAVYYCVRHG NFGDSYVSWFAYWGQGTLVTVSSgkpgsgkpgsgkpgsgkpgsQAVVTQEP SLTVSPGGTVTLTCGSSTGAVTTSNYANWVQQKPGKSPRGLIGGTNKRAPG VPARFSGSLLGGKAALTISGAQPEDEADYYCALWYSNHWVFGGGTKLTVL (SEQ ID NO:34), where the CDRs are identified with bold and the linker is shown in lowercase, which is an anti-CD3 high affinity VH-L-VL scFv (see also WO 2017/091656, which is specifically incorporated by reference herein in its entirety).

In some embodiments, the fusion proteins include a R/L domain that binds to a cell surface receptor or ligand that is specifically expressed on immune cells, tumor cells or tumor-associated neovasculature or are overexpressed on tumor cells or tumor-associated neovasculature as compared to normal tissue. Tumors also secrete a large number of ligands into the tumor microenvironment that affect tumor growth and development. Thus, the R/L can be a receptor(s) that bind to a ligand typically expressed on the surface of cells, for example immune cells, for example members of the histocompatibility antigen family, or on the surface of tumors, including, but not limited to growth factors, cytokines and chemokines. In other embodiments the R/L is a ligand that binds to a receptor expressed on the surface of immune cells or tumors.

Thus, in some embodiments, fusion proteins contain a domain that specifically binds to a chemokine or a chemokine receptor. Chemokines are soluble, small molecular weight (8-14 kDa) proteins that bind to their cognate G-protein coupled receptors (GPCRs) to elicit a cellular response, usually directional migration or chemotaxis. Tumor cells secrete and respond to chemokines, which facilitate growth that is achieved by increased endothelial cell recruitment and angiogenesis, subversion of immunological surveillance and maneuvering of the tumoral leukocyte profile to skew it such that the chemokine release enables the tumor growth and metastasis to distant sites. Thus, chemokines are vital for tumor progression.

Based on the positioning of the conserved two N-terminal cysteine residues of the chemokines, they are classified into four groups namely CXC, CC, CX3C and C chemokines. The CXC chemokines can be further classified into ELR+ and ELR- chemokines based on the presence or absence of the motif ‘glu-leu-arg (ELR motif)’ preceding the CXC sequence. The CXC chemokines bind to and activate their cognate chemokine receptors on neutrophils, lymphocytes, endothelial and epithelial cells. The CC chemokines act on several subsets of dendritic cells, lymphocytes, macrophages, eosinophils, natural killer cells but do not stimulate neutrophils as they lack CC chemokine receptors except murine neutrophils. There are approximately 50 chemokines and only 20 chemokine receptors, thus there is considerable redundancy in this system of ligand/receptor interaction.

Chemokines elaborated from the tumor and the stromal cells bind to the chemokine receptors present on the tumor and the stromal cells. The autocrine loop of the tumor cells and the paracrine stimulatory loop between the tumor and the stromal cells facilitate the progression of the tumor. Notably, CXCR2, CXCR4, CCR2 and CCR7 play major roles in tumorigenesis and metastasis. CXCR2 plays a vital role in angiogenesis and CCR2 plays a role in the recruitment of macrophages into the tumor microenvironment. CCR7 is involved in metastasis of the tumor cells into the sentinel lymph nodes as the lymph nodes have the ligand for CCR7, CCL21. CXCR4 is mainly involved in the metastatic spread of a wide variety of tumors.

### 3. Constant Domains

In certain embodiments, the CHL, CH1, hinge, CH2, CH3, and/or CH4 are mammalian sequences, including, but not limited to mouse, rat, hamster, rabbit, camel, donkey, goat, and human sequences. They can be endogenous sequences to the subject to whom the chimeric antibody is administered. In a preferred embodiment, the sequences are human sequences. In certain embodiments, the CHL, CH1, CH2, and/or CH3 sequences are from an IgA1, IgA2, IgD, IgE, IgM, IgG1, IgG2, IgG3, or IgG4 isotype, and/or CH4 sequences are from an IgE or IgM isotype. Any of the CHL, CH1, hinge, CH2, CH3, and/or CH4 sequences can be a naturally occurring sequence, or a variant thereof with at least 70, 75, 80, 85, 90, 95, or more sequence identity thereto. Preferred variants are orthogonal mutations that improve the activity or performance of one or more of the constant domains, or the chimeric antibody as a whole or alternatively, eliminate an activity such as binding to one or more FcRs or activation of complement.

The CL amino acid sequences can be lambda light chain constant domain sequences. In particular embodiments, the CL amino acid sequences are human lambda light chain constant domain sequences, such as the lambda light chain sequence of UniProt accession number POCG04 which is specifically incorporated herein in its entirety. GQPKANPTVTLFPPSSEELQANKATLVCLISDFYPGAVTVAWKADGSPVKA GVETTKPSKQSNNKYAASSYLSLTPEQWKSHRSYSCQVTHEGSTVEKTVAP TECS (SEQ ID NO:23) is the polypeptide sequence of P0CG04. IGLC1_HUMAN, Immunoglobulin lambda constant 1. Thus, in some embodiments, the CL domain is SEQ ID NO:23, or a fragment or variant thereof with at least 75%, 80%, 85%, 90%, 95%, 96%, 97%, 98%, or 99% sequence identity thereto.

The CL amino acid sequences can be kappa light chain constant domain sequences. In a preferred embodiment, the CL amino acid sequences are human kappa light chain constant domain sequences, such as the kappa light chain sequence is UniProt accession number P01834 which is specifically incorporated herein in its entirety. RTVAAPSVFIFPPSDEQLKSGTASVVCLLNNFYPREAKVQWKVDNALQSGN SQESVTEQDSKDSTYSLSSTLTLSKADYEKHKVYACEVTHQGLSSPVTKSF NRGEC (SEQ ID NO:24) is the polypeptide sequence of P01834. IGKC_HUMAN Immunoglobulin kappa constant. Thus, in some embodiments, the CL domain is SEQ ID NO:24, or a fragment or variant thereof with at least 75%, 80%, 85%, 90%, 95%, 96%, 97%, 98%, or 99% sequence identity thereto.

In some embodiments, one of more of the heavy chain domains sequences are derived from the human IgG1 sequence of UniProt accession number or PODOX5 or P01857, each of which is specifically incorporated by reference herein in their entireties, the sequences of which are provided below: QVQLVQSGGGVVQPGRSLRLSCAASGFTFSRYTIHWVRQAPGKGLEWVAVM SYNGNNKHYADSVNGRFTISRNDSKNTLYLNMNSLRPEDTAVYYCARIRDT AMFFAHWGQGTLVTVSSASTKGPSVFPLAPSSKSTSGGTAALGCLVKDYFP EPVTVSWNSGALTSGVHTFPAVLQSSGLYSLSSVVTVPSSSLGTQTYICNV NHKPSNTKVDKKVEPKSCDKTHTCPPCPAPELLGGPSVFLFPPKPKDTLMI SRTPEVTCVVVDVSHEDPEVKFNWYVDGVEVHNAKTKPREEQYNSTYRVVS VLTVLHQDWLNGKEYKCKVSNKALPAPIEKTISKAKGQPREPQVYTLPPSR DELTKNQVSLTCLVKGFYPSDIAVEWESNGQPENNYKTTPPVLDSDGSFFL YSKLTVDKSRWQQGNVFSCSVMHEALHNHYTQKSLSLSPGK (SEQ ID NO:25) is the polypeptide sequence of PODOX5·IGG1_HUMAN, Immunoglobulin gamma-1 heavy chain, which also provides the following domain/region annotations:

TABLE 6
P0DOX5 sequence annotations

| TYPE ID | POSITION(S) | DESCRIPTION |
|---|---|---|
| Domain | 1-96 | Ig-like |
| Region | 1-119 | Variable (V) domain, |
| | | in antigen recognition |
| Region | 120-449 | Constant (C) domain |
| Domain | 125-218 | Ig-like 2 |
| Domain | 240-339 | Ig-like 3 |
| Domain | 348-444 | Ig-like 4 |

ASTKGPSVFPLAPSSKSTSGGTAALGCLVKDYFPEPVTVSWNSGALTSGVH TFPAVLQSSGLYSLSSVVTVPSSSLGTQTYICNVNHKPSNTKVDKKVEPKS CDKTHTCPPCPAPELLGGPSVFLFPPKPKDTLMISRTPEVTCVVVDVSHED PEVKFNWYVDGVEVHNAKTKPREEQYNSTYRVVSVLTVLHQDWLNGKEYKC KVSNKALPAPIEKTISKAKGQPREPQVYTLPPSRDELTKNQVSLTCLVKGF YPSDIAVEWESNGQPENNYKTTPPVLDSDGSFFLYSKLTVDKSRWQQGNVF SCSVMHEALHNHYTQKSLSLSPGK (SEQ ID NO:26), the polypeptide sequence of P01857·IGHG1_HUMAN, Immunoglobulin heavy constant gamma 1, which also provides the following domain/region annotations:

TABLE 7
P01857 sequence annotations:

| TYPE ID | POSITION(S) | DESCRIPTION |
|---|---|---|
| Region | 1-21 | Disordered |
| Region | 1-98 | CH1 |
| Domain | 6-99 | Ig-like 1 |
| Region | 99-110 | Hinge |
| Region | 111-223 | CH2 |
| Domain | 121-220 | Ig-like 2 |
| Region | 224-330 | CH3 |
| Domain | 229-325 | Ig-like 3 |

SEQ ID NO:25, PODOX5·IGG1_HUMAN, Immunoglobulin gamma-1 heavy chain and SEQ ID NO:26, P01857·IGHG1_HUMAN, Immunoglobulin heavy constant gamma 1 are related with SEQ ID NO:26 lacking the Variable (V) domain, involved in antigen recognition of SEQ ID NO:25. This variable domain of SEQ ID NO:25 not typically utilized in the disclosed constructs (e.g., absent or substituted). Exemplary mutations sites, some of which are discussed in more detail below, are illustrated with bold in SEQ ID NOS:25 and 26.

Thus in some embodiments, the fusion proteins include one or more of the CH1, hinge, CH2, and/or CH3 region(s) of SEQ ID NO:26 or the corresponding sequence of SEQ ID NO:25, or a fragment or variant thereof with at least 75%, 80%, 85%, 90%, 95%, 96%, 97%, 98%, or 99% sequence identity thereto.

In some embodiments, the fusion proteins include SEQ ID NO:26 preferably without the variable domain (e.g., amino acids 120-444) or the corresponding sequence of SEQ ID NO:25, or a fragment or variant thereof with at least 75%, 80%, 85%, 90%, 95%, 96%, 97%, 98%, or 99% sequence identity thereto. In some embodiments, the variant includes mutations at one or more of the bolded residues, optionally wherein one or more of the mutations is discussed in more detail below.

In an exemplary embodiment, the CH1 sequences are from an IgG1 isotype. In a specific example, the CH1 sequence is UniProt accession number P01857 (SEQ ID NO:26), amino acids 1-98: ASTKGPSVFPLAPSSKSTSGGTAALGCLVKDYFPEPVTVSWNSGALTSGVH TFPAVLQSSGLYSLSSVVTVPSSSLGTQTYICNVNHKPSNTKVDKKV (SEQ ID NO:57), or a fragment or variant thereof with at least 75%, 80%, 85%, 90%, 95%, 96%, 97%, 98%, or 99% sequence identity thereto.

In an exemplary embodiment, the hinge sequences are from an IgG1 isotype. In a specific example, the CH1 sequence is UniProt accession number P01857 (SEQ ID NO:26), amino acids 99-110 EPKSCDKTHTCP (SEQ ID NO:58), or a fragment or variant thereof with at least 75%, 80%, 85%, 90%, 95%, 96%, 97%, 98%, or 99% sequence identity thereto.

In certain embodiments, the CH1 sequence and the CL sequences separately include respectively orthogonal modifications in endogenous CH1 and CL sequences, such as those that introduce engineered disulfide bridges, charged-pair mutations, or a combination thereof. See, e.g., U.S. Pat. Nos. 8,053,562, 9,527,927, 8,592,562, 9,248,182, and 9,358,286 each of which is incorporated herein by reference herein in its entirety.

An exemplary CH2 sequence is the amino acid sequence of UniProt accession number P01857 (SEQ ID NO:26), amino acids 111-223: PCPAPELLGGPSVFLFPPKPKDTLMISRTPEVTCVVVDVSHEDPEVKFNWY VDGVEVHNAKTKPREEQYNSTYRVVSVLTVLHQDWLNGKEYKCKVSNKALP APIEKTISKAK (SEQ ID NO:59), or a fragment or variant thereof with at least 75%, 80%, 85%, 90%, 95%, 96%, 97%, 98%, or 99% sequence identity thereto. In some embodiments, the variant includes mutations at one or more of the bolded residues, optionally wherein one or more of the mutations is discussed in more detail below. Orthologous CH2 amino acid sequences useful for the disclosed antibodies are described in more detail in international PCT applications WO2017/011342 and WO2017/106462, each of which are incorporated herein incorporated by reference in its entirety.

The CH2 sequences can include or otherwise be linked an N-terminal hinge region peptide that connects the N-terminal variable domain-constant domain (e.g., CH1) to the CH2 domain. In addition, the hinge region typically provides both flexibility between the N-terminal variable domain-constant domain segment and CH2 domain, as well as amino acid sequence motifs that form disulfide bridges between heavy chains (e.g. the first and the third polypeptide chains).

In some embodiments, the CH3 sequences are from an IgG isotype, such as an IgG1 isotype. In some embodiments, the CH3 sequence is from an IgA isotype. In particular embodiments, the CH3 sequence is UniProt accession number P01857 (SEQ ID NO:26), amino acids 224-330: GQPREPQVYTLPPSRDELTKNQVSLTCLVKGFYPSDIAVEWESNGQPENNY KTTPPVLDSDGSFFLYSKLTVDKSRWQQGNVFSCSVMHEALHNHYTQKSLS LSPGK (SEQ ID NO:60), or a fragment or variant thereof with at least 75%, 80%, 85%, 90%, 95%, 96%, 97%, 98%, or 99% sequence identity thereto. In some embodiments, the variant includes mutations at one or more of the bolded residues, optionally wherein one or more of the mutations is discussed in more detail below.

In some embodiments, a CH3 sequence is a segment of an endogenous CH3 sequence, or an engineered or modified sequence.

One or more heavy chain mutations (e.g., insertions, deletion, and/or substitutions) can be incorporated into the sequence or fragment utilized in the disclosed constructs. Exemplary mutations include those provided below with reference to UniProt accession number PODOX5 (SEQ ID NO:25) the corresponding residue in P01857 (SEQ ID NO:26), and exemplary domains (SEQ ID NOS:62-64) in parentheses, and likewise be identified or applied to the corresponding residues in other IgG reference sequences. For example, in particular embodiments, a CH3 sequence has an endogenous CH3 sequence that lacks the N-terminal amino acids G343(224) and Q344(225). In particular embodiments, a CH3 sequence has an endogenous CH3 sequence that lacks the C-terminal amino acids P447(328), G448(329) and K449(330). In particular embodiments, a CH3 sequence has an endogenous CH3 sequence that lacks both the N-terminal amino acids G343(224) and Q344(225) and the C-terminal amino acids P447(328), G448(329), and K449(330).

In certain embodiments, the CH3 sequences are naturally occurring sequences that have one or more substitutions. In particular embodiments, the mutations are one or more orthogonal mutations that are introduced into an endogenous CH3 sequence to guide specific pairing of specific CH3 sequences, reduce immunogenicity, or a combination thereof. In some embodiments, the CH3 sequence includes knob-hole orthogonal mutations; isoallotype mutations, either a S356(237)C or a Y351(232)C mutation that forms an engineered disulfide bridge with a CH3 domain containing an orthogonal mutation, or a combination thereof. In some preferred embodiments, the knob-hole orthogonal mutations combined with isoallotype mutations are the following mutational changes: D358(239)E, L360(241)M, T368(249)S, L370(251)A, and Y409(290)V. CH3 sequences engineered to reduce immunogenicity of the chimeric antibody by replacing specific amino acids of one allotype with those of another allotype and referred to herein as isoallotype mutations, as described in more detail in Stickler et al. (Genes Immun. 2011 April; 12(3): 213-221), which is herein incorporated by reference for all that it teaches. In particular embodiments, specific amino acids of the G1 ml allotype are replaced. In a preferred embodiment, isoallotype mutations D358(239)E and L360(241)M are made in the CH3 sequence. In some particular examples, a human IgG1 CH3 amino acid sequence can have the following mutational changes: P345(226)V; Y351(232)C; and a tripeptide insertion, 447(328)P, 448(329)G, 449(330)K. In other embodiments, a human IgG1 CH3 sequence with the following mutational changes: T368(249)K; and a tripeptide insertion, 447(328)K, 448(329)S, 449(330)C. In other embodiments, a human IgG1 CH3 has a sequence with the following mutational changes: Y349(230)C and a tripeptide insertion, 447(328)P, 448(329)G, 449(330)K, or a human IgG1 CH3 sequence with a 449(330)C mutation incorporated into an otherwise endogenous CH3 sequence.

In some embodiments, the Fc region (e.g., CH2-CH3(—CH4) domain(s)) has been modifications to increase the Fc-mediated effector function. For example, in some embodiments, the Fc domain may contain one or more amino acid insertions, deletions or substitutions that enhance binding to specific Fc receptors that specifically expressed on tumors or tumor-associated neovasculature or are overexpressed on tumors or tumor-associated neovasculature relative to normal tissue.

The therapeutic outcome in patients treated with rituximab (a chimeric mouse/human IgG1 monoclonal antibody against CD20) for non-Hodgkin's lymphoma or Waldenstrom's macroglobulinemia correlated with the individual's expression of allelic variants of Fcγ receptors with distinct intrinsic affinities for the Fc domain of human IgG1. In particular, patients with high affinity alleles of the low affinity activating Fc receptor CD16A (FcγRIIIA) showed higher response rates and, in the cases of non-Hodgkin's lymphoma, improved progression-free survival. In another embodiment, the Fc domain may contain one or more amino acid insertions, deletions or substitutions that reduce binding to the low affinity inhibitory Fc receptor CD32B (FcγRIIB) and retain wild-type levels of binding to or enhance binding to the low affinity activating Fc receptor CD16A (FcγRIIIA). In a preferred embodiment, the Fc domain contains amino acid insertions, deletions or substitutions that enhance binding to CD16A. A large number of substitutions in the Fc domain of human IgG1 that increase binding to CD16A and reduce binding to CD32B are known in the art and are described in Stavenhagen, et al., Cancer Res., 57(18):8882-90 (2007). Exemplary variants of human IgG1 Fc domains with reduced binding to CD32B and/or increased binding to CD16A contain F245(130)L, R294(175)P, Y302(183)L, V307(188)I or P398(279)L substitutions. These amino acid substitutions may be present in a human IgG1 Fc domain in any combination. In one embodiment, the human IgG1 Fc domain variant contains a F245(130)L, R294(175)P and Y302(183)L substitution. In another embodiment, the human IgG1 Fc domain variant contains a F245L, R294P, Y302L, V307(188)I and P398(279)L substitution.

Another embodiment includes IgG₂₄ hybrids and IgG₂₄ mutants that have reduce binding to FcR which increase their half-life. Representative IG₂₄ hybrids and IgG4 mutants are described in Angal, S. et al. (1993) “A Single Amino Acid Substitution Abolishes The Heterogeneity Of Chimeric Mouse/Human (Igg4) Antibody,” Molec. Immunol. 30(1):105-108; Mueller, J. P. et al. (1997) “Humanized Porcine VCAM-Specific Monoclonal Antibodies With Chimeric Igg2/G4 Constant Regions Block Human Leukocyte Binding To Porcine Endothelial Cells,” Mol. Immun. 34(6):441-452; and U.S. Pat. No. 6,982,323. In some embodiments the IgG1 and/or IgG2 domain is deleted for example, Angal, s. et al. describe IgG1 and IgG2 having serine 241(122) replaced with a proline.

Substitutions, additions or deletions in the chimeric antibodies may be in the Fc region of the antibody and may thereby serve to modify the binding affinity of the antibody to one or more FcγR. Methods for modifying antibodies with modified binding to one or more FcγR are known in the art, see, e.g., PCT Publication Nos. WO 04/029207, WO 04/029092, WO 04/028564, WO 99/58572, WO 99/51642, WO 98/23289, WO 89/07142, WO 88/07089, and U.S. Pat. Nos. 5,843,597 and 5,642,821. In one particular embodiment, the modification of the Fc region results in an antibody with an altered antibody-mediated effector function, an altered binding to other Fc receptors (e.g., Fc activation receptors), an altered antibody-dependent cell-mediated cytotoxicity (ADCC) activity, an altered C1q binding activity, an altered complement-dependent cytotoxicity activity (CDC), a phagocytic activity, or any combination thereof.

In some embodiments, encompasses antibodies whose Fc region will have been modified so that the molecule will exhibit altered Fc receptor (FcR) binding activity, for example to exhibit decreased activity toward activating receptors such as FcγRIIA or FcγRIIIA, or increased activity toward inhibitory receptors such as FcγRIIB. Preferably, such antibodies will exhibit decreased antibody-dependent cell-mediated cytotoxicity (ADCC) or complement dependent cytotoxicity (CDC) activities (relative to a wild-type Fc receptor).

Modifications that affect Fc-mediated effector function are well known in the art (see U.S. Pat. No. 6,194,551, and WO 00/42072; Stavenhagen, J. B. et al. (2007) “Fc Optimization Of Therapeutic Antibodies Enhances Their Ability To Kill Tumor Cells In Vitro And Controls Tumor Expansion In Vivo Via Low-Affinity Activating Fcgamma Receptors,” Cancer Res. 57(18):8882-8890; Shields, R. L. et al. (2001) “High Resolution Mapping of the Binding Site on Human IgG1 for FcγRI, FcγRII, FcγRIII, and FcRn and Design of IgG1 Variants with Improved Binding to the FcγR,” J. Biol. Chem. 276(9):6591-6604). Exemplary variants of human IgG1 Fc domains with reduced binding to FcγRIIA or FcγRIIIA, but unchanged or enhanced binding to FcγRIIB, include S241(122)A, H270(151)A, S269(150)G, E271(152)A, E295(176)A, E295(176)D, Y298(179)F, R303(184)A,V305(186)A, A329(210)G, K324(205)A, E335(216)A, K336(217)A, K340(221)A, A341(222)A, D378(259)A.

See also U.S. Published Application Nos. 2021/0179734, 20110150867, 2005/0037000 and 2005/0064514 each of which is specifically incorporated by reference herein in its entirety.

Additional mutations include, but are not limited to, those of Table 8:

TABLE 8
Exemplary Fc Mutations

| | | Phenotype | | | |
|---|---|---|---|---|---|
| Fc Mutant | Fc Amino Acid Residue Changes | IIa | IIb | IIIa | Rn |
| GA | Gly238(119)Ala | ++++ | ++ | ++ | ++ |
| ALIE | Ala332(213)Leu/Ile334(215)Glu | ++ | ++ | ++++ | ++ |
| GAALIE | Gly238(119)Ala/Ala332(213)Leu/Ile334(215)Glu | ++++ | + | ++++ | ++ |
| VII | Chugai/Ravetch Cancer Cell paper | + | ++++ | − | ++ |
| GRLR | Gly238(119)Arg/Leu330(211)Arg | − | − | − | ++ |
| LS | Met430(311)Leu/Asn436(317)Ser | ++ | ++ | ++ | ++++ |
| GAALIE LS | Gly238(119)Ala/Ala332(213)Leu/ | | | | |
| | Ile334(215)Glu Met430(311)Leu/ | | | | |
| | Asn436(317)Ser | ++++ | + | ++++ | ++++ |
| LALA | Leu236(117)Ala/Leu237(118)Ala | | | | |
| LALA PG | Leu236(117)Ala/Leu237(118)Ala Pro331(212)Gly | | | | |
| GASDALIE | Gly238(119)Ala/Ser241(122)Asp/Ala332(213)Leu/Ile334(215)Glu | | | | |

See also, Lo, et al., “Effector attenuating substitutions that maintain antibody stability and reduce toxicity in mice.” J Biol Chem. (2017) 292:3900-8. doi: 10.1074/jbc.M116.767749, Oganesyan, et al., “Structural characterization of a human Fc fragment engineered for lack of effector functions.” Acta Crystallogr D Biol Crystallogr. (2008) 64:700-4. doi: 10.1107/S0907444908007877, and Chu, et al., “Inhibition of B cell receptor-mediated activation of primary human B cells by coengagement of CD19 and FcgammaRIIb with Fc-engineered antibodies.” Mol Immunol 45:3926-3933 (2008), which are specifically incorporated by reference herein in their entireties.

Exemplary sequences include, but are not limited to, DKTHTCPPCPAPELLGGPSVFLFPPKPKDTLMISRTPEVTCVVVDVSHEDP EVKFNWYVDGVEVHNAKTKPREEQYNSTYRVVSVLTVLHQDWLNGKEYKCK VSNKALPAPIEKTISKAKGQPREPQVYTLPPSREEMTKNQVSLTCLVKGFY PSDIAVEWESNGQPENNYKTTPPVLDSDGSFFLYSKLTVDKSRWQQGNVFS CSVMHEALHNHYTQKSLSLSPG ((SEQ ID NO:61) Human IgG1 Hinge-CH2-CH3 Fc Sequence), and fragments and variants thereof with at least 75%, 80%, 85%, 90%, 95%, 96%, 97%, 98%, or 99% sequence identity thereto; RTVAAPSVFIFPPSDEQLKSGTASVVCLLNNFYPREAKVQWKVDNALQSGN SQESVTEQDSKDSTYSLSSTLTLSKADYEKHKVYACEVTHQGLSSPVTKSF NRGEC ((SEQ ID NO:24) Human IgKappa CL Sequence), and fragments and variants thereof with at least 75%, 80%, 85%, 90%, 95%, 96%, 97%, 98%, or 99% sequence identity thereto; ASTKGPSVFPLAPSSKSTSGGTAALGCLVKDYFPEPVTVSWNSGALTSGVH TFPAVLQSSGLYSLSSVVTVPSSSLGTQTYICNVNHKPSNTKVDKKVEPKS CDKTHTCPPCPAPELLGGPSVFLFPPKPKDTLMISRTPEVTCVVVDVSHED PEVKFNWYVDGVEVHNAKTKPREEQYNSTYRVVSVLTVLHQDWLNGKEYKC KVSNKALPAPIEKTISKAKGQPREPQVYTLPPSREEMTKNQVSLTCLVKGF YPSDIAVEWESNGQPENNYKTTPPVLDSDGSFFLYSKLTVDKSRWQQGNVF SCSVMHEALHNHYTQKSLSLSPG ((SEQ ID NO:62) Human IgG1 CH1-Hinge-C_{H}2-C_{H}3 Sequence), and fragments and variants thereof with at least 75%, 80%, 85%, 90%, 95%, 96%, 97%, 98%, or 99% sequence identity thereto; ASTKGPSVFPLAPSSKSTSGGTAALGCLVKDYFPEPVTVSWNSGALTSGVH TFPAVLQSSGLYSLSSVVTVPSSSLGTQTYICNVNHKPSNTKVDKKVEPKS CDKTHTCPPCPAPEAAGGPSVFLFPPKPKDTLMISRTPEVTCVVVDVSHED PEVKFNWYVDGVEVHNAKTKPREEQYNSTYRVVSVLTVLHQDWLNGKEYKC KVSNKALGAPIEKTISKAKGQPREPQVYTLPPCREEMTKNQVSLWCLVKGF YPSDIAVEWESNGQPENNYKTTPPVLDSDGSFFLYSKLTVDKSRWQQGNVF SCSVMHEALHNHYTQKSLSLSPG ((SEQ ID NO:63) Human IgG1 CH1-Hinge-CH2-CH3 Sequence (with L117A/L118A/P212G “LALA PG” FcR1, RII, RIII silencing; S237C/T249W “Knob” mutations illustrated with italics/bold/single underlining)), and fragments and variants thereof with at least 75%, 80%, 85%, 90%, 95%, 96%, 97%, 98%, or 99% sequence identity thereto; and ASTKGPSVFPLAPSSKSTSGGTAALGCLVKDYFPEPVTVSWNSGALTSGVH TFPAVLQSSGLYSLSSVVTVPSSSLGTQTYICNVNHKPSNTKVDKKVEPKS CDKTHTCPPCPAPEAAGGPSVFLFPPKPKDTLMISRTPEVTCVVVDVSHED PEVKFNWYVDGVEVHNAKTKPREEQYNSTYRVVSVLTVLHQDWLNGKEYKC KVSNKALGAPIEKTISKAKGQPREPQVCTLPPSREEMTKNQVSLSCAVKGF YPSDIAVEWESNGQPENNYKTTPPVLDSDGSFFLVSKLTVDKSRWQQGNVF SCSVMHEALHNHYTQKSLSLSPG* ((SEQ ID NO:64) Human IgG1 C_{H}1-Hinge-C_{H}2-C_{H}3 Sequence (italics with L117A/L118A/P212G “LALA PG” FcR1, RII, RIII silencing; Y232C/T249S/L251A/Y290V “Hole” mutations illustrated with italics/bold/single underlining)), and fragments and variants thereof with at least 75%, 80%, 85%, 90%, 95%, 96%, 97%, 98%, or 99% sequence identity thereto.

### 4. Linkers and Signal Sequences

a. Signal Sequences

A signal peptide (sometimes referred to as signal sequence, targeting signal, localization signal, localization sequence, transit peptide, leader sequence or leader peptide) is a short peptide (usually 16-30 amino acids long) present at the N-terminus (or occasionally nonclassically at the C-terminus or internally) of most newly synthesized proteins that are destined toward the secretory pathway. These proteins include those that reside either inside certain organelles (the endoplasmic reticulum, Golgi or endosomes), secreted from the cell, or inserted into most cellular membranes. At the end of the signal peptide there is typically a stretch of amino acids that is recognized and cleaved by signal peptidase and therefore named cleavage site.

All of the disclosed fusion proteins are expressly disclosed with and without a signal sequence. An exemplary, non-limiting signal sequence used in the constructs of the examples below is MEWSWVFLFFLSVTTGVHS (SEQ ID NO:27).

b. Linkers

The term “linker” as used herein includes, without limitation, peptide linkers. The peptide linker can be any size provided it does not interfere with the binding of the epitope by the variable regions or VLRB antigen binding domain. In some embodiments, the linker includes one or more glycine and/or serine amino acid residues. The linker is chosen based on its intended purpose, which can include linking VLRB and/or ScFv antigen binding domains to the CL and/or C1 and/or CH3 and/or CH4 domains of the light and heavy chains, and/or to permit the heavy chain(s) and light chain(s) of an ScFv to bind together in their proper conformational orientation. Di-, tri-, and other multivalent scFvs typically include three or more linkers. The linkers can be the same, or different, in length and/or amino acid composition. Therefore, the number of linkers, composition of the linker(s), and length of the linker(s) can be determined based on the desired valency of the scFv as is known in the art. The linker(s) can allow for or drive formation of a di-, tri-, and other multivalent scFv. Exemplary flexible linkers include, but are not limited to, the amino acid sequences Gly-Ser, Gly-Ser-Gly-Ser (SEQ ID NO:28), Ala-Ser, Gly-Gly-Gly-Ser (SEQ ID NO:29), (Gly₄-Ser)₂ (SEQ ID NO:30) and (Gly₄-Ser)₄ (SEQ ID NO:31), (Gly-Gly-Gly-Gly-Ser)₃ (SEQ ID NO:32), and Gly4-Ala-Gly4 (SEQ ID NO:33). Other linkers include those of Table 9 (V. Mallajosyula et al., Sci. Immunol. 10.1126/sciimmunol.abg5669 (2021).

TABLE 9
Exemplary Linkers
Sequence

(G₃S)₃ (SEQ ID NO: 35)
(G₃S)₆ (SEQ ID NO: 36)
S₂G
(S₂G)₃ (SEQ ID NO: 37)
(SG₂P)₂SG₂ (SEQ ID NO: 38)
(S₂G)₃SPVG₂ (SEQ ID NO: 39)
(G₂S)₂SPV(STP₂TPSP)₂G₂S (SEQ ID NO: 40)
(G₂S)₂SPV(STP₂TPSP)₄G₂S (SEQ ID NO: 41)
G₂S(GSP)₃G₂S (SEQ ID NO: 42)
S₂G(EA₃K)₃S₂G (SEQ ID NO: 43)
S₂GEA₃KALEAEA₃KS₂G (SEQ ID NO: 44)
(GS₂P)₃G₃S (SEQ ID NO: 45)
GA₂PA₃PAKQEA₃PAPA₂KAEAPA₃PA₂KA (SEQ ID NO: 46)
S₂G(G₂PQ)₃S₂G (SEQ ID NO: 47)
G₂S₂PG₂S₂ (SEQ ID NO: 48)
G₂S₂PG₂S₂PG₂S₂PG₂S₂ (SEQ ID NO: 49)
KLSG₄SG₄SG₄SAEAWYNLGNAY₂KQGDYQKAIEY₂QKALELDPN₂L
QRSAGASGASG₄AS (SEQ ID NO: 50)

### 5. Cargo

Any of the disclosed fusion proteins or chimeric antibodies can be further modified to include one or more cargos. Typically the cargo is an active agent. Agents to be delivered include therapeutic, nutritional, diagnostic, and prophylactic compounds. Proteins, peptides, carbohydrates, polysaccharides, nucleic acid molecules, and organic molecules, as well as diagnostic agents, can be delivered.

Active agents include drugs and imaging agents. Therapeutic agents include antibiotics, antivirals, anti-parasites (helminths, protozoans), anti-cancer (referred to herein as “chemotherapeutics”, including cytotoxic drugs such as doxorubicin, cyclosporine, mitomycin C, cisplatin and carboplatin, BCNU, 5FU, methotrexate, adriamycin, camptothecin, epothilones A-F, and taxol), antibodies and bioactive fragments thereof (including humanized, single chain, and chimeric antibodies), antigen and vaccine formulations, peptide drugs, anti-inflammatories, nutraceuticals such as vitamins, and oligonucleotide drugs (including DNA, RNAs including mRNAs, antisense, siRNA, miRNA, anti-miRNA, piRNA, aptamers, ribozymes, external guide sequences for ribonuclease P, and triplex forming agents such as tcPNAs). In some embodiments, the active agent is a vector, plasmid, or other polynucleotide encoding an oligonucleotide such as those discussed above.

Exemplary drugs to be delivered include anti-angiogenic agents, antiproliferative and chemotherapeutic agents. Such compositions can be referred to as an antibody drug conjugate (ADC). By combining the targeting of the antibody with the therapeutic drug (e.g., cancer-killing ability of cytotoxic drugs), ADCs allow sensitive discrimination between healthy and diseased tissue.

Non-limiting examples of antineoplastic drugs that damage DNA or inhibit DNA repair include carboplatin, carmustine, chlorambucil, cisplatin, cyclophosphamide, dacarbazine, daunorubicin, doxorubicin, epirubicin, idarubicin, ifosfamide, lomustine, mechlorethamine, mitoxantrone, oxaliplatin, procarbazine, temozolomide, and valrubicin. In some embodiments, the antineoplastic drug is temozolomide, which is a DNA damaging alkylating agent commonly used against glioblastomas. In some embodiments, the antineoplastic drug is a PARP inhibitor, which inhibits a step in base excision repair of DNA damage. For example, the PARP inhibitor can be Olaparib (C₂₄H₂₃FN₄O₃).

In some embodiments, the antineoplastic drug is a histone deacetylase inhibitor, which suppresses DNA repair at the transcriptional level and disrupt chromatin structure. In some embodiments, the antineoplastic drug is a proteasome inhibitor, which suppresses DNA repair by disruption of ubiquitin metabolism in the cell. Ubiquitin is a signaling molecule that regulates DNA repair. In some embodiments, the antineoplastic drug is a kinase inhibitor, which suppresses DNA repair by altering DNA damage response signaling pathways.

Additional antineoplastic drugs include, but are not limited to, alkylating agents (such as temozolomide, cisplatin, carboplatin, oxaliplatin, mechlorethamine, cyclophosphamide, chlorambucil, dacarbazine, lomustine, carmustine, procarbazine, chlorambucil and ifosfamide), antimetabolites (such as fluorouracil, gemcitabine, methotrexate, cytosine arabinoside, fludarabine, and floxuridine), some antimitotics, and vinca alkaloids such as vincristine, vinblastine, vinorelbine, and vindesine), anthracyclines (including doxorubicin, daunorubicin, valrubicin, idarubicin, and epirubicin, as well as actinomycins such as actinomycin D), cytotoxic antibiotics (including mitomycin, plicamycin, and bleomycin), and topoisomerase inhibitors (including camptothecins such as irinotecan and topotecan and derivatives of epipodophyllotoxins such as amsacrine, etoposide, etoposide phosphate, and teniposide) and cytoskeletal targeting drugs such as paclitaxel.

Prophylactics can include compounds alleviating swelling, reducing radiation damage, and anti-inflammatories.

Representative classes of diagnostic materials include paramagnetic molecules, fluorescent compounds, magnetic molecules, and radionuclides. Exemplary materials include, but are not limited to, metal oxides, such as iron oxide, metallic particles, such as gold particles, etc. Biomarkers can also be conjugated to the surface for diagnostic applications.

For example, for imaging, radioactive materials such as Technetium99 (^{99m}Tc) or magnetic materials such as Fe₂O₃ could be used. Examples of other materials include gases or gas emitting compounds, which are radioopaque. The most common imaging agents for brain tumors include iron oxide and gadolinium. Diagnostic agents can be radioactive, magnetic, or x-ray or ultrasound-detectable. Other detectable labels include, for example, a radioisotope, a fluorophore (e.g., fluorescein isothiocyanate (FJTC), phycoerythrin (PE)), an enzyme (e.g., alkaline phosphatase, horseradish peroxidase), element particles (e.g., gold particles) or a contrast agent. These may be encapsulated within, dispersed within, or conjugated to the polymer.

For example, a fluorescent label can be chemically conjugated to a polymer of the nanocarrier to yield a fluorescently labeled polymer. In other embodiments the label is a contrast agent. A contrast agent refers to a substance used to enhance the contrast of structures or fluids within the body in medical imaging. Contrast agents are known in the art and include, but are not limited to agents that work based on X-ray attenuation and magnetic resonance signal enhancement. Suitable contrast agents include iodine and barium.

Active agents can be selected based on the type of treatment being employed. Exemplary active agents for treating cancer, infections, and injury.

Chimeric antibodies can be chemically linked to a polypeptide by a peptide bond or by a chemical or peptide linker molecule of the type well known in the art. Methods for attaching a drug or other small molecule pharmaceutical to an antibody fragment are well known and can include used of bifunctional chemical linkers such as N-succinimidyl (4-iodoacetyl)-aminobenzoate; sulfosuccinimidyl(4-iodoacetyl)-aminobenzoate; 4-succinimidyl-oxycarbonyl-.A-inverted.-(2-pyridyldithio) toluene; sulfosuccinimidyl-6-[.alpha.-methyl-.A-inverted.-(pyridyldithiol)-toluami-do]hexanoate; N-succinimidyl-3-(-2-pyridyldithio)-proprionate; succinimidyl-6-[3 (-(-2-pyridyldithio)-proprionamido]hexanoate; sulfosuccinimidyl-6-[3 (-(-2-pyridyldithio)-propionamido]hexanoate; 3-(2-pyridyldithio)-propionyl hydrazide, Ellman's reagent, dichlorotriazinic acid, S-(2-thiopyridyl)-L-cysteine, and the like. Further bifunctional linking molecules are discussed in, for example, U.S. Pat. Nos. 5,349,066, 5,618,528, 4,569,789, 4,952,394, and 5,137,877.

The linker can be cleavable or noncleavable. Highly stable linkers can reduce the amount of payload that falls off in circulation, thus improving the safety profile, and ensuring that more of the payload arrives at the target cell. Linkers can be based on chemical motifs including disulfides, hydrazones or peptides (cleavable), or thioethers (noncleavable) and control the distribution and delivery of the active agent to the target cell. Cleavable and noncleavable types of linkers have been proven to be safe in preclinical and clinical trials (see, e.g., Brentuximab vedotin which includes an enzyme-sensitive linker cleavable by cathepsin; and Trastuzumab emtansine, which includes a stable, non-cleavable linker). In particular embodiments, the linker is a peptide linker cleavable by Edman degredation (Bachor, et al., Molecular diversity, 17 (3): 605-11 (2013)).

### E. Antigen Targets

The disclosed chimeric antibodies can be monospecific for one, or di- or multispecific for two or more targets. In some embodiments, the chimeric antibodies bind to an antigens that are specific to tumor cells or tumor-associated neovasculature, or are upregulated in tumor cells or tumor-associated neovasculature compared to normal tissue. In some embodiments, the chimeric antibodies bind to antigens that are specific to immune tissue, e.g., involved in the regulation of B and/or T cell activation in response to infectious disease causing agents, cancer, etc. In some embodiments, the chimeric antibodies are bi- or multi-specific, and bind both an tumor target and an immune cell target.

Some embodiments, may facilitate recruitment and/or activation of T cells. For example, a bispecific chimeric antibody may interact with the T cell receptor (anti-CD3 antibody or scFv) and also a cancer cell recognized by the VLRB antibody that preferably recruits and activatesT cells to lyse the cancer cells.

### 1. Immune Cell Targets

The antigen can be checkpoint ligand or receptor expressed by immunes cells or tumor cells, e.g., CTLA4, PD-1, PD-L1, PD-L2, B7-H3 (e.g., MGA271), B7-H4, BTLA, HVEM, TIM3, GALS, LAGS, LAG3, VISTA, KIR, 2B4, CD160, CGEN-15049, CHK 1, CHK2, A2aR, and B-7 family ligands.

The antigen can be co-stimulatory ligand or receptor expressed by immune cells or tumor cells, e.g., a costimulatory molecule comprises one or more of a MHC class I molecule, BTLA, a Toll ligand receptor, OX40, CD27, CD28, ICAM-1, LFA-1 (CD11a/CD18), ICOS (CD278), and 4-1BB (CD137), CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGLi, CD100 (SEMA4D), CD69, SLAMF6 (NTB-A, Ly108), SLAM (SLAMFi, CD150, IPO-3), BLAME (SLAMF8), SELPLG (CD162), LTBR, LAT, GADS, SLP-76, PAG/Cbp, CD40, and CD19a.

Other immune cell antigens include, but are not limited to CD3, CD4, CD8, NKG2A, TLR, and IDO.

### 2. Tumor Targets

The antigen expressed by the tumor may be specific to the tumor, or may be expressed at a higher level on the tumor cells as compared to non-tumor cells. Antigenic markers such as serologically defined markers known as tumor associated antigens, which are either uniquely expressed by cancer cells or are present at markedly higher levels (e.g., elevated in a statistically significant manner) in subjects having a malignant condition relative to appropriate controls, are contemplated for use in certain embodiments.

Tumor-associated antigens may include, for example, cellular oncogene-encoded products or aberrantly expressed proto-oncogene-encoded products (e.g., products encoded by the neu, ras, trk, and kit genes), or mutated forms of growth factor receptor or receptor-like cell surface molecules (e.g., surface receptor encoded by the c-erb B gene). Other tumor-associated antigens include molecules that may be directly involved in transformation events, or molecules that may not be directly involved in oncogenic transformation events but are expressed by tumor cells (e.g., carcinoembryonic antigen, CA-125, melonoma associated antigens, etc.) (see, e.g., U.S. Pat. No. 6,699,475; Jager, et al., Int. J. Cancer, 106:817-20 (2003); Kennedy, et al., Int. Rev. Immunol., 22:141-72 (2003); Scanlan, et al. Cancer Immun., 4:1 (2004)).

Genes that encode cellular tumor associated antigens include cellular oncogenes and proto-oncogenes that are aberrantly expressed. In general, cellular oncogenes encode products that are directly relevant to the transformation of the cell, and because of this, these antigens are particularly preferred targets for immunotherapy. An example is the tumorigenic neu gene that encodes a cell surface molecule involved in oncogenic transformation. Other examples include the ras, kit, and trk genes. The products of proto-oncogenes (the normal genes which are mutated to form oncogenes) may be aberrantly expressed (e.g., overexpressed), and this aberrant expression can be related to cellular transformation. Thus, the product encoded by proto-oncogenes can be targeted. Some oncogenes encode growth factor receptor molecules or growth factor receptor-like molecules that are expressed on the tumor cell surface. An example is the cell surface receptor encoded by the c-erbB gene. Other tumor-associated antigens may or may not be directly involved in malignant transformation. These antigens, however, are expressed by certain tumor cells and may therefore provide effective targets. Some examples are carcinoembryonic antigen (CEA), CA 125 (associated with ovarian carcinoma), and melanoma specific antigens.

In ovarian and other carcinomas, for example, tumor associated antigens are detectable in samples of readily obtained biological fluids such as serum or mucosal secretions. One such marker is CA125, a carcinoma associated antigen that is also shed into the bloodstream, where it is detectable in serum (e.g., Bast, et al., N. Eng. J. Med., 309:883 (1983); Lloyd, et al., Int. J. Canc., 71:842 (1997). CA125 levels in serum and other biological fluids have been measured along with levels of other markers, for example, carcinoembryonic antigen (CEA), squamous cell carcinoma antigen (SCC), tissue polypeptide specific antigen (TPS), sialyl TN mucin (STN), and placental alkaline phosphatase (PLAP), in efforts to provide diagnostic and/or prognostic profiles of ovarian and other carcinomas (e.g., Sarandakou, et al., Acta Oncol., 36:755 (1997); Sarandakou, et al., Eur. J. Gynaecol. Oncol., 19:73 (1998); Meier, et al., Anticancer Res., 17(4B):2945 (1997); Kudoh, et al., Gynecol. Obstet. Invest., 47:52 (1999)). Elevated serum CA125 may also accompany neuroblastoma (e.g., Hirokawa, et al., Surg. Today, 28:349 (1998), while elevated CEA and SCC, among others, may accompany colorectal cancer (Gebauer, et al., Anticancer Res., 17(4B):2939 (1997)).

The tumor associated antigen, mesothelin, defined by reactivity with monoclonal antibody K-1, is present on a majority of squamous cell carcinomas including epithelial ovarian, cervical, and esophageal tumors, and on mesotheliomas (Chang, et al., Cancer Res., 52:181 (1992); Chang, et al., Int. J. Cancer, 50:373 (1992); Chang, et al., Int. J. Cancer, 51:548 (1992); Chang, et al., Proc. Natl. Acad. Sci. USA, 93:136 (1996); Chowdhury, et al., Proc. Natl. Acad. Sci. USA, 95:669 (1998)). Using MAb K-1, mesothelin is detectable only as a cell-associated tumor marker and has not been found in soluble form in serum from ovarian cancer patients, or in medium conditioned by OVCAR-3 cells (Chang, et al., Int. J. Cancer, 50:373 (1992)). Structurally related human mesothelin polypeptides, however, also include tumor-associated antigen polypeptides such as the distinct mesothelin related antigen (MRA) polypeptide, which is detectable as a naturally occurring soluble antigen in biological fluids from patients having malignancies (see WO 00/50900).

A tumor antigen may include a cell surface molecule. Tumor antigens of known structure and having a known or described function, include the following cell surface receptors: HER1 (GenBank Accession No. U48722), HER2 (Yoshino, et al., J. Immunol., 152:2393 (1994); Disis, et al., Canc. Res., 54:16 (1994); GenBank Acc. Nos. X03363 and M17730), HER3 (GenBank Acc. Nos. U29339 and M34309), HER4 (Plowman, et al., Nature, 366:473 (1993); GenBank Acc. Nos. L07868 and T64105), epidermal growth factor receptor (EGFR) (GenBank Acc. Nos. U48722, and KO3193), vascular endothelial cell growth factor (GenBank No. M32977), vascular endothelial cell growth factor receptor (GenBank Acc. Nos. AF022375, 1680143, U48801 and X62568), insulin-like growth factor-I (GenBank Acc. Nos. X00173, X56774, X56773, X06043, European Patent No. GB 2241703), insulin-like growth factor-II (GenBank Acc. Nos. X03562, X00910, M17863 and M17862), transferrin receptor (Trowbridge and Omary, Proc. Nat. Acad. USA, 78:3039 (1981); GenBank Acc. Nos. X01060 and M11507), estrogen receptor (GenBank Acc. Nos. M38651, X03635, X99101, U47678 and M12674), progesterone receptor (GenBank Acc. Nos. X51730, X69068 and M15716), follicle stimulating hormone receptor (FSH-R) (GenBank Acc. Nos. Z34260 and M65085), retinoic acid receptor (GenBank Acc. Nos. L12060, M60909, X77664, X57280, X07282 and X06538), MUC-1 (Barnes, et al., Proc. Nat. Acad. Sci. USA, 86:7159 (1989); GenBank Acc. Nos. M65132 and M64928) NY-ESO-1 (GenBank Acc. Nos. AJ003149 and U87459), NA 17-A (PCT Publication No. WO 96/40039), Melan-A/MART-1 (Kawakami, et al., Proc. Nat. Acad. Sci. USA, 91:3515 (1994); GenBank Acc. Nos. U06654 and U06452), tyrosinase (Topalian, et al., Proc. Nat. Acad. Sci. USA, 91:9461 (1994); GenBank Acc. No. M26729; Weber, et al., J. Clin. Invest, 102:1258 (1998)), Gp-100 (Kawakami, et al., Proc. Nat. Acad. Sci. USA, 91:3515 (1994); GenBank Acc. No. S73003, Adema, et al., J. Biol. Chem., 269:20126 (1994)), MAGE (van den Bruggen, et al., Science, 254:1643 (1991)); GenBank Acc. Nos. U93163, AF064589, U66083, D32077, D32076, D32075, U10694, U10693, U10691, U10690, U10689, U10688, U10687, U10686, U10685, L18877, U10340, U10339, L18920, U03735 and M77481), BAGE (GenBank Acc. No. U19180; U.S. Pat. Nos. 5,683,886 and 5,571,711), GAGE (GenBank Acc. Nos. AF055475, AF055474, AF055473, U19147, U19146, U19145, U19144, U19143 and U19142), any of the CTA class of receptors including in particular HOM-MEL-40 antigen encoded by the SSX2 gene (GenBank Acc. Nos. X86175, U90842, U90841 and X86174), carcinoembryonic antigen (CEA, Gold and Freedman, J. Exp. Med., 121:439 (1985); GenBank Acc. Nos. M59710, M59255 and M29540), and PyLT (GenBank Acc. Nos. J02289 and J02038); p97 (melanotransferrin) (Brown, et al., J. Immunol., 127:539-46 (1981); Rose, et al., Proc. Natl. Acad. Sci. USA, 83:1261-61 (1986)).

Additional tumor associated antigens include thymus leukemia antigen (TL), prostate surface antigen (PSA) (U.S. Pat. Nos. 6,677,157; 6,673,545); β-human chorionic gonadotropin β-HCG) (McManus, et al., Cancer Res., 36:3476-81 (1976); Yoshimura, et al., Cancer, 73:2745-52 (1994); Yamaguchi, et al., Br. J. Cancer, 60:382-84 (1989): Alfthan, et al., Cancer Res., 52:4628-33 (1992)); glycosyltransferase β-1,4-N-acetylgalactosaminyltransferases (GalNAc) (Hoon, et al., Int. J. Cancer, 43:857-62 (1989); Ando, et al., Int. J. Cancer, 40:12-17 (1987); Tsuchida, et al., J. Natl. Cancer, 78:45-54 (1987); Tsuchida, et al., J. Natl. Cancer, 78:55-60 (1987)); NUC18 (Lehmann, et al., Proc. Natl. Acad. Sci. USA, 86:9891-95 (1989); Lehmann, et al., Cancer Res., 47:841-45 (1987)); melanoma antigen gp75 (Vijayasardahi, et al., J. Exp. Med., 171:1375-80 (1990); GenBank Accession No. X51455); human cytokeratin 8; high molecular weight melanoma antigen (Natali, et al., Cancer, 59:55-63 (1987); keratin 19 (Datta, et al., J. Clin. Oncol., 12:475-82 (1994)).

Tumor antigens of interest include antigens regarded in the art as “cancer/testis” (CT) antigens that are immunogenic in subjects having a malignant condition (Scanlan, et al., Cancer Immun., 4:1 (2004)). CT antigens include at least 19 different families of antigens that contain one or more members and that are capable of inducing an immune response, including but not limited to MAGEA (CT1); BAGE (CT2); MAGEB (CT3); GAGE (CT4); SSX (CT5); NY-ESO-1 (CT6); MAGEC (CT7); SYCP1 (C8); SPANXB1 (CT11.2); NA88 (CT18); CTAGE (CT21); SPA17 (CT22); OY-TES-1 (CT23); CAGE (CT26); HOM-TES-85 (CT28); HCA661 (CT30); NY-SAR-35 (CT38); FATE (CT43); and TPTE (CT44).

Additional tumor antigens that can be targeted, including a tumor-associated or tumor-specific antigen, include, but not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAA0205, Mart2, Mum-1, 2, and 3, neo-PAP, myosin class I, OS-9, pml-RARa fusion protein, PTPRK, K-ras, N-ras, Triosephosphate isomeras, Bage-1, Gage 3,4,5,6,7, GnTV, Herv-K-mel, Lage-1, Mage-A1,2,3,4,6,10,12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, $-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD68KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding proteincyclophilin C-associated protein), TAAL6, TAG72, TLP, TPS, CD19 (expressed, e.g., in B cell acute leukemia), EpCAM (expressed, e.g., in CTCs), CD20 (expressed, e.g., in B cell acute leukemia), and CD45 (expressed, e.g., in CTCs). Other tumor-associated and tumor-specific antigens are known to those of skill in the art and are suitable for targeting by the disclosed fusion proteins.

The antigen may be specific to tumor neovasculature or may be expressed at a higher level in tumor neovasculature when compared to normal vasculature. Exemplary antigens that are over-expressed by tumor-associated neovasculature as compared to normal vasculature include, but are not limited to, VEGF/KDR, Tie2, vascular cell adhesion molecule (VCAM), endoglin and α₅β₃ integrin/vitronectin. Other antigens that are over-expressed by tumor-associated neovasculature as compared to normal vasculature are known to those of skill in the art and are suitable for targeting by the disclosed fusion proteins.

In another embodiment, the chimeric antibody specifically binds to a chemokine or a chemokine receptor. Chemokines are soluble, small molecular weight (8-14 kDa) proteins that bind to their cognate G-protein coupled receptors (GPCRs) to elicit a cellular response, usually directional migration or chemotaxis. Tumor cells secrete and respond to chemokines, which facilitate growth that is achieved by increased endothelial cell recruitment and angiogenesis, subversion of immunological surveillance and maneuvering of the tumoral leukocyte profile to skew it such that the chemokine release enables the tumor growth and metastasis to distant sites. Thus, chemokines are vital for tumor progression.

Based on the positioning of the conserved two N-terminal cysteine residues of the chemokines, they are classified into four groups namely CXC, CC, CX3C and C chemokines. The CXC chemokines can be further classified into ELR+ and ELR− chemokines based on the presence or absence of the motif ‘glu-leu-arg (ELR motif)’ preceding the CXC sequence. The CXC chemokines bind to and activate their cognate chemokine receptors on neutrophils, lymphocytes, endothelial and epithelial cells. The CC chemokines act on several subsets of dendritic cells, lymphocytes, macrophages, eosinophils, natural killer cells but do not stimulate neutrophils as they lack CC chemokine receptors except murine neutrophils. There are approximately 50 chemokines and only 20 chemokine receptors, thus there is considerable redundancy in this system of ligand/receptor interaction.

Chemokines elaborated from the tumor and the stromal cells bind to the chemokine receptors present on the tumor and the stromal cells. The autocrine loop of the tumor cells and the paracrine stimulatory loop between the tumor and the stromal cells facilitate the progression of the tumor. Notably, CXCR2, CXCR4, CCR2 and CCR7 play major roles in tumorigenesis and metastasis. CXCR2 plays a vital role in angiogenesis and CCR2 plays a role in the recruitment of macrophages into the tumor microenvironment. CCR7 is involved in metastasis of the tumor cells into the sentinel lymph nodes as the lymph nodes have the ligand for CCR7, CCL21. CXCR4 is mainly involved in the metastatic spread of a wide variety of tumors.

### III. Nucleic Acids

### A. Isolated Nucleic Acid Molecules Encoding Fusion Proteins

Nucleic acids, including isolated nucleic acids, encoding fusion proteins, and variants thereof are disclosed. As used herein, “isolated nucleic acid” refers to a nucleic acid that is separated from other nucleic acid molecules that are present in a mammalian genome, including nucleic acids that normally flank one or both sides of the nucleic acid in a mammalian genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule independent of other sequences (e.g., a chemically synthesized nucleic acid, or a cDNA or genomic DNA fragment produced by PCR or restriction endonuclease treatment), as well as recombinant DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a recombinant DNA molecule that is part of a hybrid or fusion nucleic acid. A nucleic acid existing among hundreds to millions of other nucleic acids within, for example, a cDNA library or a genomic library, or a gel slice containing a genomic DNA restriction digest, is not to be considered an isolated nucleic acid.

Nucleic acids can be in sense or antisense orientation, or can be complementary to a reference sequence encoding the disclosed fusion proteins.

Nucleic acids can be DNA, RNA (e.g., mRNA), or nucleic acid analogs. Nucleic acid analogs can be modified at the base moiety, sugar moiety, or phosphate backbone. Such modification can improve, for example, stability, hybridization, or solubility of the nucleic acid.

Modifications at the base moiety can include deoxyuridine for deoxythymidine, and 5-methyl-2′-deoxycytidine or 5-bromo-2′-deoxycytidine for deoxycytidine. Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, for example, Summerton and Weller (1997) Antisense Nucleic Acid Drug Dev. 7:187-195; and Hyrup et al. (1996) Bioorgan. Med. Chem. 4:5-23. In addition, the deoxyphosphate backbone can be replaced with, for example, a phosphorothioate or phosphorodithioate backbone, a phosphoroamidite, or an alkyl phosphotriester backbone.

### B. Vectors and Host Cells Expressing Fusion Proteins

Nucleic acids, such as those described above, can be inserted into vectors for expression in cells. As used herein, a “vector” is a replicon, such as a plasmid, phage, or cosmid, into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence.

Nucleic acids in vectors can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, bacteriophage, baculoviruses, tobacco mosaic virus, herpes viruses, cytomegalo virus, retroviruses, vaccinia viruses, adenoviruses, and adeno-associated viruses. Numerous vectors and expression systems are commercially available from such corporations as Novagen (Madison, WI), Clontech (Palo Alto, CA), Stratagene (La Jolla, CA), and Invitrogen Life Technologies (Carlsbad, CA).

An expression vector can include a tag sequence. Tag sequences, are typically expressed as a fusion with the encoded polypeptide. Such tags can be inserted anywhere within the polypeptide including at either the carboxyl or amino terminus. Examples of useful tags include, but are not limited to, green fluorescent protein (GFP), glutathione S-transferase (GST), polyhistidine, c-myc, hemagglutinin, Flag™ tag (Kodak, New Haven, CT), maltose E binding protein and protein A.

Vectors containing nucleic acids to be expressed can be transferred into host cells. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Prokaryotic cells can be transformed with nucleic acids by, for example, electroporation or calcium chloride mediated transformation. Nucleic acids can be transfected into mammalian cells by techniques including, for example, calcium phosphate co-precipitation, DEAE-dextran-mediated transfection, lipofection, electroporation, or microinjection. Host cells (e.g., a prokaryotic cell or a eukaryotic cell such as a CHO cell) can be used to, for example, produce the fusion proteins described herein.

### IV. Methods of Manufacture

### A. Methods for Producing Isolated Nucleic Acid Molecules Encoding Fusion Proteins

Isolated nucleic acid molecules encoding fusion proteins can be produced by standard techniques, including, without limitation, common molecular cloning and chemical nucleic acid synthesis techniques. For example, polymerase chain reaction (PCR) techniques can be used to obtain an isolated nucleic acid encoding a variant costimulatory polypeptide. PCR is a technique in which target nucleic acids are enzymatically amplified. Typically, sequence information from the ends of the region of interest or beyond can be employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize a complementary DNA (cDNA) strand. Ligase chain reaction, strand displacement amplification, self-sustained sequence replication or nucleic acid sequence-based amplification also can be used to obtain isolated nucleic acids. See, for example, Lewis (1992) Genetic Engineering News 12:1; Guatelli et al. (1990) Proc. Natl. Acad. Sci. USA 87:1874-1878; and Weiss (1991) Science 254:1292-1293.

Isolated nucleic acids can be chemically synthesized, either as a single nucleic acid molecule or as a series of oligonucleotides (e.g., using phosphoramidite technology for automated DNA synthesis in the 3′ to 5′ direction). For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase can be used to extend the oligonucleotides, resulting in a single, double-stranded nucleic acid molecule per oligonucleotide pair, which then can be ligated into a vector. Isolated nucleic acids can also obtained by mutagenesis. Fusion protein encoding nucleic acids can be mutated using standard techniques, including oligonucleotide-directed mutagenesis and/or site-directed mutagenesis through PCR. See, Short Protocols in Molecular Biology. Chapter 8, Green Publishing Associates and John Wiley & Sons, edited by Ausubel et al, 1992. Examples of amino acid positions that can be modified include those described herein.

### B. Methods for Making Fusion Proteins

Fusion proteins can be obtained by, for example, chemical synthesis or by recombinant production in a host cell. To recombinantly produce a fusion protein, a nucleic acid containing a nucleotide sequence encoding the polypeptide can be used to transform, transduce, or transfect a bacterial or eukaryotic host cell (e.g., an insect, yeast, or mammalian cell). In general, nucleic acid constructs include a regulatory sequence operably linked to a nucleotide sequence encoding a fusion protein. Regulatory sequences (also referred to herein as expression control sequences) typically do not encode a gene product, but instead affect the expression of the nucleic acid sequences to which they are operably linked.

Useful prokaryotic and eukaryotic systems for expressing and producing polypeptides are well know in the art include, for example, Escherichia coli strains such as BL-21, and cultured mammalian cells such as CHO cells.

In eukaryotic host cells, a number of viral-based expression systems can be utilized to express fusion proteins. Viral based expression systems are well known in the art and include, but are not limited to, baculoviral, SV40, retroviral, or vaccinia based viral vectors.

Mammalian cell lines that stably express variant costimulatory polypeptides can be produced using expression vectors with appropriate control elements and a selectable marker. For example, the eukaryotic expression vectors pCR3.1 (Invitrogen Life Technologies) and p91023(B) (see Wong et al. (1985) Science 228:810-815) are suitable for expression of variant costimulatory polypeptides in, for example, Chinese hamster ovary (CHO) cells, COS-1 cells, human embryonic kidney 293 cells, NIH3T3 cells, BHK21 cells, MDCK cells, and human vascular endothelial cells (HUVEC). Following introduction of an expression vector by electroporation, lipofection, calcium phosphate, or calcium chloride co-precipitation, DEAE dextran, or other suitable transfection method, stable cell lines can be selected (e.g., by antibiotic resistance to G418, kanamycin, or hygromycin). The transfected cells can be cultured such that the polypeptide of interest is expressed, and the polypeptide can be recovered from, for example, the cell culture supernatant or from lysed cells. Alternatively, a fusion protein can be produced by (a) ligating amplified sequences into a mammalian expression vector such as pcDNA3 (Invitrogen Life Technologies), and (b) transcribing and translating in vitro using wheat germ extract or rabbit reticulocyte lysate.

Fusion proteins can be isolated using, for example, chromatographic methods such as DEAE ion exchange, gel filtration, and hydroxylapatite chromatography. For example, a costimulatory polypeptide in a cell culture supernatant or a cytoplasmic extract can be isolated using a protein G column. In some embodiments, variant costimulatory polypeptides can be “engineered” to contain an amino acid sequence that allows the polypeptides to be captured onto an affinity matrix. For example, a tag such as c-myc, hemagglutinin, polyhistidine, or Flag™ (Kodak) can be used to aid polypeptide purification. Such tags can be inserted anywhere within the polypeptide, including at either the carboxyl or amino terminus. Other fusions that can be useful include enzymes that aid in the detection of the polypeptide, such as alkaline phosphatase. Immunoaffinity chromatography also can be used to purify costimulatory polypeptides.

Methods for introducing random mutations to produce variant polypeptides are known in the art. Random peptide display libraries can be used to screen for desired fusion protein variants. Techniques for creating and screening such random peptide display libraries are known in the art (Ladner et al., U.S. Pat. No. 5,223,409; Ladner et al., U.S. Pat. No. 4,946,778; Ladner et al., U.S. Pat. No. 5,403,484 and Ladner et al., U.S. Pat. No. 5,571,698) and random peptide display libraries and kits for screening such libraries are available commercially.

### C. Methods of Manufacturing VLRB-Ig Antibodies

The disclosed chimeric antibodies can be prepared using any suitable methods known in the art. For example, a recombinant chimeric antibody can be produced by transfecting a host cell with one or more vectors encoding the disclosed fusion proteins in the desired combination(s). Exemplary descriptions of recombinant means of antibody generation and production include Delves, Antibody Production: Essential Techniques (Wiley, 1997); Shephard, et al., Monoclonal Antibodies (Oxford University Press, 2000); Goding, Monoclonal Antibodies: Principles And Practice (Academic Press, 1993); Current Protocols In Immunology (John Wiley & Sons, most recent edition); U.S. Pat. No. 4,816,397 (Boss et al.), U.S. Pat. Nos. 6,331,415 and 4,816,567 (both to Cabilly et al.), U.K. patent GB 2,188,638 (Winter et al.), and U.K. patent GB 2,209,757. Goeddel et al., Gene Expression Technology Methods in Enzymology Vol. 185 Academic Press (1991), and Borreback, Antibody Engineering, W. H. Freeman (1992). Additional information concerning the generation, design and expression of recombinant antibodies can be found in Mayforth, Designing Antibodies, Academic Press, San Diego (1993).

Antibodies can also be made by commercial vendors, that specialize in recombinant production of custom designs.

### V. Formulations

Pharmaceutical compositions including VLRB-Ig are provided. Pharmaceutical compositions containing peptides or polypeptides may be for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration. The compositions may also be administered using bioerodible inserts and may be delivered directly to an appropriate lymphoid tissue (e.g., spleen, lymph node, or mucosal-associated lymphoid tissue) or directly to an organ or tumor. The compositions can be formulated in dosage forms appropriate for each route of administration. In some embodiments, the compositions are formulated for enteral administration.

The term “effective amount” or “therapeutically effective amount” means a dosage sufficient to treat, inhibit, or alleviate one or more symptoms of the disorder being treated or to otherwise provide a desired pharmacologic and/or physiologic effect. The precise dosage will vary according to a variety of factors such as subject-dependent variables (e.g., age, immune system health, etc.), the disease, and the treatment being effected.

In some embodiment, the VLRB-Ig is administered in a range of 0.1-20 mg/kg based on extrapolation from tumor modeling and bioavailability. A most preferred range is 5-20 mg of VLRB-Ig/kg. Generally, for intravenous injection or infusion, dosage may be lower than when administered by an alternative route.

### A. Formulations for Parenteral Administration

In a preferred embodiment, the disclosed compositions, including those containing peptides and polypeptides, are administered in an aqueous solution, by parenteral injection or infusion. The formulation may also be in the form of a suspension or emulsion. In general, pharmaceutical compositions are provided including effective amounts of a peptide or polypeptide, and optionally include pharmaceutically acceptable diluents, preservatives, solubilizers, emulsifiers, adjuvants and/or carriers. Such compositions include sterile water, buffered saline (e.g., Tris-HCl, acetate, phosphate), pH and ionic strength; and optionally, additives such as detergents and solubilizing agents (e.g., TWEEN® 20, TWEEN 80, Polysorbate 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, filtration through a bacteria retaining filter, by incorporating sterilizing agents into the compositions, by irradiating the compositions, or by heating the compositions.

### B. Formulations for Enteral Administration

VLRB-Ig can also be formulated for oral delivery. Oral solid dosage forms are known to those skilled in the art. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the present proteins and derivatives. See, e.g., Remington's Pharmaceutical Sciences, 21st Ed. (2005, Lippincott, Williams & Wilins, Baltimore, Md. 21201) pages 889-964. The compositions may be prepared in liquid form, or may be in dried powder (e.g., lyophilized) form. Liposomal or polymeric encapsulation may be used to formulate the compositions. See also Marshall, K. In: Modern Pharmaceutics Edited by G. S. Banker and C. T. Rhodes Chapter 10, 1979. In general, the formulation will include the active agent and inert ingredients which protect the VLRB-Ig in the stomach environment, and release of the biologically active material in the intestine.

Liquid dosage forms for oral administration, including pharmaceutically acceptable emulsions, solutions, suspensions, and syrups, may contain other components including inert diluents; adjuvants such as wetting agents, emulsifying and suspending agents; and sweetening, flavoring, and perfuming agents.

### C. Controlled Delivery Polymeric Matrices

Compositions containing one or more VLRB-Ig can be administered in controlled release formulations. Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel. The matrix can also be incorporated into or onto a medical device to modulate an immune response, to prevent infection in an immunocompromised patient (such as an elderly person in which a catheter has been inserted or a premature child) or to aid in healing, as in the case of a matrix used to facilitate healing of pressure sores, decubitis ulcers, etc. Either non-biodegradable or biodegradable matrices can be used for delivery of VLRB-Ig, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl. Polymer Sci., 35:755-774 (1988).

Controlled release oral formulations may be desirable. VLRB-Ig can be incorporated into an inert matrix which permits release by either diffusion or leaching mechanisms, e.g., films or gums. Slowly disintegrating matrices may also be incorporated into the formulation. Another form of a controlled release is one in which the drug is enclosed in a semipermeable membrane which allows water to enter and push drug out through a single small opening due to osmotic effects. For oral formulations, the location of release may be the stomach, the small intestine (the duodenum, the jejunem, or the ileum), or the large intestine. Preferably, the release will avoid the deleterious effects of the stomach environment, either by protection of the active agent (or derivative) or by release of the active agent beyond the stomach environment, such as in the intestine. To ensure full gastric resistance an enteric coating (i.e., impermeable to at least pH 5.0) is essential. These coatings may be used as mixed films or as capsules such as those available from Banner Pharmacaps.

The devices can be formulated for local release to treat the area of implantation or injection and typically deliver a dosage that is much less than the dosage for treatment of an entire body. The devices can also be formulated for systemic delivery. These can be implanted or injected subcutaneously.

### VI. Methods of Use

### A. Therapeutic and Diagnostic Methods

Typically, VLRB-Ig are designed to bind to one or more target cells, e.g., by containing one or more domains (e.g., VLRB, ScFv, VHH, VH/VL, R, and/or L domains) that bind to an antigen, receptor, ligand, or other moiety on the target cells, as discussed above. This may serve to increase, induce, or enhance ADCC and/or CDC and/or ADCP, increase delivery of cargo to more of more target cells, increase or enhance the proximity or communication between two different target cells, recruit an effector cell, for example a cytotoxic T cell, to form a complex with a target cell, for example a tumor cell, and/or induce aggregation of one or more different type of target. For embodiments in which two or more cells are targeted (e.g., to increase or enhance the proximity or communication between two different target cells, recruit an effector cell to a target cell, induce aggregation of one or more different type of target, etc.), typically bi- or multispecific antibodies that include one or more domains (e.g., VLRB, ScFv, VH/VL, R, and/or L domains) that target two or more different antigens, receptors, ligands, or other moieties on two or more different target cells are utilized.

Embodiments in which one or more different cell types may be targeted, such as increased delivery of cargo to one or more target cells and induced or enhanced ADCC and/or CDC, the chimeric antibodies can be mono-, bi-, or multispecific. Thus, the disclosed methods typically include administering a subject in need thereof an effective amount of a VLRB-Ig antibody to bind to one or more target cell types. Such methods are preferably effective to achieve a diagnostic (e.g., detect the location or amount of target cells in the subject) or therapeutic result (e.g., a reduction or prevention of one or more symptoms of a disease or disorder).

For example, the VLRB-Ig antibodies provided herein may be useful in vivo and ex vivo as immune response-stimulating therapeutics, e.g., to treat cancer or infections.

In some embodiments, the VLRB-Ig are designed to facilitate antibody-dependent cellular cytotoxicity (ADCC) and/or complement-dependent cytotoxicity (CDC) and/or antibody-dependent cellular phagocytosis (ADCP) induced by the VLRB-Ig.

Also referred to as antibody-dependent cell-mediated cytotoxicity, ADCC is a mechanism of cell-mediated immune defense whereby an effector cell of the immune system actively lyses a target cell, whose membrane-surface antigens have been bound by specific antibodies. It is one of the mechanisms through which antibodies, as part of the humoral immune response, can be harnessed to limit and contain infection or cancer.

ADCC is independent of the immune complement system that also lyses targets but does not require any other cell. ADCC requires an effector cell which classically is known to be natural killer (NK) cells that typically interact with immunoglobulin G (IgG) antibodies. However, macrophages, neutrophils and eosinophils can also mediate ADCC, such as eosinophils killing certain parasitic worms known as helminths via IgE antibodies.

Thus, in some embodiments, the disclosed chimeric antibodies may exhibit ADCC activity or improved ADCC activity compared to a control. ADCC activity refers to the ability of an antibody to elicit an antibody-dependent cellular cytotoxicity (ADCC) reaction. ADCC is a cell-mediated reaction in which antigen-nonspecific cytotoxic cells that express FcRs (e.g., natural killer (NK) cells, neutrophils, and macrophages) recognize antibodies bound to the surface of a target cell and subsequently cause lysis of (i.e., “kill”) the target cell. The primary mediator cells in ADCC are natural killer (NK) cells. NK cells express FcγRIII, with FcγRIIA being an activating receptor and FcγRIIIB an inhibiting receptor. Monocytes express FcγRI, FcγRII and FcγRIII.

CDC is an effector function of IgG and IgM antibodies. When they are bound to surface antigen on target cell (e.g. bacterial or viral infected cell), the classical complement pathway is triggered by bonding protein C1q to these antibodies, resulting in formation of a membrane attack complex (MAC) and target cell lysis. Complement system is efficiently activated by human IgG1, IgG3 and IgM antibodies, weakly by IgG2 antibodies and it is not activated by IgG4 antibodies. ADCC and CDC are two mechanisms of action by which therapeutic antibodies can achieve an antitumor effect.

Thus, additionally or alternatively, the disclosed chimeric antibodies may exhibit CDC activity, or improved CDC activity compared to a control. CDC activity refers to the reaction of one or more components of the complement system that recognizes bound antibody on a target cell and subsequently causes lysis of the target cell.

ADCP is a potent mechanism of elimination of antibody-coated foreign particles such microbes or tumor cells. Engagement of FcγRIIa and FcγRI expressed on macrophages triggers a signaling cascade leading to the engulfment of the IgG-opsonised particle. See, e.g., Tay, et al., Front Immunol., 10: 332, doi: 10.3389/fimmu.2019.00332 (2019).

Thus, additionally or alternatively, the disclosed chimeric antibodies may exhibit ADCP activity, or improved ADCP activity compared to a control. ADCP activity refers to the ability of an antibody to elicit an antibody-dependent cellular phagocytosis (ADCP) reaction.

Additionally or alternatively, the disclosed bi- or multispecific chimeric antibodies can be used bring two or more target cell types into close proximity. Examples of paired target cells include, but are not limited to an immune cell and a cancer cell, for example a cytotoxic T cell and a tumor cell target, two different immune cells, etc. In this way, antibody can be facilitate signaling between the two cells. Examples of interactions and/or signaling between cells include, but are not limited to,
increased immune response activation induce by antigen presenting cell (APC) target interacting with a T cell target;
increased immune response effector functions such ADCC and/or CDC and/or ADCP induced by NK cell or macrophage target against a tumor or infected cell target; and
recruitment of CD8 cytotoxic T cells to a tumor with an anti-CD8:tumor-specific VLRB.

Additionally or alternatively, the disclosed chimeric antibodies can deliver conjugated cargo to target cells as introduce above. Delivery can be e.g., cytotoxic agents or diagnostic agents to tumor cells, immune response inducing agents such as proinflammtory cytokines to immune cells, etc.

### B. Target Cells and Disease for Treatment

Thus, the antigen binding domain, ligand, or receptor that is presented by the VLRB-Ig antibody can be selected based on the intended use, and designed to target the desired cell type or types. In some embodiments, one or more of the target cell types is a cancer cell. Cancers for which cells can be targeted include, but are not limited to, carcinomas, gliomas, sarcomas, blood and lymphatic system (including leukemias, lymphomas such as Hodgkin's lymphomas and non-Hodgkin's lymphomas, solitary plasmacytoma, multiple myeloma), cancers of the genitourinary system (including prostate cancer, bladder cancer, renal cancer, urethral cancer, penile cancer, testicular cancer,), cancers of the nervous system (including mengiomas, gliomas, glioblastomas, ependymomas) cancers of the head and neck (including squamous cell carcinomas of the oral cavity, nasal cavity, nasopharyngeal cavity, oropharyngeal cavity, larynx, and paranasal sinuses), lung cancers (including small cell and non-small cell lung cancer), gynecologic cancers (including cervical cancer, endometrial cancer, vaginal cancer, vulvar cancer ovarian and fallopian tube cancer), gastrointestinal cancers (including gastric, small bowel, colorectal, liver, hepatobiliary, and pancreatic cancers), skin cancers (including melanoma, squamous cell carcinomas, and basal cell carcinomas), breast cancer (including ductal and lobular cancer), and pediatric cancers (including neuroblastoma, Ewing's sarcoma, Wilms tumor, medulloblastoma).

In some embodiments, one or more of the target cell types is an immune cell. Immune cells include, but are not limited to T lymphocytes, Natural Killer cells, dendritic cells, antigen presenting cells, B cells, and macrophages.

In some preferred embodiments, the chimeric antibody is bispecific or multispecific for a tumor cell type and an immune cell type.

In some embodiments, the chimeric antibody is a bispecific T cell engager (BiTE). BiTE are reviewed in Tian, et al. “Bispecific T cell engagers: an emerging therapy for management of hematologic malignancies.” J Hematol Oncol 14, 75 (2021), pages 1-18, doi.org/10.1186/s13045-021-01084-4, which is specifically incorporated by reference herein in its entirety. BiTE typically target an immune cell target, such as CD3, and a tumor antigen simultaneously. The BiTE can be used to induce an immune response against a cancer (e.g., a tumor) having the tumor antigen. In some embodiments, a VLRB domain targets a tumor antigen and another binding/targeting domain (e.g., ScFv, VHH, VH/VL, R, and/or L domains) targets an immune cell. In other embodiments, a VLRB domain targets an immune cell and another binding/targeting domain (e.g., ScFv, VHH, VH/VL, R, and/or L domains) target the tumor antigen. Exemplary BiTE constructs are discussed herein and exemplified experimentally in the Examples below.

### C. Combination Therapies

The disclosed chimeric antibody compositions may be administered in conjunction with prophylactic vaccines, or therapeutic vaccines, which can be used to initiate or enhance a subject's immune response to a pre-existing antigen, such as a tumor antigen in a subject with cancer. The combinations can be administered in the same or separate admixtures.

The desired outcome of a prophylactic, therapeutic or de-sensitized immune response may vary according to the disease, according to principles well known in the art. Similarly, immune responses against cancer, allergens or infectious agents may completely treat a disease, may alleviate symptoms, or may be one facet in an overall therapeutic intervention against a disease. For example, the stimulation of an immune response against a cancer may be coupled with surgical, chemotherapeutic, radiologic, hormonal and other immunologic approaches in order to affect treatment. Treatment that is administered in addition to a first therapeutic agent to eradicate tumors can be referred to as adjuvant therapy. Adjuvant treatment is given to augment the primary treatment, such as surgery or radiation, to decrease the chance that the cancer will recur. This additional treatment can result in an amplification of the primary response as evidenced by a more potent and/or prolonged response.

There are five main types of adjuvant therapy (note that some of these are also used as primary/monotherapy as well): 1.) Chemotherapy that uses drugs to kill cancer cells, either by preventing them from multiplying or by causing the cells to self-destruct., 2.) Hormone therapy to reduce hormone production and prevent the cancer from growing, 3.) Radiation therapy that uses high-powered rays to kill cancer cells, 4.) Immunotherapy that attempts to influence the body's own immune system to attack and eradicate any remaining cancer cells. Immunotherapy can either stimulate the body's own defenses (cancer vaccines) or supplement them (passive administration of antibodies or immune cells)) Targeted therapy that targets specific molecules present within cancer cells, leaving normal, healthy cells alone. For example, many cases of breast cancer are caused by tumors that produce too much of a protein called HER2. Trastuzumab (Herceptin) is used as adjuvant therapy that targets HER2 positive tumors.

Typically adjuvant treatments are co-administered or given in conjunction with primary treatments to induce multiple mechanisms and increase the chances of eradicating the tumor. Immunotherapy, and vaccines in particular, offer the unique advantages of inducing a sustained antitumor effect with exquisite specificity and with the ability to circumvent existing immune tolerance.

In some embodiments, the disclosed chimeric antibodies, are administered as a secondary therapeutic agent following administration of a first therapeutic agent such as a cancer therapeutic agent. In other embodiments, the disclosed chimeric antibodies are the primary therapeutic agent and administered prior to a secondary therapeutic agent. The timing of the secondary therapeutic agent administration can range from day 0 to day 14 after the primary treatment and can include single or multiple treatments. In certain embodiments, a VLRB-Ig antibody is administered 1, 2, 3, 4, 5, 6, 7, 8, 9, 10, 11, 12, 13, or 14 days after administration of the primary treatment, or before a secondary agent.

In some embodiments, the second agent is one of the cargos discussed above. For example, in some embodiments, the representative therapeutic agents include, but are not limited to chemotherapeutic agents and pro-apoptotic agents, such as amsacrine, bleomycin, busulfan, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epirubicin, etoposide, fludarabine, fluorouracil, gemcitabine, hydroxycarbamide, idarubicin, ifosfamide, irinotecan, leucovorin, liposomal doxorubicin, liposomal daunorubicin, lomustine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, tegafur-uracil, temozolomide, teniposide, thiotepa, tioguanine, topotecan, treosulfan, vinblastine, vincristine, vindesine, vinorelbine, or a combination thereof. Representative pro-apoptotic agents include, but are not limited to fludarabinetaurosporine, cycloheximide, actinomycin D, lactosylceramide, 15d-PGJ(2) and combinations thereof, and/or immune checkpoint inhibitors such as PD-1, CTLA4, and B7-H1 antagonists e.g., anti-PD-1, anti-B7-H1, and anti-CTLA4 antibodies, etc.

### D. Additional Methods

Additional therapeutic, diagnostic and research-based methods are also provided. For example, the chimeric antibodies can be used to detect a selected agent, to block the activity of a selected agent, to purify an agent, as an imaging tool, and as a therapeutic agent.

Provided herein are methods of detecting an agent in a sample, including the steps of contacting the sample with a chimeric antibodies, under conditions in which the composition can bind to the agent in the sample, and detecting the composition bound to the agent in the sample. The bound composition indicates the agent in the sample. Detection methods are well known in the art. For example, the chimeric antibody can be labeled with a detectable tag. The detection method can be used to note the presence or absence of an agent in the sample. The detection method, however, can be further combined with quantification methods. In vitro assay methods include colorometric assays such as ELISA that allow the quantification of the agent based on a comparison to a control sample or samples of known agent quantity which can be used to establish an amount relative to a standard. The methods can also include radiometric assays that allow for quantification based on emitted radiation and fluorescent assays or any means of visualization and quantification described above.

The sample can be any sample to be tested including any biologic sample. Samples can include fluid samples (like water, blood, urine, etc.), tissue samples, culture samples, cellular samples, etc.

The chimeric antibodies may also be used to block the activity of any agent to which it binds, comparable to a blocking antibody. Thus also disclosed are methods of blocking the activity of an agent, including contacting the agent with a chimeric antibody under conditions for the composition to bind the agent. The binding of the composition to the agent blocks the activity of the agent. The contacting step can be in vivo or in vitro. Thus, for example, to reduce contamination of a sample, a chimeric antibody that binds a toxin can be added to the sample and block the toxin activity.

The chimeric antibodies may also be used to promote the activity of an agent to which it binds, comparable to an agonistic antibody. Thus also disclosed are methods of promoting the activity of an agent, including contacting the agent with the composition under conditions for the composition to bind the agent. The binding of the composition to the agent promotes the activity of the agent.

The chimeric antibodies disclosed herein can be used to determine the function of a gene with unknown function. Thus, disclosed herein are methods of using the disclosed chimeric antibodies in protein knock-down assays. For example, the disclosed compositions can be expressed in the cytoplasm of a cell which includes a gene of unknown function. When the RNA transcript is being translated in the cytoplasm of the cell, the disclosed composition can bind the protein product of the gene question. By monitoring the effect of the loss of protein expression has on the cell, the protein's function can be determined. Thus, specifically disclosed are chimeric antibodies specific for a gene product of unknown function. Also provided are methods of determining the function of a gene including introducing a chimeric antibody specific for the protein product of the gene into the cytoplasm of a cell expressing the gene and monitoring the effect due to the loss of protein product of the gene with unknown function.

The chimeric antibodies can also be used in imaging methods. For example, an imaging method can include administering to a subject an effective amount of a disclosed composition and detecting the localization of the bound composition in the subject. Examples of imaging methods are described above.

Methods of purification are provided. Disclosed herein are methods of purifying an agent from a sample can include contacting the sample with a chimeric antibodies under conditions for the composition to bind the agent and form a composition/agent complex; and isolating the agent from the composition/agent complex. For example, the composition can be bound to a column and the sample can be passed through the column under conditions that allow the agent in the sample to bind to the bound composition. The agent can subsequently be eluted from the column in a desired eluant. The purification methods would be useful as research methods and as commercial methods. For example, such a method would be useful in removing contaminants from pharmacological compounds.

### VII. Exemplary Embodiments

Non-limiting exemplary embodiments are illustrated in the FIGS. 2-6. FIG. 2 is an illustration of an engineered VLRB:CL-VLRB:CH1 tetravalent monospecific IgG chimera. The tetravalent monospecific MM3 VLRB human IgG1, kappa structure is being produced by recombinant expression in high yield CHO cell line, by replacing the VL and VH sequences of a human IgG1 VHCH gene and human VLCkappa gene with the MM3 VLRB sequence.

FIG. 3A is an illustration of an engineered tetravalent VLRB IgG with monovalent anti-CD3 scFv attached to the C-terminus of one of the IgG H chains (“knob” H chain) using knobs-in-hole method to insure correct H chain pairing. FIG. 3B is an illustration of an engineered trivalent VLRB IgG with monovalent anti-CD3 scFv attached to the N-terminus of one of the IgG H chains (“knob” H chain) using knobs-in-hole method to insure correct H chain pairing. FIGS. 3C and 3D illustrate two ways to construct a VLRB IgG with monovalent anti-CD3 scFv attached to the C-terminus (3C) or N-terminus (3D) of one of the IgG light chains a reverse of CL and CH1 on one of the H/L chain pairs (“crossmab”) and “knobs-in-hole” to insure correct H chain pairing.

FIG. 4 is an illustration of an engineered bivalent, bispecific VLRB-IgG chimera composed of an anti-CD3 monoclonal antibody with VLRB fused to the C-terminus of each CH3 domain.

FIG. 5 is an illustration of bivalent anti-CD3, bivalent VLRB antibody having 2× anti-CD3 mAb VL and VH, and 2×VLRB fused to the C-terminus of the CL domains, where the heavy chains further include CH1, CH2, and CH3 domains.

FIG. 6 is an illustration of VLRB-scFV chimera, wherein the VLRB domains can be 4× mono-, 2× bi-, 1× mono- & 1× tri-, or 1×-tetra specific, and the scFv can be the same (i.e., bivalent, monospecific), or different (i.e., monovalent, bispecific).

In an alternative exemplary embodiment, the anti-CD3 or anti-CD3 scFv domains of the foregoing figures are substituted with an anti-CD8 antibody (or scFv) or CD8 ligand domain. An exemplary CD8 ligand is thymus leukemia antigen. These antibodies are designed to recruit cytotoxic T cells all of which express CD8 and CD3, but not to recruit regulatory or other inhibitory T cells which will also express CD3 but not CD8, and preferably avoid recruitment of CD4 T cells. See, e.g., Clement, et al., J Immunol., 187(2): 654-663. doi:10.4049/jimmunol.1003941 (2011), Tsujimura, et al., International Immunology, Vol. 15, No. 11, pp. 1319-1326 doi: 10.1093/intimm/dxgl31 (2003), and WO 2014/164553, each of which is specifically incorporated by reference herein in its entirety.

In these embodiments, MM3 VLRB binds to plasmacytoma tumor cells and may recruit and activate T cells (via binding to CD3 or CD8) to lyse the tumor cells.

Exemplary non-limiting fusions proteins, include those utilized in the Examples below:

### (1) VLRB Human IgG1 Fc Fusion Proteins

(a) Bivalent MM3 VLRB₂ human IgG1 Fc

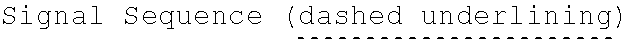

MM3 VLRB Sequence (bold)
Linker Sequence (lowercase)
Human IgG1 Hinge-C_{H}2-C_{H}3 Fc Sequence (italics)
(SEQ ID NO: 51)

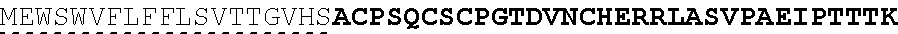

ILRLYINQITKLEPGVFDRLTQLTQLGLWDNQLQALPEGVEDRLVNLQKLY
LNQNQLLALPVGVFDKLTQLTYLDLNNNQLKSIPRGAFDNLKSLTHIWLYG
NPWDCECSDILYLKNWIVQHASIVNPHPYGGVDNVKCSGTNTPVRAVTEAS
TSPSKCPGYVATTTggggaggggDKTHTCPPCPAPELLGGPSVFLFPPKPK
DTLMISRTPEVTCVVVDVSHEDPEVKENWYVDGVEVHNAKTKPREEQYNST
YRVVSVLIVLHQDWLNGKEYKCKVSNKALPAPIEKTISKAKGQPREPQVYT
LPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPG*
*C-terminal Lysine removed per Cai, et al. (2011)
Biotechnol Bioeng 108: 404-412 doi:
10.1002/bit.22933.

Other VLRB human IgG1 Fe fusion proteins were produced by replacing the MM3 VLRB sequence with the sequence for the new VLRB and maintaining all other sequences of the above construct as shown below for the O13 VLRB₂ human IgG1 Fc fusion protein.

(b) Bivalent O13 VLRB₂ human IgG1 Fc

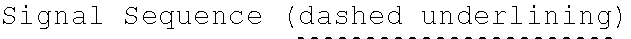

O13 VLRB Sequence (bold)
Linker Sequence (lowercase)
Human IgG1 Hinge-C_{H}2-C_{H}3 Fc Sequence (italics)
(SEQ ID NO: 52)

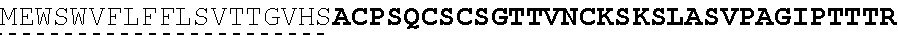

VLYLNDNQITKLEPGVFDRLVNLQTLWLNNNQLTSLPAGLEDSLTQLTILA
LDSNQLQALPVGVFGRLVDLQQLYLGSNQLSALPSAVEDRLVHLKELLMCC
NKLTELPRGIERLTHLTHLALDQNQLKSIPHGAFDRLSSLTHAYLEGNPWD
CECRDIMYLRNWVADHTSIVMRWDGKAVNDPDSAKCAGTNTPVRAVTEAST
SPSKCPGYVATTTggggaggggDKTHTCPPCPAPELLGGPSVFLFPPKPKD
TLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPG*
*C-terminal Lysine removed per Cai, et al. (2011)
Biotechnol Bioeng 108: 404-412 doi:
10.1002/bit.22933.

(2) Tetravalent MM3 VLRB4 human IgG1

(a) MM3 VLRB human IgG1 H Chain construct

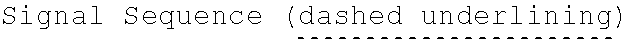

MM3 VLRB Sequence (bold)
Linker Sequence (lowercase)
Human IgG1 C_{H}1-Hinge-C_{H}2-C_{H}3 Sequence (italics)
(SEQ ID NO: 53)

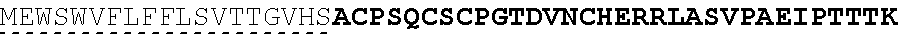

ILRLYINQITKLEPGVFDRLTQLTQLGLWDNQLQALPEGVEDRLVNLQKLY
LNQNQLLALPVGVFDKLTQLTYLDLNNNQLKSIPRGAFDNLKSLTHIWLYG
NPWDCECSDILYLKNWIVQHASIVNPHPYGGVDNVKCSGTNTPVRAVTEAS
TSPSKCPGYVATTTggggaggggASTKGPSVFPLAPSSKSTSGGTAALGCL
VKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKP
KDTLMISRTPEVTCVVVDVSHEDPEVKENWYVDGVEVHNAKTKPREEQYNS
TYRVVSVLIVLHQDWLNGKEYKCKVSNKALPAPIEKTISKAKGQPREPQVY
TLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPG*
(b) MM3 VLRB human IgKappa L Chain construct

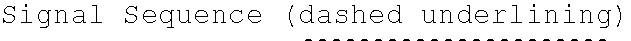

MM3 VLRB Sequence (bold)
Linker Sequence (lowercase)
Human IgKappa C_{L} Sequence (italics)
(SEQ ID NO: 54)

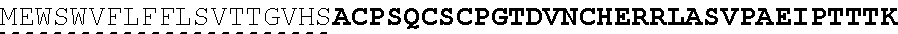

ILRLYINQITKLEPGVEDRLTQLTQLGLWDNQLQALPEGVEDRLVNLQKLY
LNQNQLLALPVGVFDKLTQLTYLDLNNNQLKSIPRGAFDNLKSLTHIWLYG
NPWDCECSDILYLKNWIVQHASIVNPHPYGGVDNVKCSGTNTPVRAVTEAS
TSPSKCPGYVATTTggggaggggRTVAAPSVFIFPPSDEQLKSGTASVVCL
LNNFYPREAKVQWKVDNALQSGNSQESVTEQDSKDSTYSLSSTLTLSKADY
EKHKVYACEVTHQGLSSPVTKSENRGEC

(3) MM3 VLRB4:TR66 anti-CD3 scFv human IgG1 BiTe

Utilizes “Knob-in-Hole” technology to facilitate correct H chain pairing and LALA PG mutations to silence FcR1, II and III binding while retaining FcRn binding to impart the dersiable blood T_{1/2} of IgG1 antibody.

All constructs are designed to increase/enhance tumor binding by incorporating multivalent MM3 VLRB presentation and to avoid crosslinking CD3 which would lead to T cell activation in the absence of tumor engagement, i.e., nonspecific T cell activation and killing, by incorporating monovalent anti-CD3 scFv presentation.

(a) MM3 VLRB human IgG1 H Chain S354C/T366W
“Knob” with C-terminal TR66 anti-CD3 scFv construct with
L234A/L235A/P329G FcR1, II, III silencing changes

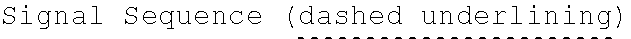

MM3 VLRB Sequence (bold)
Linker Sequence (lowercase)
Human IgG1 C_{H}1-Hinge-C_{H}2-C_{H}3 Sequence (italics with
L344A/L345A/P439G (also referred to elsewhere as
“L234A/L235A/P329G”) “LALA PG” FcR1, RII, RIII silencing;
S464C/T476W (also referred elsewhere was “S354C/
T366W”) “Knob” mutations illustrated with
italics/bold/single underlining)
TR66 anti-CD3 scFv Sequence (double underlining)
(SEQ ID NO: 55)

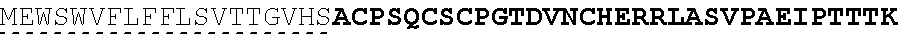

ILRLYINQITKLEPGVEDRLTQLTQLGLWDNQLQALPEGVFDRLVNLQKLY
LNQNQLLALPVGVFDKLTQLTYLDLNNNQLKSIPRGAFDNLKSLTHIWLYG
NPWDCECSDILYLKNWIVQHASIVNPHPYGGVDNVKCSGTNTPVRAVTEAS
TSPSKCPGYVATTTggggaggggASTKGPSVFPLAPSSKSTSGGTAALGCL
VKDYFPEPVTVSWNSGALTSGVHTFPAVLQSSGLYSLSSVVTVPSSSLGTQ
TYICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYNS
TYRVVSVLTVLHQDWLNGKEYKCKVSNKALGAPIEKTISKAKGQPREPQVY
TLPPCREEMTKNQVSLWCLVKGFYPSDIAVEWESNGQPENNYKTTPPVLDS
DGSFFLYSKLTVDKSRWQQGNVFSCSVMHEALHNHYTQKSLSLSPGgggga
ggggDVQLVQSGAEVKKPGASVKVSCKASGYTFTRYTMHWVRQAPGQGLEW
IGYINPSRGYTNYADSVKGRFTITTDKSTSTAYMELSSLRSEDTATYYCAR
YYDDHYCLDYWGQGTTVTVSSGEGTSTGSGGSGGSGGADDIVLTQSPATLS
LSPGERATLSCRASQSVSYMNWYQQKPGKAPKRWIYDTSKVASGVPARFSG
SGSGTDYSLTINSLEAEDAATYYCQQWSSNPLTFGGGTKVEIKS
(b) MM3 VLRB human IgG1 H Chain
Y349C/T366S/L368A/Y407V “Hole” construct with
L234A/L235A/P329G FcR1, II, III silencing changes

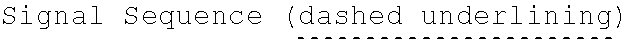

MM3 VLRB Sequence (bold)
Linker Sequence (lowercase)
Human IgG1 C_{H}1-Hinge-C_{H}2-C_{H}3 Sequence (italics with
L344A/L345A/P439G (also referred to elsewhere as
“L234A/L235A/P329G”) “LALA PG” FcR1, RII, RIII silencing;
Y459C/T476S/L478A/Y517V (also referred to elsewhere as
“Y349C/T366S/L368A/Y407V”) “Hole” mutations illustrated with
italics/bold/single underlining)
(SEQ ID NO: 56)

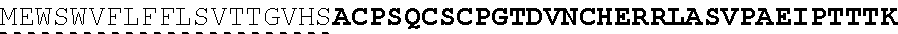

ILRLYINQITKLEPGVFDRLTQLTQLGLWDNQLQALPEGVEDRLVNLQKLY
LNQNQLLALPVGVFDKLTQLTYLDLNNNQLKSIPRGAFDNLKSLTHIWLYG
NPWDCECSDILYLKNWIVQHASIVNPHPYGGVDNVKCSGTNTPVRAVTEAS
TSPSKCPGYVATTTggggaggggASTKGPSVFPLAPSSKSTSGGTAALGCL
VKDYFPEPVTVSWNSGALTSGVHTFPAVLQSSGLYSLSSVVTVPSSSLGTQ
TYICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYNS
TYRVVSVLIVLHQDWLNGKEYKCKVSNKALGAPIEKTISKAKGQPREPQVC
TLPPSREEMTKNQVSLSCAVKGFYPSDIAVEWESNGQPENNYKTTPPVLDS
DGSFFLVSKLTVDKSRWQQGNVFSCSVMHEALHNHYTQKSLSLSPG*

(4) MM3 VLRB4:High Affinity Anti-CD3 scFv Human IgG1 BiTe

(a) MM3 VLRB Knob H chain with high affinity anti-
CD3 scFv at C-terminus

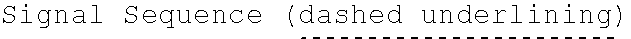

MM3 VLRB Sequence (bold)
Linker Sequence (lowercase)
Human IgG1 C_{H}1-Hinge-C_{H}2-C_{H}3 Sequence (italics with “knob”
mutations illustrated with italics/bold/single underlining)
High affinityh anti-CD3 scFv Sequence (double underlining)
(SEQ ID NO: 65)

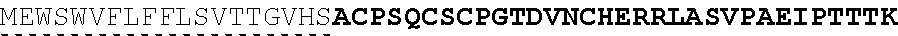

ILRLYINQITKLEPGVFDRLTQLTQLGLWDNQLQALPEGVEDRLVNLQKLY
LNQNQLLALPVGVFDKLTQLTYLDLNNNQLKSIPRGAFDNLKSLTHIWLYG
NPWDCECSDILYLKNWIVQHASIVNPHPYGGVDNVKCSGTNTPVRAVTEAS
TSPSKCPGYVATTTggggaggggASTKGPSVFPLAPSSKSTSGGTAALGCL
VKDYFPEPVTVSWNSGALTSGVHTFPAVLQSSGLYSLSSVVTVPSSSLGTQ
TYICNVNHKPSNTKVDKKVEPKSCDKTHTCPPCPAPEAAGGPSVFLFPPKP
KDTLMISRTPEVTCVVVDVSHEDPEVKFNWYVDGVEVHNAKTKPREEQYNS
TYRVVSVLIVLHQDWLNGKEYKCKVSNKALGAPIEKTISKAKGQPREPQVY
TLPPCREEMTKNQVSLWCLVKGFYPSDIAVEWESNGQPENNYKTTPPVLDS
DGSFFLYSKLTVDKSRWQQGNVFSCSVMHEALHNHYTQKSLSLSPGgggga
ggggEVOLVESGGGLVOPGGSLRLSCAASGFTFSTYAMNWVROAPGKGLEW
VGRIRSKYNNYATYYADSVKGRFTISRDDSKNTLYLOMNSLRAEDTAVYYC
VRHGNFGDSYVSWFAYWGQGTLVTVSSGKPGSGKPGSGKPGSGKPGSQAVV
TQEPSLTVSPGGTVTLTCGSSTGAVTTSNYANWVQQKPGKSPRGLIGGINK
RAPGVPARFSGSLLGGKAALTISGAQPEDEADYYCALWYSNHWVEGGGTKL
TVL.
(b)Knob H chain with high affinity anti-CD3 scFv
attached at N-terminus

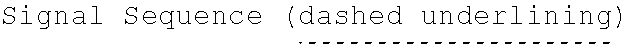

High affinity anti-CD3 scFv Sequence (double underlining)
Linker Sequence (lowercase)
Human IgG1 C_{H}1-Hinge-C_{H}2-C_{H}3 Sequence (italics with “knob”
mutations illustrated with italics/bold/single underlining)
(SEQ ID NO: 66)

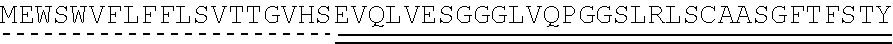

AMNWVRQAPGKGLEWVGRIRSKYNNYATYYADSVKGRFTISRDDSKNTLYL
QMNSLRAEDTAVYYCVRHGNFGDSYVSWFAYWGQGTLVTVSSGKPGSGKPG
SGKPGSGKPGSQAVVTQEPSLTVSPGGTVTLTCGSSTGAVTTSNYANWVQQ
KPGKSPRGLIGGTNKRAPGVPARESGSLLGGKAALTISGAQPEDEADYYCA
LWYSNHWVFGGGTKLTVLggggaggggASTKGPSVFPLAPSSKSTSGGTAA
LGCLVKDYFPEPVTVSWNSGALTSGVHTFPAVLQSSGLYSLSSVVTVPSSS
LGTQTYICNVNHKPSNTKVDKKVEPKSCDKTHTCPPCPAPEAAGGPSVFLF
PPKPKDTLMISRTPEVTCVVVDVSHEDPEVKFNWYVDGVEVHNAKTKPREE
QYNSTYRVVSVLIVLHQDWLNGKEYKCKVSNKALGAPIEKTISKAKGQPRE
PQVYTLPPCREEMTKNQVSLWCLVKGFYPSDIAVEWESNGQPENNYKTTPP
VLDSDGSFFLYSKLTVDKSRWQQGNVFSCSVMHEALHNHYTQKSLSLSPG.

In some embodiments, SEQ ID NO:53 forms a tetrameric antibody with itself, and/or one or more of MM3 VLRB L chains (e.g., SEQ ID NO:54).

In some embodiments, SEQ ID NO:55 forms a tetrameric antibody with MM3 VLRB hole H chain (e.g., SEQ ID NO:56), and/or one or more of MM3 VLRB L chains (e.g., SEQ ID NO:54).

In some embodiments, SEQ ID NO:65 forms a tetrameric antibody with MM3 VLRB hole H chain (e.g., SEQ ID NO:56), and/or one or more of MM3 VLRB L chains (e.g., SEQ ID NO:54).

In some embodiments, SEQ ID NO:66 forms a tetrameric antibody with MM3 VLRB hole H chain (e.g., SEQ ID NO:56), and/or one or more of MM3 VLRB L chains (e.g., SEQ ID NO:54).

The disclosed invention can be further understood by the following numbered paragraphs:

1. A heavy chain fusion protein comprising one or more variable lymphocyte receptor B (VLRB) antigen binding domains and a CH1 immunoglobulin domain (CH1) or a CL immunoglobulin domain (CL), optionally wherein the VLRB is at the N-terminus of fusion protein, the C-terminus of the fusion protein, or a combination thereof.

2. The heavy chain fusion protein of paragraph 1 further comprising an immunoglobulin hinge domain (hinge).

3. The heavy chain fusion protein of paragraphs 1 or 2 further comprising a CH2 immunoglobulin domain (CH2).

4. The heavy chain fusion protein of any one of paragraphs 1-3 further comprising a CH3 immunoglobulin domain (CH3).

5. The heavy chain fusion of any one of paragraphs 1-4 further comprising a CH4 immunoglobulin domain (CH4).

6. The heavy chain fusion protein of any one of paragraphs 1-5 further comprising a second (VLRB) antigen binding domain.

7. The heavy chain fusion protein of any one of paragraphs 1-6 further comprising a variable region of immunoglobulin heavy chain (VH), optionally wherein the VH is at the N-terminus of the fusion protein and the VLRB antigen binding domain and VH domain are fused to different ends of the fusion protein.

8. The heavy chain fusion protein of any one of paragraphs 1-7 further comprising one or more of a mono- or multivalent single-chain variable fragment (ScFv), VHH, a polypeptide ligand (L), or a polypeptide receptor (R), optionally at the N-terminus or C-terminus of the fusion protein and the VLRB antigen binding domain and mono- or multivalent single-chain variable fragment (ScFv), VHH, a polypeptide ligand (L), or a polypeptide receptor (R) are at different ends of the fusion protein.

9. The heavy chain fusion protein of any one of paragraphs 1-9 comprising a domain structure of Table 1.

10. A heavy chain fusion protein comprising a VLRB antigen binding domain and a structure of Table 1.

11. The heavy chain fusion protein of any one of paragraphs 1-10, wherein one or both of the VLRB antigen binding domains bind to a cancer or tumor antigen or an antigen expressed by immune cells.

12. The heavy chain fusion protein of any one of paragraphs 8-11, wherein one or more of the VH, ScFv, VHH, L, or R bind to a cancer or tumor antigen or an antigen expressed on by immune cells.

13. The heavy chain fusion protein of any one of paragraphs 1-12, wherein each of the immunoglobin domains are independently selected from a mammalian, optionally human, IgA, IgD, IgE, IgG, and IgM, or a variant thereof with at 70% sequence identity thereto.

14. The heavy chain fusion protein of paragraph 13, wherein the IgG is IgG1, IgG2, IgG3, and/or IgG4 and/or the IgA is IgA1 and/or IgA2.

15. The heavy chain fusion protein of any one of paragraphs 1-14 comprising the structure CH1-hinge-CH2-CH3 or CL-hinge-CH2-CH3, with a VLRB domain at the N-terminus and/or C-terminus.

16. The heavy chain fusion protein of any one of paragraphs 1-15, wherein the CL domain is SEQ ID NO:23 or 24 or variant thereof with at least 70% sequence identity thereto.

17. The heavy chain fusion protein of any one of paragraphs 1-16, wherein the CH1, CH2, and/or CH3 comprises the sequence of the CH1, CH2, and/or CH3 of SEQ ID NOS:25, 26, 62, 63, or 64, or a variant thereof with at least 70% sequence identity thereto.

18. The heavy chain fusion protein of any one of paragraphs 1-17, comprising the amino acid sequence of SEQ ID NOS:25, 26, 62, 63, 64, 65, or 66 or a variant thereof with at least 70% sequence identity thereto.

19. The heavy chain fusion protein of any one of paragraphs 1-18 comprising the amino acid sequence of SEQ ID NOS:53, 55, or 56, with or without the signal sequence, or a variant thereof with at least 70% sequence identity thereto, optionally wherein the VLRB antigen binding domain is not mutated relative to SEQ ID NOS:53, 56, or 56.

20. A heavy chain fusion protein comprising the amino acid sequence of one of SEQ ID NOS:53, 55, or 65 with or without the signal sequence.

21. A light chain fusion protein comprising one or two variable lymphocyte receptor B (VLRB) antigen binding domains and a CL or CH1 domain, wherein the VLRB antigen binding domains are the same or different and optionally wherein the VLRB antigen binding domain(s) are at the N-terminus of CL or CH1 domain, the C-terminus of the CL or CH1 domain, or a combination thereof.

22. The light chain fusion protein of paragraph 21 further comprising a variable region of immunoglobulin light chain (VL), ScFv, VHH, L, or R optionally at the N-terminus or C-terminus of the fusion protein and the VLRB antigen binding domain and mono- or multivalent single-chain variable fragment (ScFv), VHH, a polypeptide ligand (L), or a polypeptide receptor (R) are at different ends of the fusion protein.

23. The light chain fusion protein of paragraph 22 comprising a domain structure of Table 2.

24. A light chain fusion protein comprising one or more VLRB antigen binding domains and a domain structure of Table 2.

25. The light chain fusion protein of any one of paragraphs 21-24, wherein the VLRB antigen binding domain(s) binds to a cancer or tumor antigen or an antigen expressed on by immune cells.

26. The light chain fusion protein of any one of paragraphs 22-25, wherein the variable region of immunoglobulin light chain (VL), ScFv, VHH, L, or R bind to a cancer or tumor antigen or an antigen expressed on by immune cells.

27. The light chain fusion protein of any one of paragraphs 1-26, wherein each of the immunoglobin domains are independently selected from a mammalian, optionally human, IgA, IgD, IgE, IgG, and IgM, or a variant thereof with at 70% sequence identity thereto.

28. The light chain fusion protein of paragraph 27, wherein the IgG is IgG1, IgG2, IgG3, and/or IgG4 and/or the IgA is IgA1 and/or IgA2.

29. The light chain fusion protein of any one of paragraphs 21-28 consisting of the one or two VLRB antigen binding domain or one VLRB antigen binding domains and immunoglobulin light chain (VL), ScFv, VHH, L, or R optionally at the N-terminus or C-terminus of the fusion protein and the VLRB antigen binding domain and mono- or multivalent single-chain variable fragment (ScFv), VHH, a polypeptide ligand (L), or a polypeptide receptor (R) fused to the N-terminus and C-terminus of the CL or CH domain.

30. The light chain fusion protein of any one of paragraphs 21-29, wherein the CL domain is SEQ ID NO:23 or 24 or variant thereof with at least 70% sequence identity thereto.

31. The light chain fusion protein of any one of paragraphs 21-29, wherein the CH1 comprises the sequence of the CH1 of SEQ ID NOS:25, 26, 62, 63, 64, 65, or 66 or a variant thereof with at least 70% sequence identity thereto.

32. The light chain fusion protein of any one of paragraphs 21-31, comprising the amino acid sequence of SEQ ID NOS:24, or a variant thereof with at least 70% sequence identity thereto.

33. The light chain fusion protein of any one of paragraphs 1-18 comprising the amino acid sequence of SEQ ID NO:54 with or without the signal sequence, or a variant thereof with at least 70% sequence identity thereto, optionally wherein the VLRB antigen binding domain is not mutated relative to SEQ ID NO:54.

34. A light chain fusion protein comprising the amino acid sequence of SEQ ID NO:54 with or without the signal sequence.

35. The heavy chain of any one of paragraphs 1-20 and/or the heavy chain of any one of paragraphs 21-33 further comprising an active agent cargo conjugated thereto.

36. A nucleic acid encoding the fusion protein of any one of paragraphs 1-34.

37. The nucleic acid of paragraph 36 further comprising an expression control sequence.

38. A cell comprising the nucleic acid of paragraphs 36 or 37.

39. A chimeric antibody comprising two heavy chain fusion proteins according to any one of paragraphs 1-20 and two light chain fusion proteins according to any one of paragraphs 21-34.

40. The antibody of paragraph 39, wherein the two heavy chain fusion proteins are the same.

41. The antibody of paragraph 39 wherein the two heavy chain fusion proteins are different.

42. The antibody of any one of paragraphs 39-41 wherein the two light chain fusion proteins are the same.

43. The antibody of any one of paragraphs 39-42, wherein the two light chain fusion proteins are different.

44. The antibody of any one of paragraphs 39-43, wherein the antibody is monospecific.

45. The antibody of any one of paragraphs 39-43, wherein the antibody is bispecific.

46. The antibody of any one of paragraphs 39-43, wherein the antibody is multispecific.

47. The antibody of any one of paragraphs 39-47, comprising a structure according to Table 3, Table 4, or any of FIG. 2, 3A-3D, 4, 5, or 6.

48. A chimeric antibody comprising SEQ ID NO:53 forming a tetrameric antibody structure with itself, and/or one or more of MM3 VLRB L chains, optionally SEQ ID NO:54.

49. A chimeric antibody comprising at least one VLRB antigen binding domain, wherein the antibody comprises SEQ ID NO:55 forming a tetrameric antibody optionally with MM3 VLRB hole H chain, optionally SEQ ID NO:56, and/or one or more of MM3 VLRB L chains optionally SEQ ID NO:54.

50. A chimeric antibody comprising at least one VLRB antigen binding domain, wherein the antibody comprises SEQ ID NO:65 forming a tetrameric antibody structure optionally with MM3 VLRB hole H chain optionally SEQ ID NO:56, and/or one or more of MM3 VLRB L chain optionally SEQ ID NO:54.

51. A chimeric antibody comprising at least one VLRB antigen binding domain, wherein the antibody comprises SEQ ID NO:66 forming a tetrameric antibody structure optionally with MM3 VLRB hole H chain optionally SEQ ID NO:56, and/or one or more of MM3 VLRB L chain optionally SEQ ID NO:54.

52. A chimeric antibody comprising two heavy chains independently selected from Table 1 and two light chains independently selected from Table 2, wherein the antibody comprises at least one VLRB antigen binding domain.

53. A chimeric antibody comprising a structure according to Table 3, Table 4, or any of FIG. 2, 3A-3D, 4, 5, or 6, wherein the antibody comprises at least one VLRB antigen binding domain.

54. A chimeric antibody comprising a dimer of a VLRB antigen binding domain fused to a hinge-CH2-CH3.

55. The antibody of paragraph 54 comprising a dimer of the amino acid sequence of any one of SEQ ID NOS:51 or 52.

56. The antibody of any one of paragraphs 39-55, wherein the antibody can bind to a cancer or tumor antigen.

57. The antibody of any one of paragraphs 39-56, wherein the antibody can bind to an immune cell.

58. The antibody of any one of paragraphs 39-57, wherein the antibody can bind to both a cancer or tumor antigen and an immune cell.

59. The antibody of paragraphs 57 or 58, wherein the immune cell is a natural killer (NK) cell or a macrophage.

60. The antibody of any one of paragraphs 39-59, wherein the antibody has antibody-dependent cellular cytotoxicity (ADCC), complement-dependent cytotoxicity (CDC), and/or antibody-dependent cellular phagocytosis (ADCP) activity.

61. The antibody of any one of paragraphs 39-62, wherein the antibody can induce T cell activation, T cell proliferation, T cell killing of a target cell, or a combination thereof.

62. The antibody of any one of paragraphs 39-61 comprising an active agent cargo conjugated thereto.

63. A composition comprising the antibody of any one of paragraphs 39-62.

64. The composition of paragraph 63 in an effective amount to induce a therapeutic or diagnostic result in a subject in need thereof.

65. The composition of paragraphs 63 or 64 in a formulation suitable for parenteral or enteral administration.

66. A method of treating a subject in need thereof comprising administering the subject the composition of any one of paragraphs 63-65.

67. A method of inducing an immune response in a subject in need thereof comprising administering the subject the composition of any one of paragraphs 63-65.

68. A method of treating a subject for cancer comprising administering the subject the composition of any one of paragraphs 63-65.

69. The method of paragraph 68, wherein the antibody binds to cells of the cancer.

70. A method of treating a subject for an infection comprising administering the subject the composition of any one of paragraphs 63-65.

71. The method of paragraph 70, wherein the antibody binds to infected cells.

72. The method of any one of paragraphs 66-71, wherein the antibody binds to one or more immune cell types.

73. The method of any one of paragraphs 66-72, comprising an immune response selected from antibody-dependent cellular cytotoxicity (ADCC), complement-dependent cytotoxicity (CDC), and/or antibody-dependent cellular phagocytosis (ADCP) activity.

74. Any of the foregoing paragraphs wherein the antibody includes an anti-CD3 antigen binding domain/antibody specific for T cells, or includes an anti-CD8 antigen binding domain or CD8 ligand TL antigen and is specific for cytotoxic T cells.

75. Any of the foregoing paragraphs wherein the fusion protein(s) and/or antibodies wherein one or more of the VLRB domains is an MM3 VLRB antigen binding domain optionally in combination with an ScFv domain comprising an anti-CD3 or anti-CD8 antigen binding domain, or CD8 ligand optionally wherein the ligand is thymus leukemia antigen.

76. Any of the foregoing paragraphs comprising the anti-CD3 binding domain of SEQ ID NO:22 or SEQ ID NO:34.

77. The antibody of any one of paragraphs 68-70 having the structure of any one of FIG. 3A, 3B, 3C, 3D, 4, 5, or 6.

78. A fusion protein comprising a VLRB antigen binding domain fused to a hinge-CH2-CH3.

79. The fusion protein of paragraph 78 comprising the amino acid sequence of any one of SEQ ID NOS:51 or 52.

### EXAMPLES

### Example 1: Construction of Chimeric VLRB-IgG Antibodies, and Binding Thereof, and Immune Response Modulation Therewith

### Materials and Methods

### Cells and Reagents.

All human hemopoietic cell lines were provided by M. Cooper (Emory University, Atlanta, Georgia, USA) and were cultured in RPMI 1640 supplemented with glutamine, 100 U/ml penicillin-streptomycin, 50 pM 2-mercaptoethanol, and 10% FBS (complete media). Cells were maintained in a humidified atmosphere at 37° C. and 5% C02. De-identified anticoagulated human blood samples and sera were provided by Yerkes Primate Research Center (Emory University). Human peripheral blood mononuclear cells (PBMCs) were isolated by density gradient centrifugation over Lymphoprep™ gradient medium (Stem Cell Technologies, Catalog #07801). Mouse monoclonal Abs against cell-surface antigens CD19, CD38, CD3, BCMA, CD4, CD8, CD25, and CD69; CD38-specific therapeutic antibody Darzalex (daratumummab) and Fluorophore-labeled goat anti-mouse IgG secondary Ab were commercially sourced.

Monoclonal VLRB₂ Human IgG1 Fc, MM3 VLRB₄ Human IgG1 and MM3 VLRB₄:Anti-CD3 scFv Human IgG1 Fusion Proteins.

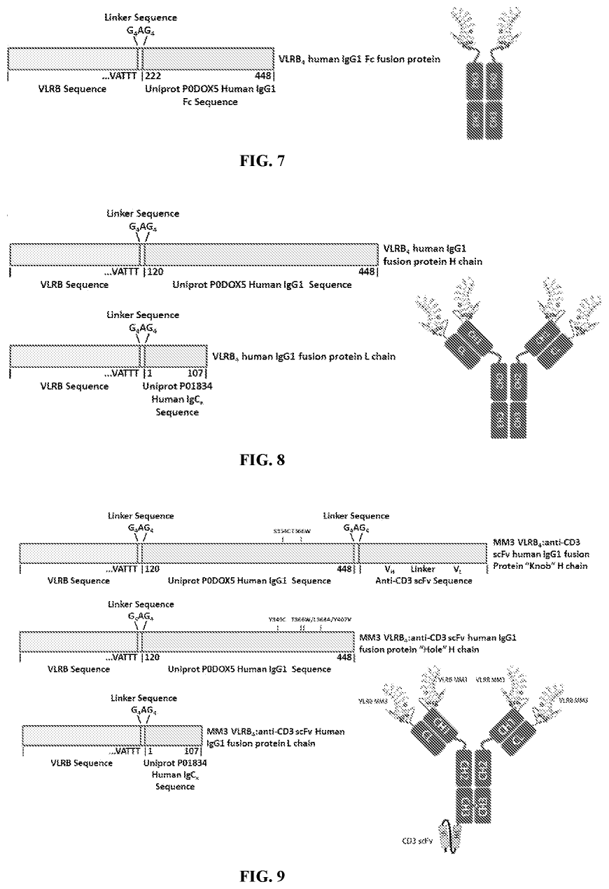

The isolation and binding properties of monoclonal VLRB Abs MM3 (Yu, et al., “Identification of human plasma cells with a lamprey monoclonal antibody.” JCI Insight. 2016; 1(3). Epub 2016/05/07. doi: 10.1172/jci.insight.84738. PubMed PMID: 27152361; PMCID: PMC4854299.), N8 (Chan, et al., “A tyrosine sulfation-dependent HLA-I modification identifies memory B cells and plasma cells.” Sci Adv. 2018; 4(11):eaar7653. Epub 2018/11/13. doi: 10.1126/sciadv.aar7653. PubMed PMID: 30417091; PMCID: PMC6221509.) and O13 (Collins, et al., “Structural Insights into VLR Fine Specificity for Blood Group Carbohydrates.” Structure. 2017; 25(11):1667-78 e4. Epub 2017/10/11. doi: 10.1016/j.str.2017.09.003. PubMed PMID: 28988747; PMCID: PMC5677568.) have been previously described. Bivalent MM3, N8 and O13 VLRB₂ human IgG1 Fc and tetravalent MM3 VLRB₄ human IgG1 fusion proteins (FIGS. 7 and 8) and the MM3 VLRB:anti-CD3 scFv human IgG1 bispecific T cell engager (BiTe) (FIG. 9) were produced by Curia-LakePharma with their TunaCHO™ recombinant protein expression technology and purified from culture media by protein A affinity chromatography. The BiTe design incorporates “knob-in-hole” mutations to facilitate correct H chain pairing (Carter, et al., “Bispecific IgG by Design.” J. Immunol. Meth. 248: 7-15, (2001)) and “LALA PG” mutations to silence FcRI, FcRII and FcRIII binding (Lo, et al., “Effector-Attenuating Substitutions That Maintain Antibody Stability and Reduce Toxicity in Mice.” J. Biol. Chem. 292: 3900-3908, (2017)).

The BiTe structure is tetravalent for MM3 VLRB designed to improve tumor cell binding and monovalent for the anti-CD3 scFv designed to avoid crosslinking CD3 and T cell activation in the absence of MM3 VLRB tumor cell engagement. The anti-CD3 scFv sequence is from WO 2007/073499A2 sequence identifier 65. The MM3 VLRB sequence is from U.S. Pat. No. 10,167,330 B2 sequence identifier 57.

The O13 VLRB antibody recognizes the human 0 blood group type 2 H trisaccharide antigen (H3), Fucose β1,2 Galactose β1,4 N-acetylglucosamine.

Unless otherwise noted, the genes for all constructs were synthesized by LakePharma, and encoded proteins produced by transient expression with their proprietary vector/CHO cell recombinant protein expression technology, and purified from culture media by protein A chromatography.

### (1) VLRB Human IgG1 Fc Fusion Proteins

(a) Bivalent MM3 VLRB2 human IgG1 Fc
Signal Sequence (dashed underlining)
MM3 VLRB Sequence (bold)
Linker Sequence (lowercase)
Human IgG1 Hinge-C_{H}2-C_{H}3 Fc Sequence (italics)
(SEQ ID NO: 51)

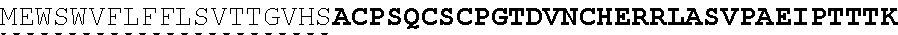

ILRLYINQITKLEPGVFDRLTQLTQLGLWDNQLQALPEGVEDRLVNLQKLY
LNQNQLLALPVGVFDKLTQLTYLDLNNNQLKSIPRGAFDNLKSLTHIWLYG
NPWDCECSDILYLKNWIVQHASIVNPHPYGGVDNVKCSGTNTPVRAVTEAS
TSPSKCPGYVATTTggggaggggDKTHTCPPCPAPELLGGPSVFLFPPKPK
DTLMISRTPEVTCVVVDVSHEDPEVKFNWYVDGVEVHNAKTKPREEQYNST
YRVVSVLIVLHQDWLNGKEYKCKVSNKALPAPIEKTISKAKGQPREPQVYT
LPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPG*
*C-terminal Lysine removed per Cai, et al. (2011)
Biotechnol Bioeng 108: 404-412 doi: 10.1002/bit.22933.
Other VLRB human IgG1 Fc fusion proteins were produced by
replacing the MM3 VLRB sequence with the sequence for the
new VLRB and maintaining all other sequences of the above
construct as shown belowfor the O13 VLRB2 human IgG1 Fc
fusion protein.
(b) Bivalent O13 VLRB2 human IgG1 Fc

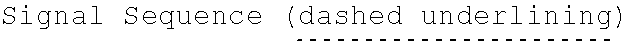

O13 VLRB Sequence (bold)
Linker Sequence (lowercase)
Human IgG1 Hinge-C_{H}2-C_{H}3 Fc Sequence (italics)
(SEQ ID NO: 52)

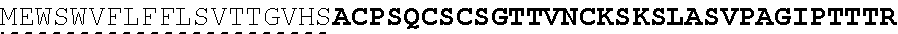

VLYLNDNQITKLEPGVFDRLVNLQTLWLNNNQLTSLPAGLFDSLTQLTILA
LDSNQLQALPVGVFGRLVDLQQLYLGSNQLSALPSAVEDRLVHLKELLMCC
NKLTELPRGIERLTHLTHLALDQNQLKSIPHGAFDRLSSLTHAYLFGNPWD
CECRDIMYLRNWVADHTSIVMRWDGKAVNDPDSAKCAGTNTPVRAVTEAST
SPSKCPGYVATTTggggaggggDKTHTCPPCPAPELLGGPSVFLFPPKPKD
TLMISRTPEVTCVVVDVSHEDPEVKFNWYVDGVEVHNAKTKPREEQYNSTY
RVVSVLIVLHQDWLNGKEYKCKVSNKALPAPIEKTISKAKGQPREPQVYTL
PPSREEMTKNQVSLTCLVKGFYPSDIAVEWESNGQPENNYKTTPPVLDSDG
SFFLYSKLTVDKSRWQQGNVFSCSVMHEALHNHYTQKSLSLSPG*
*C-terminal Lysine removed per Cai, et al. (2011)
Biotechnol Bioeng 108: 404-412 doi: 10.1002/bit.22933.

### (2) Tetravalent MM3 VLRB4 Human IgG1

(a) MM3 VLRB human IgG1 H Chain construct

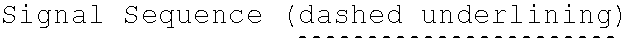

MM3 VLRB Sequence (bold)
Linker Sequence (lowercase)
Human IgG1 C_{H}1-Hinge-C_{H}2-C_{H}3 Sequence (italics)
(SEQ ID NO: 53)

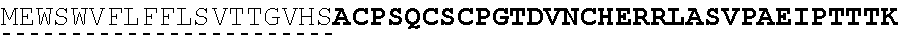

ILRLYINQITKLEPGVFDRLTQLTQLGLWDNQLQALPEGVEDRLVNLQKLY
LNQNQLLALPVGVFDKLTQLTYLDLNNNQLKSIPRGAFDNLKSLTHIWLYG
NPWDCECSDILYLKNWIVQHASIVNPHPYGGVDNVKCSGTNTPVRAVTEAS
TSPSKCPGYVATTTggggaggggASTKGPSVFPLAPSSKSTSGGTAALGCL
VKDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYNS
TYRVVSVLIVLHQDWLNGKEYKCKVSNKALPAPIEKTISKAKGQPREPQVY
TLPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPG*
(b) MM3 VLRB human IgKappa L Chain construct

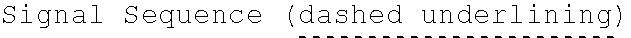

MM3 VLRB Sequence (bold)
Linker Sequence (lowercase)
Human IgKappa C_{L} Sequence (italics)
(SEQ ID NO: 54)

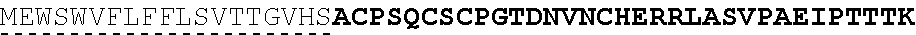

ILRLYINQITKLEPGVFDRLTQLTQLGLWDNQLQALPEGVFDRLVNLQKLY
LNQNQLLALPVGVFDKLTQLTYLDLNNNQLKSIPRGAFDNLKSLTHIWLYG
NPWDCECSDILYLKNWIVQHASIVNPHPYGGVDNVKCSGTNTPVRAVTEAS
TSPSKCPGYVATTTggggaggggRTVAAPSVFIFPPSDEQLKSGTASVVCL
LNNFYPREAKVQWKVDNALQSGNSQESVTEQDSKDSTYSLSSTLTLSKADY
EKHKVYACEVTHQGLSSPVTKSENRGEC

(3) MM3 VLRB4:TR66 Anti-CD3 scFv Human IgG1 BiTe

Utilizes “Knob-in-Hole” technology to facilitate correct H chain pairing and LALA PG mutations to silence FcR1, II and III binding while retaining FcRn binding to impart the dersiable blood T_{1/2} of IgG1 antibody.

All constructs are designed to increase/enhance tumor binding by incorporating multivalent MM3 VLRB presentation and to avoid crosslinking CD3 which would lead to T cell activation in the absence of tumor engagement, i.e., nonspecific T cell activation and killing, by incorporating monovalent anti-CD3 scFv presentation.

(a) MM3 VLRB human IgG1 H Chain S354C/T366W
“Knob” with C-terminal TR66 anti-CD3 scFv construct with
L234A/L235A/P329G FcR1, II, III silencing changes

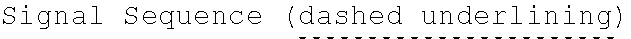

MM3 VLRB Sequence (bold)
Linker Sequence (lowercase)
Human IgG1 C_{H}1-Hinge-C_{H}2-C_{H}3 Sequence (italics with
L344A/L345A/P439G (also referred to elsewhere as
“L234A/L235A/P329G”) “LALA PG” FcR1, RII, RIII silencing;
S464C/T476W (also referred elsewhere was “S354C/T366W”)
“Knob” mutations illustrated with italics/bold/single
underlining)
TR66 anti-CD3 scFv Sequence (double underlining)
(SEQ ID NO: 55)

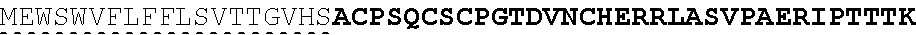

ILRLYINQITKLEPGVFDRLTQLTQLGLWDNQLQALPEGVEDRLVNLQKLY
LNQNQLLALPVGVFDKLTQLTYLDLNNNQLKSIPRGAFDNLKSLTHIWLYG
NPWDCECSDILYLKNWIVQHASIVNPHPYGGVDNVKCSGTNTPVRAVTEAS
TSPSKCPGYVATTTggggaggggASTKGPSVFPLAPSSKSTSGGTAALGCL
VKDYFPEPVTVSWNSGALTSGVHTFPAVLQSSGLYSLSSVVTVPSSSLGTQ
TYICNVNHKPSNTKVDKKVEPKSCDKTHTCPPCPAPEAAGGPSVFLFPPKP
KDTLMISRTPEVTCVVVDVSHEDPEVKENWYVDGVEVHNAKTKPREEQYNS
TYRVVSVLIVLHQDWLNGKEYKCKVSNKALGAPIEKTISKAKGQPREPQVY
TLPPCREEMTKNQVSLWCLVKGFYPSDIAVEWESNGQPENNYKTTPPVLDS
DGSFFLYSKLTVDKSRWQQGNVFSCSVMHEALHNHYTQKSLSLSPGgggga
ggggDVQLVQSGAEVKKPGASVKVSCKASGYTFTRYTMHWVRQAPGQGLEW
IGYINPSRGYTNYADSVKGRFTITTDKSTSTAYMELSSLRSEDTATYYCAR
YYDDHYCLDYWGQGTTVTVSSGEGTSTGSGGSGGSGGADDIVLTQSPATLS
LSPGERATLSCRASQSVSYMNWYQQKPGKAPKRWIYDTSKVASGVPARESG
SGSGTDYSLTINSLEAEDAATYYCQQWSSNPLTFGGGTKVEIKS
(b) MM3 VLRB human IgG1 H Chain
Y349C/T366S/L368A/Y407V “Hole” construct with
L234A/L235A/P329G FcR1, II, III silencing changes

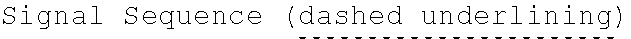

MM3 VLRB Sequence (bold)
Linker Sequence (lowercase)
Human IgG1 C_{H}1-Hinge-C_{H}2-C_{H}3 Sequence (italics with
L344A/L345A/P439G (also referred to elsewhere as
“L234A/L235A/P329G”) “LALA PG” FcR1, RII, RIII silencing;
Y459C/T476S/L478A/Y517V (also referred to elsewhere as
“Y349C/T366S/L368A/Y407V”) “Hole” mutations illustrated with
italics/bold/single underlining)
(SEQ ID NO: 56)

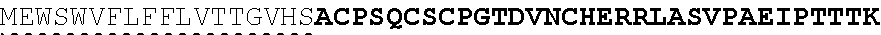

ILRLYINQITKLEPGVFDRLTQLTQLGLWDNQLQALPEGVEDRLVNLQKLY
LNQNQLLALPVGVFDKLTQLTYLDLNNNQLKSIPRGAFDNLKSLTHIWLYG
NPWDCECSDILYLKNWIVQHASIVNPHPYGGVDNVKCSGTNTPVRAVTEAS
TSPSKCPGYVATTTggggaggggASTKGPSVFPLAPSSKSTSGGTAALGCL
VKDYFPEPVTVSWNSGALTSGVHTFPAVLQSSGLYSLSSVVTVPSSSLGTQ
TYICNVNHKPSNTKVDKKVEPKSCDKTHTCPPCPAPEAAGGPSVFLFPPKP
KDTLMISRTPEVTCVVVDVSHEDPEVKENWYVDGVEVHNAKTKPREEQYNS
TYRVVSVLTVLHQDWLNGKEYKCKVSNKALGAPIEKTISKAKGQPREPQVC
TLPPSREEMTKNQVSLSCAVKGFYPSDIAVEWESNGQPENNYKITPPVLDS
DGSFFLVSKLTVDKSRWQQGNVFSCSVMHEALHNHYTQKSLSLSPG*

Purity of recombinant fusion proteins was assessed by SE-UPLC chromatography and molecular weights determined by reducing and nonreducing SDS gel electrophoresis. Yields, purity, molecular weights and extinction coefficients for the recombinant fusion proteins are presented in Table 10.

TABLE 10
Properties of recombinant VLRB IgG1 fusion proteins.

| VLRB human Ig fusion proteins | Yield (mg/L)¹ | Purity | Ext. Cof. | Mol. wt. (daltons) |
|---|---|---|---|---|
| MM3 VLRB₂ human IgG1 Fc | 260 | 98.75% | 1.41 | 121,570 |
| O13 VLRB₂ human IgG1 Fc | 179 | 95.13% | 1.219 | 123,900 |
| N8 VLRB₂ human IgG1 Fc | 360 | 92.86% | 1.213 | 127,070 |
| MM3 VLRB₄ human IgG1 | 148 | 97.76% | 1.307 | 184,000 |
| VLRB₄:anti-CD3 scFv human IgG1 BiTe² | 247 | 78.00% | 1.47 | 219,620 |

¹Post-protein A purification; 30 mL shake flask cultures of transient trasfectants.
²Purity post-SEC chromatography was 99.10% with a final yield of 108 mg/L.

### Binding Assays.

Binding of VLRB fusion proteins and other Ab reagents to cells was assayed by flow cytometry with a Miltenyi MACSQuant™ Analyzer 10 Flow Cytometer. Bivalent VLRB₂ human IgG1 Fc and tetravalent VLRB₄ human IgG1 fusion proteins and daratumumab were directly labeled with fluorophore using the Zenon™ human IgG labeling kit (Thermofisher, Catalog #Z25402). Cells were incubated on ice for 20 minutes with blocking buffer (Southern Biotech, Catalog #0060-01), strained with snap cap cell strainer (Vendor, Catalog #60819-524), and transferred to the wells of a 96 well microtiter plate at 400,000 cells per well and isolated by centrifugation at 300×g for 5 minutes at 4° C. Cells were resuspended in 100 μL PBS—2% FBS containing the indicated concentrations of fluorophore-labeled VLRB fusion proteins and incubated for 30 minutes on ice in PBS—2% FBS and washed twice with cold PBS—2% FBS prior to flow cytometry analysis. For other Ab reagents, cells in the microtiter plate wells were resuspended with 100 μL of PBS—2% FBS containing the indicated concentrations of unlabeled Ab reagent for 30 minutes on ice in PBS—2% FBS, followed by detection of bound Ab reagent with fluorophore-labeled goat anti-mouse Ab. Dead cells were excluded by inclusion of propidium iodide (1 μg/ml). Flow cytometric data were analyzed using the FlowJo software package.

### Complement Dependent Cytotoxicity (CDC) Assays.

Activation of complement dependent cytotoxicity (CDC) by VLRB fusion proteins and other Abs was assessed by flow cytometry with PI staining to quantitate dead cells. VLRB fusion proteins and other Abs at the indicated concentrations were incubated with the indicated numbers of target cells for 30 minutes on ice in PBS—2% FBS, washed once with cold PBS—2% FBS, resuspended in RPMI 1640 containing 0%, 20% or 40% human serum as a source of complement and incubated for 4 hours in a humidified atmosphere at 37° C. and 5% CO₂ prior to staining with PI and flow cytometry analysis.

### Antibody-Dependent Cellular Cytotoxicity (ADCC) Assays.

Activation of antibody-dependent cellular cytoxicity (ADCC) by VLRB fusion proteins and other Abs was assessed with human PBMC effector cells by detecting lactate dehydrogenase (LDH) released into cell culture media with the Promega ADCC Reporter Bioassay kit (Promega catalog #G7015). VLRB fusion proteins or other Abs at the indicated concentrations were incubated for 30 minutes on ice with the indicated numbers of target cells in PBS—2% FBS, washed with cold PBS—2% FBS, resuspended in complete media containing the indicated numbers of Jurkat cells stably expressing human FcγRIIIa V158 (high affinity) linked to NFAT-induced luciferase and incubated for 6 hours in a humidified atmosphere at 37° C. and 5% CO². Luminescience was measured with a FLUOstar® Omega multi-mode microplate reader (BMG Labtech).

### Activation of PBMC Assays.

Activation of human PBMC T cells by MM3 VLRB BiTe protein was assessed with Jurkat TCR/CD3 NFAT cells (Promega catalog #J1621) and by flow cytometry measurement of PBMC T cell CD25 and CD69 cell surface expression. Assays with the Jurkat TCR/CD3 NFAT cells followed kit instructions. For assays with human PBMCs the indicated concentrations of MM3 VLRB BiTe protein were incubated with the indicated number of target cells on ice for 30 minutes in PBS—2% FBS, washed once with cold PBS—2% FBS and then resuspended in complete media containing the indicated number of PBMCs and incubated overnight in a humidified atmosphere at 37° C. and 5% CO₂ prior to staining with anti-CD25 and anti-CD4, anti-CD25 and anti-CD8, anti-CD69 and anti-CD4, and anti-CD69 and anti-CD8 Ab combinations to visualize activation of both CD4 and CD8 PBMC T cells as measured with both CD25 and CD69 cell surface proteins.

### Activation of Cytotoxic T Cell Killing of Target Cells.

Activation of cytotoxic T cell killing of target cells by the MM3 VLRB BiTe was assessed with human PBMCs as a source of T cells and Daudi target cells by detecting lactate dehydrogenase (LDH) released into cell culture media with the Promega ADCC Reporter Bioassay kit (Promega catalog #G7015). MM3 VLRB BiTe protein at the indicated concentrations was incubated for 30 minutes on ice with the indicated numbers of target cells in PBS—2% FBS, washed with cold PBS—2% FBS, then resuspended in complete media containing the indicated numbers of human PBMCs, and incubated for 4 hours in a humidified atmosphere at 37° C. and 5% CO₂. Luminescience was measured with a FLUOstar® Omega multi-mode microplate reader (BMG Labtech).

### Results

### Binding Properties of VLRB Ig Fusion Proteins.

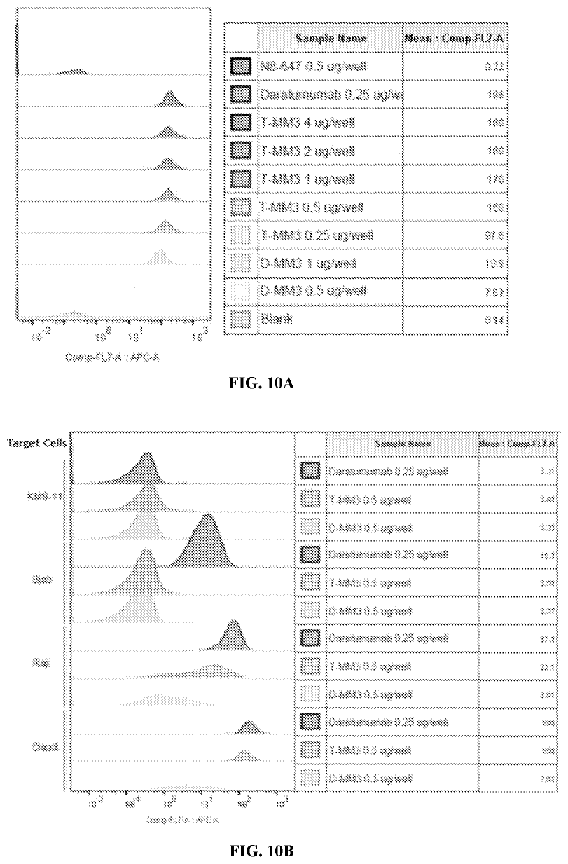

The binding properties of MM3, N8 and O13 VLRB₂ human IgG1 Fc, MM3 VLRB₄ human IgG1 and MM3 VLRB₄:anti-CD3 scFv human IgG1 bispecific T cell engager (BiTe) fusion proteins were assessed by flow cytometry with results summarized in Table 11 and example flow cytometry data shown in FIGS. 10-13. The specificity of the N8 VLRB for sulfated-Tyrosine modified HLA class 1 antigens (Chan, et al. “A tyrosine sulfation-dependent HLA-I modification identifies memory B cells and plasma cells.” Sci Adv. 2018; 4(11):eaar7653. Epub 2018/11/13. doi: 10.1126/sciadv.aar7653. PubMed PMID: 30417091; PMCID: PMC6221509.), the O13 VLRB for the 0 blood group type 2 H trisaccharide antigen (Collins, et al., “Structural Insights into VLR Fine Specificity for Blood Group Carbohydrates.” Structure. 2017; 25(11):1667-78 e4. Epub 2017/10/11. doi: 10.1016/j.str.2017.09.003. PubMed PMID: 28988747; PMCID: PMC5677568.) and the MM3 VLRB for higher order structures, e.g., tetramers, of CD38 (Yu, et al., “Identification of human plasma cells with a lamprey monoclonal antibody.” JCI Insight. 2016; 1(3). Epub 2016/05/07. doi: 10.1172/jci.insight.84738. PubMed PMID: 27152361; PMCID: PMC4854299.) have been described. The cell line binding specificities of these VLRB fusion proteins shown in Table 11 and FIGS. 10-13 are in agreement with the data reported for the “parental” VLRB antibodies.

All cell lines that are known to express CD38 are positive for binding by daratumumab and by the anti-CD38 HIT mAb. Cell lines that express high amounts of CD38, Daudi and Raji, are also positive for binding by MM3 VLRB₂ human IgG1 Fc and MM3 VLRB₄ human IgG1, but cell lines such as BJAB that express low levels of CD38 are negative for MM3 VLRB binding. Binding of the tetravalent MM3 VLRB₄ human IgG1 is superior to that of the bivalent MM3 VLRB₂ human IgG1 Fc. Specificity of binding is supported by the absence of binding to KMS-11 CD38⁻ cells by all of the anti-CD38 reagents, by absence of binding to Daudi, Raji and BJAB N8⁻ cells for the N8 reagent, and by N8 VLRB₂ human IgG1 Fc binding, though weak, to the N8⁺ KMS-11 cells; Lastly, it is likely that MM3 recognizes a “neotope” that is created when CD38 forms higher order structures, i.e., tetramers, and that high CD38 expression is needed to drive via mass action formation of MM3 VLRB detectable CD38 tetramers.

### Activation of CDC and ADCC Immune Effector Functions.

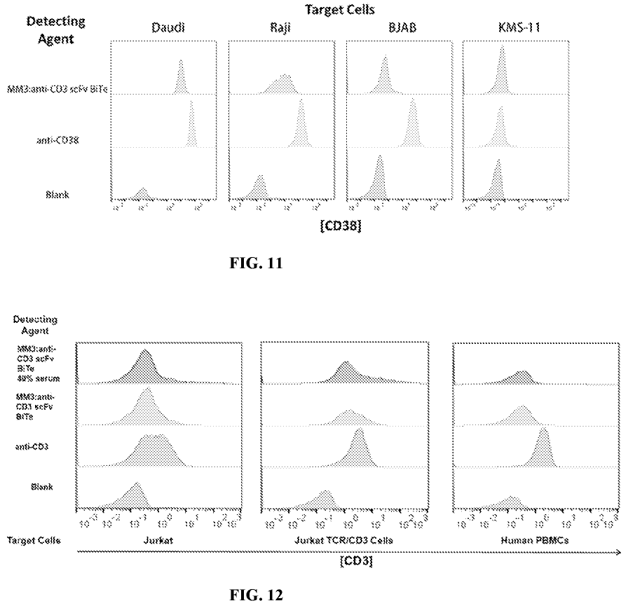

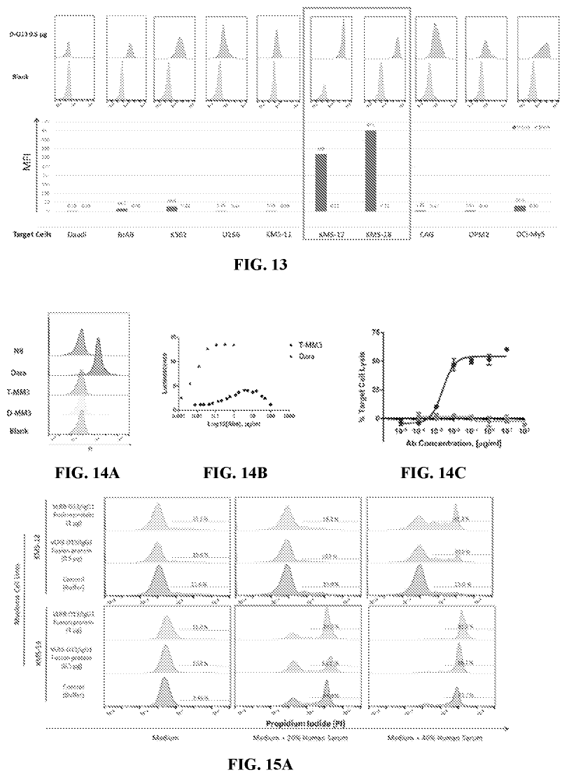

FIGS. 14A-14C report results of assays to measure activation of CDC and ADCC immune effector functions by VLRB fusion proteins. At the protein (0.5 μg) and complement (20% human serum) amounts tested neither the bivalent MM3 VLRB₂ human IgG1 Fc nor the tetravalent MM3 VLRB₄ human IgG1 fusion protein demonstrated detectable CDC activation activity (FIG. 14A). The tetravalent MM3 VLRB human IgG1 fusion protein (FIG. 14B) but not the bivalent MM3 VLRB human IgG1 Fc fusion protein (FIG. 14C) demonstrated activation of FcrRIII (CD16a)-mediated ADCC effector function, though with substantially poorer potency than the positive control daratumumab IgG1 antibody. The increased valency and avidity-enhanced apparent affinity of the MM3 VLRB₄ IgG1 structure versus the MM3 VLRB₂ IgG1 Fc structure appears to result in a higher density of IgG1 Fc binding sites for stable binding and crosslinking of CD16a, though substantially less than achieved with the positive control anti-CD38 IgG1 mAb daratumumab.

The absence of or poor effector activity of MM3 VLRB fusion proteins is not an intrinsic property of VLRB IgG fusion proteins as illustrated by the potent CDC and ADCC activation property of the O13 VLRB₂ human IgG1 Fc fusion protein (FIG. 15A-15B). The O13 VLRB target, the blood group O type 2 H trisaccharide glycan antigen, is present at sufficient density on the surface of KMS-12 and KMS-18 cells to “aggregate” O13 VLRB₂ IgG1 Fc binding sites for: (a) stable binding of complement C1q component to initiate the antibody-mediated “classical” complement pathway for cell lysis and (b) stable binding and crosslinking of CD16a Fc receptors (FcγRIIIa) to activate lysis of target cells; whereas, the cell surface density of the MM3 VLRB target, CD38 tetramers, of Daudi (and other) cells is not adequate to aggregate MM3 VLRB₂ IgG1 Fc C1q or FcrRIII binding sites for stable binding or crosslinking. Note that though present on BJAB cells the H trisaccharide is substantially less abundant on BJAB cells than on KMS-12 and 18 cells (FIG. 13) and its density is not sufficient to fully activate ADCC with BJAB target cells (FIG. 15B).

MM3 VLRB₄:Anti-CD3 scFv Human IgG1 T Cell Activation.

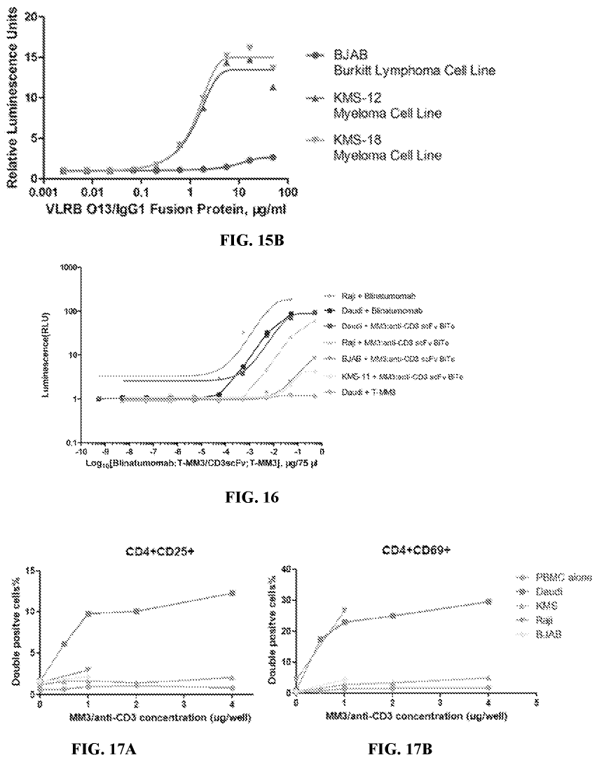

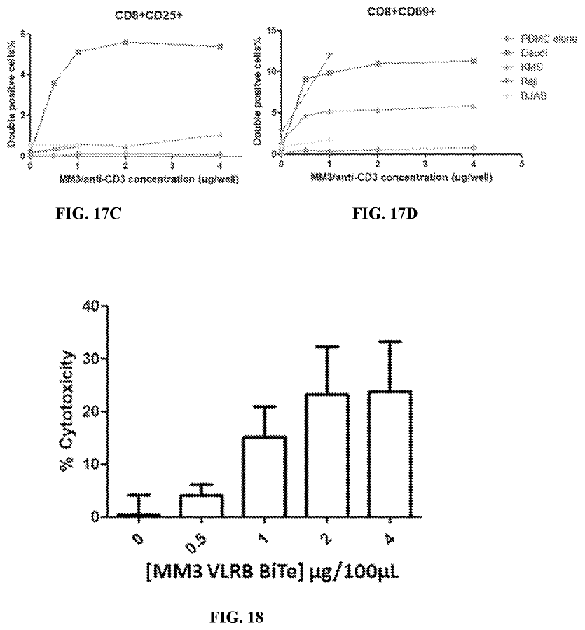

Activation of T cells by the MM3 VLRB BiTe was assessed with Jurkat TCR/CD3 NFAT effector cells (Promega catalog #J1621) (FIG. 16) and with human PBMC T cells by flow cytometry measurement of PBMC T cell CD25 and CD69 cell surface expression (FIG. 17A-17D). Target cells utilized in the assays are as indicated in the figures. Activation of Jurkat TCR/CD3 NFAT cells cells with Daudi target cells by the MM3 VLRB BiTe is comparable to activation by the positive control blinatumomab anti-CD19 scFv:anti-CD3 BiTe, indicating that CD19 expression is comparable to expression of the MM3 CD38 tetramer target by Daudi cells. Note that the molecular weight of the MM3 VLRB BiTe is over 4-fold greater that that of blinatumomab and equal weight amounts of the two BiTes correspond to over 4-fold less molar amount of MM3 VLRB BiTe than blinatumomab. Activation potency of the MM3 VLRB BiTe with the various target cell lines follows the binding intensity of the tetrameric MM3 VLRB human IgG1 fusion protein for these cell lines (see Table 11) indicating that T cell activation activity of the MM3 VLRB BiTe is determined by molecular target expression density. Human PBMC CD8 and CD4 T cells are activated to express cell surface CD25 by the MM3 VLRB₄:anti-CD3 scFv human IgG1 BiTe only when MM3^{hi} target/activator Daudi cells are present, and minimal activation, if any, as measured by CD25 cell surface expression, is detected with other target/activator cells, including MM3^{lo} Raji cells, i.e., T cell activation by MM3 VLRB₄:anti-CD3 scFv human IgG1 as measured with CD25 cell surface expression is antigen specific (FIGS. 17A-17D). Human PBMC CD8 and CD4 T cells are activated to express cell surface CD69 by the MM3 VLRB₄:anti-CD3 scFv human IgG1 BiTe when MM3^{hi} target Daudi cells and MM3^{lo} target Raji cells are present, but not when MM3⁻ BJAB target cells are present. Activation of human PBMC CD8 T cells as measured by CD69 cell surface expression and to a lesser extent by CD25 cell surface expression is seen with the MM3 VLRB₄:anti-CD3 scFv human IgG1 BiTe in the presence of KMS-11 MM3⁻ target cells indicating that KMS-11 cells may be recognized by and able to activate “normal” human PBMC CD8 T cells. Cell surface expression of CD69 appears to be a more sensitive OR less specific indicator of T cell activation than cell surface expression of CD25.

MM3 VLRB₄:Anti-CD3 scFv Human IgG1 Activation of T Cell Killing.

Results for activation of human PBMC T cells to lyse Daudi target cells are shown in FIG. 18. As indicated, the MM3 VLRB BiTe dose-dependently “arms” PBMC T cells to kill Daudi cells.

TABLE 11
Summary of flow cytometry data for VLRB fusion proteins and control antibodies.

| | MFI Values All [ ]s | | | | | |
|---|---|---|---|---|---|---|
| | Cell Lines | | | | | |
| Reagents | KMS-11 | BJAB | Raji | Daudi | KMS-12 | KMS-18 |
| Anti-CD38 HIT2 mAb | <1 | 42-72 | 117-172 | 200-596 | 62.4 | 44.5 |
| Daratumumab | <1 | 15 | 67 | 196-226 | NT | NT |
| MM3 VLRB₄ human IgG1 | <1 | <1 | 22-34 | 98-180 | 4.12 | 4.31 |
| MM3 VLRB₄: TR66 anti-CD3 scFv | <1 | <1 | 11.7 | 53-332 | 4.37 | 2.58 |
| MM3 VLRB₂ human IgG1 Fc | <1 | <1 | 2.6-4.4 | 3.5-19 | NT | NT |
| N8 VLRB₂ human IgG1 Fc | 10.8 | <1 | <1 | <1 | 1.02 | 4.31 |
| O13 human IgG1 Fc | 14.5 | 14.5 | <1 | <1 | 321 | 538 |

NT = not tested.

### Summary

1) VLRB₂ IgG Fc, VLRB₄ IgG and VLRB₄:anti-CD3 scFv IgG fusion proteins can be produced at acceptable yields with conventional recombinant protein production methods and purified to acceptable purity with protein A affinity chromatography (Table 10).
2) VLRB IgG fusion proteins retain the binding properties of the parent VLRB antibody (Table 11 and FIGS. 10A-13).
3) VLRB₄ IgG fusion proteins demonstrate superior binding versus VLRB₂ IgG Fc fusion proteins (Table 11 and FIGS. 10A-10C).
4) VLRB₄ IgG fusion proteins demonstrate superior activation of immune effector functions, e.g., CDC and ADCC, versus VLRB₂ IgG Fc fusion proteins (FIGS. 14A-14C).
5) VLRB4:anti-CD3 scFv IgG BiTes bind both the molecular target recognized by the VLRB antibody and CD3⁺ T cells (FIGS. 11 and 12).
6) The binding of VLRB₄:anti-CD3 scFv IgG BiTes to cells that express the target recognized by the VLRB antibody and to CD3⁺ T cells activates T cells to lyse cells recognized by the VLRB antibody.
7) The binding of VLRB₄:anti-CD3 scFv IgG BiTes to CD3⁺ T cells alone does NOT activate T cells to lyse other cells NOT recognized by the VLRB antibody, i.e., T cell activation by VLRB BiTes is antigen-specific for the antigen recognized by the VLRB antibody.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A heavy chain fusion protein comprising one or more variable lymphocyte receptor B (VLRB) antigen binding domains and a CH1 immunoglobulin domain (CH1) or a CL immunoglobulin domain (CL), optionally wherein the VLRB is at the N-terminus of fusion protein, the C-terminus of the fusion protein, or a combination thereof.

2. The heavy chain fusion protein of claim 1 further comprising one or more of an immunoglobulin hinge domain (hinge), a CH2 immunoglobulin domain (CH2), a CH3 immunoglobulin domain (CH3), and a CH4 immunoglobulin domain (CH4).

3.-6. (canceled)

7. The heavy chain fusion protein of claim 2 further comprising a variable region of immunoglobulin heavy chain (VH), optionally wherein the VH is at the N-terminus of the fusion protein and the VLRB antigen binding domain and VH domain are fused to different ends of the fusion protein.

8. The heavy chain fusion protein of claim 2 further comprising one or more of a mono- or multivalent single-chain variable fragment (ScFv), VHH, a polypeptide ligand (L), or a polypeptide receptor (R), optionally at the N-terminus or C-terminus of the fusion protein and the VLRB antigen binding domain and mono- or multivalent single-chain variable fragment (ScFv), VHH, a polypeptide ligand (L), or a polypeptide receptor (R) are at different ends of the fusion protein.

9.-10. (canceled)

11. The heavy chain fusion protein of claim 8, wherein one or both of the VLRB antigen binding domains bind to a cancer or tumor antigen or an antigen expressed by immune cells and/or wherein one or more of the VH, ScFv, VHH, L, or R bind to a cancer or tumor antigen or an antigen expressed on by immune cells.

12.-18. (canceled)

19. The heavy chain fusion protein of claim 1 comprising the amino acid sequence of SEQ ID NOS:53, 55, or 56, with or without the signal sequence, or a variant thereof with at least 70% sequence identity thereto, optionally wherein the VLRB antigen binding domain is not mutated relative to SEQ ID NOS:53, 56, or 56.

20. (canceled)

21. A light chain fusion protein comprising one or two variable lymphocyte receptor B (VLRB) antigen binding domains and a CL or CH1 domain, wherein the VLRB antigen binding domains are the same or different and optionally wherein the VLRB antigen binding domain(s) are at the N-terminus of CL or CH1 domain, the C-terminus of the CL or CH1 domain, or a combination thereof.

22. The light chain fusion protein of claim 21 further comprising a variable region of immunoglobulin light chain (VL), ScFv, VHH, L, or R optionally at the N-terminus or C-terminus of the fusion protein and the VLRB antigen binding domain and mono- or multivalent single-chain variable fragment (ScFv), VHH, a polypeptide ligand (L), or a polypeptide receptor (R) are at different ends of the fusion protein.

23.-24. (canceled)

25. The light chain fusion protein of claim 22, wherein the VLRB antigen binding domain(s) binds to a cancer or tumor antigen or an antigen expressed on by immune cells.

26. The light chain fusion protein of claim 22, wherein the variable region of immunoglobulin light chain (VL), ScFv, VHH, L, or R bind to a cancer or tumor antigen or an antigen expressed on by immune cells.

27.-28. (canceled)

29. The light chain fusion protein of claim 22 consisting of the one or two VLRB antigen binding domain or one VLRB antigen binding domains and immunoglobulin light chain (VL), ScFv, VHH, L, or R optionally at the N-terminus or C-terminus of the fusion protein and the VLRB antigen binding domain and mono- or multivalent single-chain variable fragment (ScFv), VHH, a polypeptide ligand (L), or a polypeptide receptor (R) fused to the N-terminus and C-terminus of the CL or CH domain.

30.-32. (canceled)

33. The light chain fusion protein of claim 22 comprising the amino acid sequence of SEQ ID NO:54 with or without the signal sequence, or a variant thereof with at least 70% sequence identity thereto, optionally wherein the VLRB antigen binding domain is not mutated relative to SEQ ID NO:54.

34.-38. (canceled)

39. A chimeric antibody comprising two heavy chain fusion proteins each comprising one or more variable lymphocyte receptor B (VLRB) antigen binding domains and a CH1 immunoglobulin domain (CH1) or a CL immunoglobulin domain (CL), optionally wherein the VLRB is at the N-terminus of fusion protein, the C-terminus of the fusion protein, or a combination thereof and two light chain fusion proteins comprising one or two variable lymphocyte receptor B (VLRB) antigen binding domains and a CL or CH1 domain, wherein the VLRB antigen binding domains are the same or different and optionally wherein the VLRB antigen binding domain(s) are at the N-terminus of CL or CH1 domain, the C-terminus of the CL or CH1 domain, or a combination thereof.

40. The antibody of claim 39, wherein the two heavy chain fusion proteins are the same or different and the two light chain fusion proteins are the same or different.

41.-43. (canceled)

44. The antibody of claim 39, wherein the antibody is monospecific, bispecific, or multispecific.

45.-47. (canceled)

48. The chimeric antibody of claim 39 comprising
(i) SEQ ID NO:53 forming a tetrameric antibody structure with itself, and/or one or more of MM3 VLRB L chains, optionally SEQ ID NO:54;
(ii) at least one VLRB antigen binding domain, wherein the antibody comprises SEQ ID NO:55 forming a tetrameric antibody optionally with MM3 VLRB hole H chain, optionally SEQ ID NO:56, and/or one or more of MM3 VLRB L chains optionally SEQ ID NO:54;
(iii) at least one VLRB antigen binding domain, wherein the antibody comprises SEQ ID NO:65 forming a tetrameric antibody structure optionally with MM3 VLRB hole H chain optionally SEQ ID NO:56, and/or one or more of MM3 VLRB L chain optionally SEQ ID NO:54; or
(iv) at least one VLRB antigen binding domain, wherein the antibody comprises SEQ ID NO:66 forming a tetrameric antibody structure optionally with MM3 VLRB hole H chain optionally SEQ ID NO:56, and/or one or more of MM3 VLRB L chain optionally SEQ ID NO:54.

49.-59. (canceled)

60. The antibody of claim 39, wherein the antibody has antibody-dependent cellular cytotoxicity (ADCC), complement-dependent cytotoxicity (CDC), and/or antibody-dependent cellular phagocytosis (ADCP) activity.

61. (canceled)

62. The antibody of claim 39 comprising an active agent cargo conjugated thereto.

63.-65. (canceled)

66. A method of treating a subject in need thereof comprising administering the subject the composition comprising the antibody of claim 39.

67.-73. (canceled)

74. The antibody of claim 39 wherein the antibody comprises
(i) an anti-CD3 antigen binding domain/antibody specific for T cells, or includes an anti-CD8 antigen binding domain or CD8 ligand TL antigen and is specific for cytotoxic T cells
and/or
(ii) one or more of the VLRB domains is an MM3 VLRB antigen binding domain optionally in combination with an ScFv domain comprising an anti-CD3 or anti-CD8 antigen binding domain, or CD8 ligand optionally wherein the ligand is thymus leukemia antigen.

75.-79. (canceled)
